(19) United States
(12) Patent Application Publication
(10) Pub. No.: US 2024/0377734 A1
(43) Pub. Date: Nov. 14, 2024

(54) RESIST COMPOSITION, RESIST PATTERN FORMING METHOD, COMPOUND, AND ACID DIFFUSION CONTROL AGENT

(71) Applicant: TOKYO OHKA KOGYO CO., LTD., Kawasaki-shi (JP)

(72) Inventor: Takuya Uehara, Kawasaki-shi (JP)

(21) Appl. No.: 18/691,411

(22) PCT Filed: Sep. 21, 2022
(86) PCT No.: PCT/JP2022/035124

§ 371 Date: Mar. 12, 2024

(30) Foreign Application Priority Data
Sep. 24, 2021 (JP) 2021-155753

(51) Int. Cl. G03F 7/004; G03F 7/038; G03F 7/039

(52) U.S. Cl. CPC G03F 7/0045; G03F 7/0382; G03F 7/0397

(57) ABSTRACT

A resist composition containing a base material component (A) and a compound represented by General Formula (d0), in which Rd⁰ represents a condensed cyclic group in which an aromatic ring and an alicyclic ring are condensed, the alicyclic ring in the condensed cyclic group has a substituent, at least one of the substituents includes a hydrocarbon group having a bromine atom or a hydrocarbon group having an iodine atom, Yd⁰ represents a divalent linking group or a single bond, Yd⁰ is bonded to the alicyclic ring in the condensed cyclic group, M^{m+} represents an m-valent organic cation, m represents an integer of 1 or more

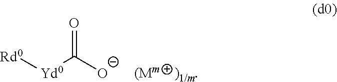

### TECHNICAL FIELD

The present invention relates to a resist composition, a resist pattern forming method, a compound, and an acid diffusion control agent.

Priority is claimed on Japanese Patent Application No. 2021-155753, filed on Sep. 24, 2021, the content of which is incorporated herein by reference.

### BACKGROUND ART

In recent years, in the production of semiconductor elements and liquid crystal display elements, advances in lithography techniques have led to rapid progress in the field of pattern fining. Typically, these pattern fining techniques involve shortening the wavelength (increasing the energy) of the light source for exposure.

Resist materials for use with these types of light sources for exposure require lithography characteristics such as a high resolution capable of reproducing a fine-sized pattern, and a high level of sensitivity to these types of light sources for exposure.

As a resist material that satisfies these requirements, a chemical amplification-type resist composition that contains a base material component that exhibits changed solubility in a developing solution under the action of an acid, and an acid generator component that generates an acid upon exposure has been used in the related art.

In the resist pattern formation, the behavior of an acid generated from an acid generator component upon exposure is considered as one factor that has a great influence on lithography characteristics.

On the other hand, a chemical amplification-type resist composition having both an acid generator component and an acid diffusion control agent that controls the diffusion of the acid generated from the acid generator component upon exposure has been proposed.

For example, Patent Document I discloses an acid generator and an acid diffusion control agent, which have a specific bulky structure (a bicyclooctane skeleton) of which an anion moiety mainly consists of a hydrocarbon and have relatively enhanced hydrophobicity. The means mainly adopted in the invention described in Patent Document 1 is to employ a compound having an anion moiety having relatively enhanced hydrophobicity as an acid generator, and it discloses that according to a resist composition containing the compound, it is possible to achieve high sensitivity in the resist pattern formation and form a resist pattern having a good shape with high resolution and reduced roughness.

### CITATION LIST

### [Patent Document]

### [Patent Document 1]

Japanese Unexamined Patent Application, First Publication No. 2018-92159

### SUMMARY OF INVENTION

### Technical Problem

With the further progress of lithography technology and resist pattern fining, for example, it is a goal to form a fine pattern of several tens of nanometers in lithography by an extreme ultraviolet rays (EUV) or an electron beam (EB). As the resist pattern size becomes smaller as described above, a resist composition that makes it possible to form a resist pattern having good critical dimension uniformity (CDU) of the pattern size while having a high sensitivity to a light source for exposure is required.

In addition, in the case of a light source for exposure, particularly EUV or EB, the number of photons involved in photosensitivity is small as compared with the case of an ArF excimer laser or a KrF excimer laser, and thus it is required to further improve the sensitivity of the resist composition.

However, in a resist composition containing an acid diffusion control agent, such as the one described above from Patent Document 1, although the uniformity of the acid diffusion control agent in the resist film can be enhanced due to the improvement of the hydrophobicity since the anion moiety has a polycyclic structure containing a bicyclooctane skeleton, there is room for further improvement in sensitivity.

The present invention has been made in consideration of the above circumstances, and an object of the present invention is to provide a resist composition which makes it possible to achieve high sensitivity and form a resist pattern having good CDU, a resist pattern forming method using the resist composition, a novel compound useful as an acid diffusion control agent of the resist composition, and an acid diffusion control agent using the compound.

### Solution to Problem

In order to achieve the above-described object, the present invention employs the following configurations.

That is, a first aspect of the present invention is a resist composition that generates an acid upon exposure and exhibits changed solubility in a developing solution under an action of the acid, the resist composition containing a base material component (A) that exhibits changed solubility in a developing solution under the action of the acid and an acid diffusion control agent component (D) that controls the diffusion of the acid generated upon exposure, in which the acid diffusion control agent component (D) contains a compound (D0) represented by General Formula (d0).

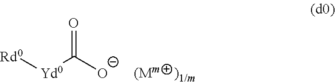

[In the formula, Rd⁰ represents a condensed cyclic group in which an aromatic ring and an alicyclic ring are condensed. The alicyclic ring in the condensed cyclic group has a substituent, where at least one of the substituents includes a hydrocarbon group having a bromine atom or a hydrocarbon group having an iodine atom. Yd⁰ represents a divalent linking group or a single bond. However, Yd⁰ is bonded to the alicyclic ring in the condensed cyclic group. M^{m+} represents an m-valent organic cation. m represents an integer of 1 or more.]

The second aspect according to the present invention is a resist pattern forming method, including a step of forming a resist film on a support using the resist composition according to the first aspect, a step of exposing the resist film, and a step of developing the exposed resist film to form a resist pattern.

A third aspect of the present invention is a compound represented by General Formula (d0).

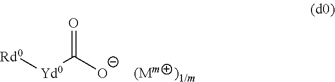

[In the formula, Rd⁰ represents a condensed cyclic group in which an aromatic ring and an alicyclic ring are condensed. The alicyclic ring in the condensed cyclic group has a substituent, where at least one of the substituents includes a hydrocarbon group having an iodine atom. Yd⁰ represents a divalent linking group or a single bond. However, Yd⁰ is bonded to the alicyclic ring in the condensed cyclic group. M^{m+} represents an m-valent organic cation. m represents an integer of 1 or more.]

A fourth aspect of the present invention is an acid diffusion control agent containing the compound according to the third aspect described above.

### Advantageous Effects of Invention

According to the present invention, it is possible to provide a resist composition which makes it possible to achieve high sensitivity and form a resist pattern having good CDU, a resist pattern forming method using the resist composition, a novel compound useful as an acid diffusion control agent of the resist composition, and an acid diffusion control agent using the compound.

### DESCRIPTION OF EMBODIMENTS

In the present specification and the scope of the present claims, the term “aliphatic” is a relative concept used with respect to “aromatic” and defines a group or compound that has no aromaticity.

The term “alkyl group” includes a monovalent saturated hydrocarbon group that is linear, branched, or cyclic unless otherwise specified. The same applies to the alkyl group of an alkoxy group.

The tern “alkylene group” includes a divalent saturated hydrocarbon group that is linear, branched, or cyclic unless otherwise specified.

Examples of the “halogen atom” include a fluorine atom, a chlorine atom, a bromine atom, and an iodine atom.

The term “constitutional unit” means a monomer unit (a monomeric unit) that contributes to the formation of a polymeric compound (a resin, a polymer, or a copolymer).

The expression “may have a substituent” includes both a case where a hydrogen atom (—H) is substituted with a monovalent group and a case where a methylene group (—CH₂—) is substituted with a divalent group.

The term “exposure” refers to a general concept that includes irradiation with any form of radiation.

The term “acid decomposable group” indicates a group having acid decomposability, in which at least a part of a bond in the structure of the acid decomposable group can be cleaved under the action of an acid.

Examples of the acid decomposable group having a polarity that is increased under the action of an acid include groups that are decomposed under the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected with an acid dissociable group (for example, a group in which a hydrogen atom of the OH-containing polar group has been protected with an acid dissociable group).

The term “acid dissociable group” indicates any one of (i) a group having acid dissociability, in which a bond between the acid dissociable group and an atom adjacent to the acid dissociable group can be cleaved under the action of an acid; and (ii) a group in which bonds are partially cleaved under the action of an acid, and then a decarboxylation reaction occurs, whereby the bond between the acid dissociable group and the atom adjacent to the acid dissociable group can be cleaved.

It is necessary that the acid dissociable group that constitutes the acid decomposable group be a group that exhibits a lower polarity than the polar group generated by the dissociation of the acid dissociable group. Thus, in a case where the acid dissociable group is dissociated under the action of an acid, a polar group that exhibits a higher polarity than the acid dissociable group is generated, thereby increasing the polarity. As a result of the above, the polarity of the total component (A1) is increased. By the increase in the polarity, the solubility in a developing solution relatively changes. The solubility in a developing solution is increased in a case where the developing solution is an alkali developing solution, whereas the solubility in a developing solution is decreased in a case where the developing solution is an organic developing solution.

The term “base material component” refers to an organic compound having a film-forming ability. The organic compounds used as the base material component are roughly classified into a non-polymer and a polymer. As the non-polymer, those having a molecular weight of 500 or more and less than 4,000 are usually used. Hereinafter, a “low molecular weight compound” refers to a non-polymer having a molecular weight of 500 or more and less than 4,000. As the polymer, those having a molecular weight of 1,000 or more are usually used. Hereinafter, “resin”, “polymeric compound”, or “polymer” refers to a polymer having a molecular weight of 1,000 or more. As the molecular weight of the polymer, a weight average molecular weight in terms of the polystyrene equivalent value determined by gel permeation chromatography (GPC) is used.

The phrase “constitutional unit derived from” means a constitutional unit that is formed by the cleavage of a multiple bond between carbon atoms, for example, an ethylenic double bond.

In the “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, examples of the acrylic acid ester include itaconic acid diester in which the substituent (R^{αx}) has been substituted with a substituent having an ester bond and α-hydroxyacryl ester in which the substituent (R^{αx}) has been substituted with a hydroxyalkyl group or a group obtained by modifying a hydroxyl group thereof. A carbon atom at the α-position of acrylic acid ester indicates the carbon atom bonded to the carbonyl group of acrylic acid unless otherwise specified.

Hereinafter, an acrylic acid ester in which the hydrogen atom bonded to the carbon atom at the α-position is substituted with a substituent is also referred to as an α-substituted acrylic acid ester.

The term “derivative” includes a compound in which the hydrogen atom at the α-position of the object compound has been substituted with another substituent such as an alkyl group or a halogenated alkyl group; and derivatives thereof. Examples of the derivatives thereof include a derivative in which the hydrogen atom of the hydroxyl group of the object compound in which the hydrogen atom at the α-position may be substituted with a substituent is substituted with an organic group; and a derivative in which a substituent other than a hydroxyl group is bonded to the object compound in which the hydrogen atom at the α-position may be substituted with a substituent. The α-position is the first carbon atom adjacent to the functional group unless otherwise specified.

Examples of the substituent that substitutes the hydrogen atom at the α-position of hydroxystyrene include those for R^{αx}.

In the present specification and the scope of the present claims, asymmetric carbon atoms may be present, and thus enantiomers or diastereomers may be present depending on the structures represented by the chemical formula. In that case, these isomers are represented by one chemical formula. These isomers may be used alone or in the form of a mixture.

### (Resist Composition)

The resist composition according to the present embodiment is a resist composition that generates an acid upon exposure and exhibits changed solubility in a developing solution under the action of the acid.

Such a resist composition contains a base material component (A) (hereinafter, also referred to as a “component (A)”) that exhibits changed solubility in a developing solution under the action of the acid, and an acid diffusion control agent component (D) that controls the diffusion of the acid generated upon exposure (hereinafter, also referred to as a “component (D)”).

In the resist composition according to the present embodiment, the component (A) may generate an acid upon exposure, or an additive component that is blended separately from the component (A) may generate an acid upon exposure.

Specifically, the resist composition according to the present embodiment may (1) further contain an acid generator component (B) (hereinafter, referred to as “component (B)”) that generates an acid upon exposure; (2) have a component (A) that generates an acid upon exposure; and (3) have a component (A) that generates an acid upon exposure and further contains component (B).

That is, in the cases of (2) and (3) described above, the component (A) becomes a “base material component which generates an acid upon exposure and has a solubility in a developing solution, which is changed by an action of the acid”. In a case where the component (A) is a base material component that generates an acid upon exposure and exhibits changed solubility in a developing solution under an action of the acid, it is preferable that the component (A1) described below be a resin that generates an acid upon exposure and exhibits changed solubility in a developing solution under an action of the acid. As such a resin, a polymeric compound having a constitutional unit that generates an acid upon exposure can be used. As the constitutional unit that generates an acid upon exposure, a known constitutional unit can be used.

Among the above, the resist composition according to the present embodiment is particularly preferably the case of the above (1). That is, the resist composition according to the present embodiment is preferably a resist composition that contains the component (A), the component (B), and the component (D).

In a case where a resist film is formed using the resist composition according to the present embodiment and the formed resist film is subjected to selective exposure, an acid is generated, for example, from the component (B) at exposed portions of the resist film, and the generated acid acts on the component (A) to change the solubility of the component (A) in a developing solution, whereas the solubility of the component (A) in a developing solution is not changed at unexposed portions, thereby generating the difference in solubility in the developing solution between exposed portions and unexposed portions of the resist film. Therefore, by subjecting the resist film to development, exposed portions of the resist film are dissolved and removed to form a positive-tone resist pattern in a case where the resist composition is a positive-tone type, whereas unexposed portions of the resist film are dissolved and removed to form a negative-tone resist pattern in a case where the resist composition is a negative-tone type.

In the present specification, a resist composition which forms a positive-tone resist pattern by dissolving and removing exposed portions of the resist film is called a positive-tone resist composition, and a resist composition which forms a negative-tone resist pattern by dissolving and removing unexposed portions of the resist film is called a negative-tone resist composition. The resist composition according to the present embodiment may be a positive-tone resist composition or a negative-tone resist composition. Further, in the formation of a resist pattern, the resist composition according to the present embodiment can be applied to an alkali developing process using an alkali developing solution in the developing treatment, or a solvent developing process using a developing solution containing an organic solvent (organic developing solution) in the developing treatment.

### <Component (A)>

In the resist composition according to the present embodiment, the component (A) preferably contains a resin component (A1) (hereinafter, also referred to as a “component (A1)”) that exhibits changed solubility in a developing solution under the action of an acid. In the alkali developing process and the solvent developing process, since the polarity of the base material component before and after the exposure is changed by using the component (A1), an excellent development contrast can be obtained.

As the component (A), another polymeric compound and/or a low molecular weight compound may be used in combination with the component (A l).

In the resist composition according to the present embodiment, the component (A) may be used alone or in a combination of two or more kinds thereof.

In regard to component (A1)

The component (A1) is a resin component that exhibits changed solubility in a developing solution under the action of an acid.

The component (A1) preferably has a constitutional unit (a1) that includes an acid decomposable group having a polarity that is increased under the action of an acid.

The component (A1) may have other constitutional units as necessary in addition to the constitutional unit (a1).

«Constitutional Unit (a1)»

The constitutional unit (a1) is a constitutional unit that contains an acid decomposable group having a polarity that is increased under the action of an acid.

Examples of the acid dissociable group include those which have been proposed so far as acid dissociable groups for the base resin for a chemical amplification-type resist composition.

Specific examples of the acid dissociable group of the base resin proposed for a chemical amplification-type resist composition include an “acetal-type acid dissociable group”, a “tertiary alkyl ester-type acid dissociable group”, and a “tertiary alkyloxycarbonyl acid dissociable group” described below.

### Acetal-Type Acid Dissociable Group:

Examples of the acid dissociable group for protecting a carboxy group or a hydroxyl group as a polar group include the acid dissociable group represented by General Formula (a1-r-1) shown below (hereinafter, also referred to as an “acetal-type acid dissociable group”).

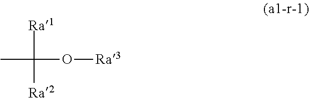

[In the formula, Ra′¹ and Ra′² each represent a hydrogen atom or an alkyl group. Ra′³ represents a hydrocarbon group, and Ra′³ may be bonded to any of Ra′¹ and Ra′² to form a ring.]

In General Formula (a1-r-1), it is preferable that at least one of Ra′¹ or Ra′² represent a hydrogen atom and more preferable that both Ra′¹ and Ra′² represent a hydrogen atom.

In a case where Ra′¹ or Ra′² represents an alkyl group, examples of the alkyl group include the same alkyl groups exemplified as the substituent which may be bonded to the carbon atom at the α-position in the description of the α-substituted acrylic acid ester. Among these, an alkyl group having 1 to 5 carbon atoms is preferable. Specific examples thereof preferably include a linear or branched alkyl group. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is preferable, and a methyl group is particularly preferable.

In General Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane preferably has 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, and tetracyclododecane.

In a case where the cyclic hydrocarbon group as Ra′³ is an aromatic hydrocarbon group, the aromatic hydrocarbon group is a hydrocarbon group having at least one aromatic ring.

The aromatic ring is not particularly limited as long as it is a cyclic conjugated system having (4n+2) π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms.

Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group bonded to the aromatic hydrocarbon ring or aromatic heterocyclic ring preferably has 1 to 4 carbon atoms, more preferably has 1 or 2 carbon atoms, and particularly preferably has 1 carbon atom.

The cyclic hydrocarbon group as Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents will also be collectively referred to as “Ra^{x5}”).

Here, R^{P1} represents a monovalent chain-like saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. Further, R^{PZ} represents a single bond, a chain-like divalent saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Here, some or all hydrogen atoms in the chain-like saturated hydrocarbon group, the aliphatic cyclic saturated hydrocarbon group, and the aromatic hydrocarbon group as R^{P1} and R^{P2} may be substituted with fluorine atoms. In the aliphatic cyclic hydrocarbon group, one or more of the above-described substituents may be included as a single kind, or one or more of the above-described substituents may be included as a plurality of kinds.

Examples of the monovalent chain-like saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7] dodecanyl group, and an adamantyl group.

Examples of the monovalent aromatic hydrocarbon group having 6 to 30 carbon atoms include a group obtained by removing one hydrogen atom from an aromatic hydrocarbon ring such as benzene, biphenyl, fluorene, naphthalene, anthracene, or phenanthrene.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

### Tertiary Alkyl Ester-Type Acid Dissociable Group:

Among the above polar groups, examples of the acid dissociable group for protecting the carboxy group include the acid dissociable group represented by General Formula (a1-r-2) shown below.

Among the acid dissociable groups represented by General Formula (a1-r-2), for convenience, a group which is constituted of alkyl groups is referred to as a “tertiary alkyl ester-type acid dissociable group”.

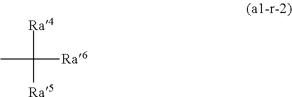

[In the formula, Ra′⁴ to Ra′⁶ each represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′4, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ independently represent a hydrocarbon group without being bonded to each other, suitable examples thereof include a group represented by General Formula (a1-r2-4).

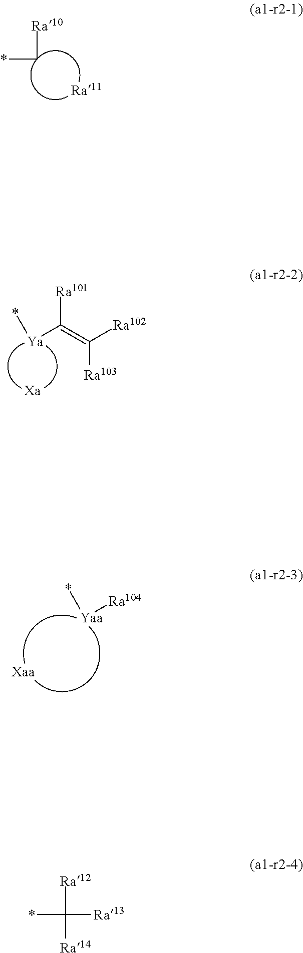

[In General Formula (a1-r2-1), Ra′¹⁰ represents a linear or branched alkyl group having 1 to 12 carbon atoms, some of which may be substituted with a halogen atom or a hetero atom-containing group. Ra′¹¹ represents a group that forms an aliphatic cyclic group together with the carbon atom to which Ra′¹⁰ has been bonded. In General Formula (a1-r2-2), Ya represents a carbon atom. Xa is a group that forms a cyclic hydrocarbon group together with Ya. Some or all hydrogen atoms contained in the cyclic hydrocarbon group may be substituted. Ra¹⁰¹ to Ra¹⁰³ each independently represent a hydrogen atom, a monovalent chain-like saturated hydrocarbon group having 1 to 10 carbon atoms, or a monovalent aliphatic cyclic saturated hydrocarbon group having 3 to 20 carbon atoms. Some or all hydrogen atoms contained in the chain-like saturated hydrocarbon group and the aliphatic cyclic saturated hydrocarbon group may be substituted. Two or more of Ra¹⁰¹ to Ra¹⁰³ may be bonded to each other to form a cyclic structure. In General Formula (a1-r2-3), Yaa represents a carbon atom. Xaa is a group that forms an aliphatic cyclic group together with Yaa. Ra¹⁰⁴ represents an aromatic hydrocarbon group which may have a substituent. In General Formula (a1-r2-4), Ra′¹² and Ra′¹³ each independently represent a monovalent chain-like saturated hydrocarbon group having 1 to 10 carbon atoms. Some or all hydrogen atoms contained in the chain-like saturated hydrocarbon group may be substituted. Ra′¹⁴ represents a hydrocarbon group which may have a substituent. * represents a bonding site.]

In General Formula (a1-r2-1), Ra′¹⁰ represents a linear or branched alkyl group having 1 to 12 carbon atoms, some of which may be substituted with a halogen atom or a hetero atom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a hetero atom-containing group. For example, some hydrogen atoms constituting the alkyl group may be substituted with a halogen atom or a hetero atom-containing group. Further, some carbon atoms (those in the methylene group or the like) constituting the alkyl group may be substituted with a hetero atom-containing group.

Examples of the hetero atom mentioned here include an oxygen atom, a sulfur atom, and a nitrogen atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In General Formula (a1-r2-1), preferred examples of Ra′¹¹ (a group that forms an aliphatic cyclic group together with the carbon atom to which Ra¹⁰ has been bonded) include the groups exemplified as the aliphatic hydrocarbon group (alicyclic hydrocarbon group) which is a monocyclic group or a polycyclic group as Ra′³ in General Formula (a1-r-1). Among them, it is preferably a monocyclic alicyclic hydrocarbon group, and specifically, it is more preferably a cyclopentyl group or a cyclohexyl group.

In General Formula (a1-r2-2), examples of the cyclic hydrocarbon group that is formed by Xa together with Ya include a group in which one or more hydrogen atoms have been further removed from the cyclic monovalent hydrocarbon group (aliphatic hydrocarbon group) as Ra′³ in General Formula (a1-r-1).

The cyclic hydrocarbon group that is formed by Xa together with Ya may have a substituent. Examples of the substituent include those exemplified as the substituents which may be contained in the cyclic hydrocarbon group as Ra′³.

In General Formula (a1-r2-2), examples of the monovalent chain-like saturated hydrocarbon group having 1 to 10 carbon atoms as Ra¹⁰¹ to Ra′¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2] octanyl group, a tricyclo[5.2.1.02,6] decanyl group, a tricyclo[3.3.1.13,7]decanyl group, a tetracyclo[6.2.1.13,6.02,7] dodecanyl group, or an adamantyl group. From the viewpoint of ease of synthesis, Ra′¹¹⁰ to Ra¹⁰³ are preferably a hydrogen atom or a monovalent chain-like saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by R¹⁰¹ to Ra¹⁰³ include the same substituents as those for Ra^{x}s.

Examples of the group having a carbon-carbon double bond generated by two or more of Ra¹⁰¹ to Ra¹⁰³ being bonded to each other to form a cyclic structure include a cyclopentenyl group, a cyclohexenyl group, a methylcyclopentenyl group, a methylcyclohexenyl group, a cyclopentylideneethenyl group, and a cyclohexylideneethenyl group. Among these, a cyclopentenyl group, a cyclohexenyl group, and a cyclopentylideneethenyl group are preferable from the viewpoint of easy synthesis.

In General Formula (a1-r2-3), as the aliphatic cyclic group that is formed by Xaa together with Yaa, the group exemplified as the aliphatic hydrocarbon group which is a monocyclic group or a polycyclic group as Ra′³ in General Formula (a1-r-1) is preferable.

In General Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent which may be contained in Ra¹⁰⁴ in General Formula (a1-r2-3) include a methyl group, an ethyl group, a propyl group, a hydroxyl group, a carboxyl group, a halogen atom, an alkoxy group (such as a methoxy group, an ethoxy group, a propoxy group, or a butoxy group), and an alkyloxycarbonyl group.

In General Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹ and Ra′¹³ include those exemplified as the monovalent chain-like saturated hydrocarbon group having 1 to 10 carbon atoms as Ra¹⁰¹ to Ra¹⁰³. Some or all hydrogen atoms contained in the chain-like saturated hydrocarbon group may be substituted.

Among them, Ra′¹² and Ra′³ are preferably a hydrogen atom or an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent thereof include the same substituents as those for Ra′.

In General Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be a polycyclic group or a monocyclic group.

The aliphatic hydrocarbon group which is a monocyclic group is preferably a group obtained by removing one hydrogen atom from a monocycloalkane. The monocycloalkane preferably has 3 to 6 carbon atoms, and specific examples thereof include cyclopentane and cyclohexane.

The aliphatic hydrocarbon group which is a polycyclic group is preferably a group obtained by removing one hydrogen atom from a polycycloalkane. The polycycloalkane preferably has 7 to 12 carbon atoms, and specific examples thereof include adamantane, norbornane, isobornane, tricyclodecane, and tetracyclododecane.

Examples of the aromatic hydrocarbon group as Ra′¹⁴ include the same groups as those for the aromatic hydrocarbon group as Ra¹⁰⁴. Among these, Ra′¹⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be contained in Ra′¹⁴ include the same groups as those for the substituent which may be contained in Ra¹⁰⁴.

In a case where Ra′¹⁴ in General Formula (a1-r2-4) represents a naphthyl group, the position bonded to the tertiary carbon atom in General Formula (aI-r2-4) may be the 1-position or the 2-position of the naphthyl group.

In a case where Ra′¹⁴ in General Formula (a1-r2-4) represents an anthryl group, the position bonded to the tertiary carbon atom in General Formula (a1-r2-4) may be the 1-position, the 2-position, or the 9-position of the anthryl group.

Specific examples of the group represented by General Formula (a1-r2-1) are shown below.

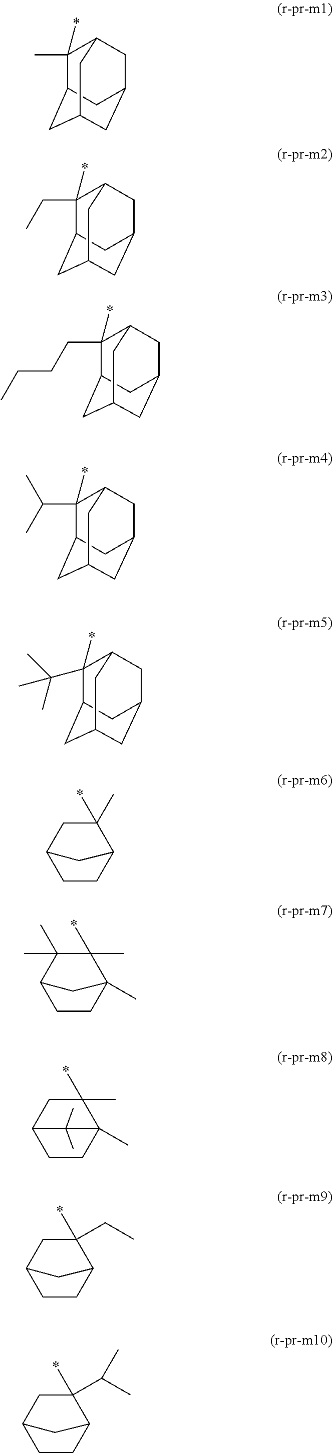

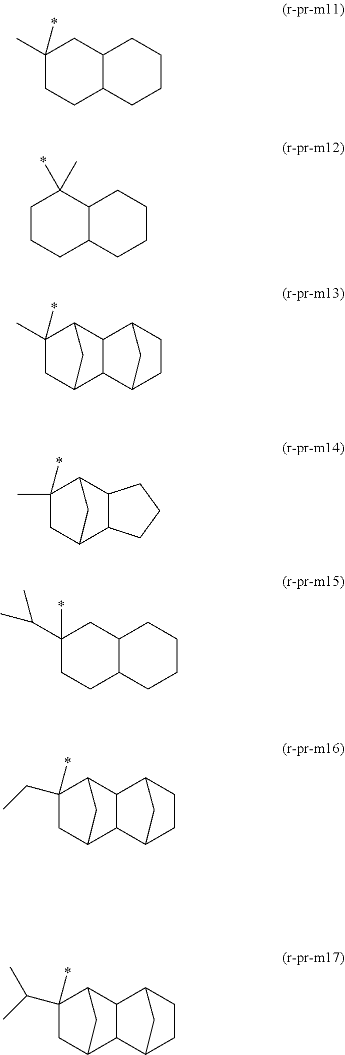

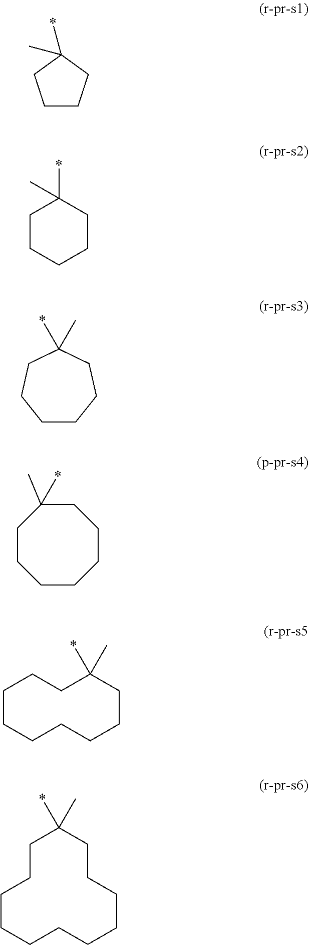

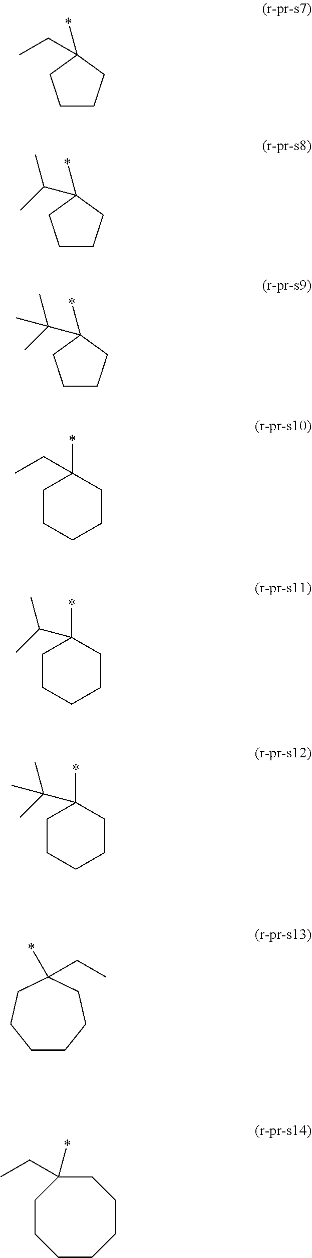

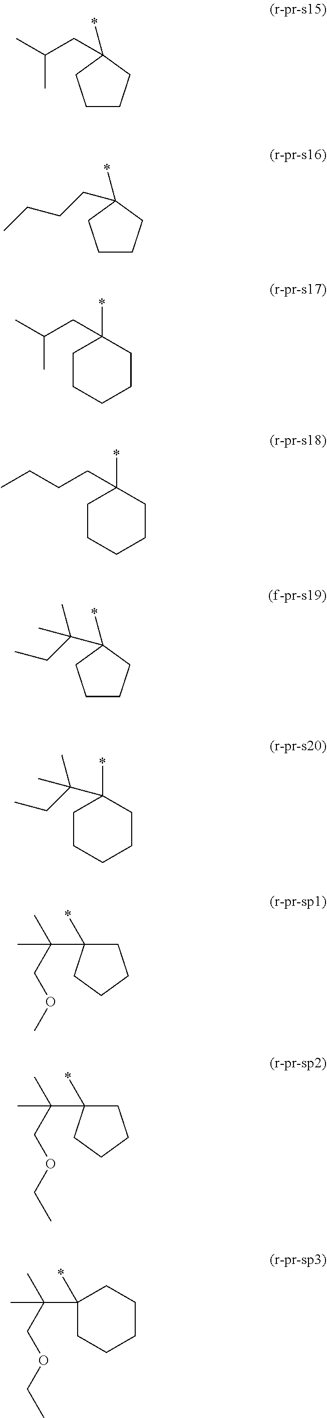

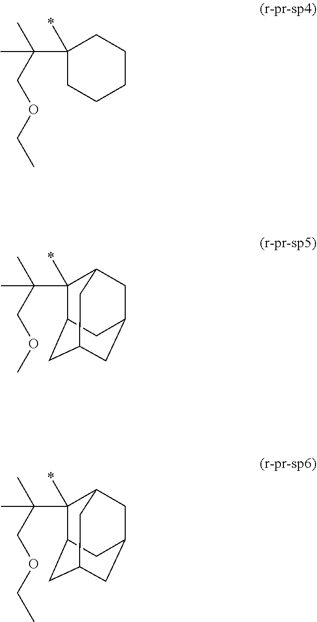

Specific examples of the group represented by General Formula (a1-r2-2) are shown below.

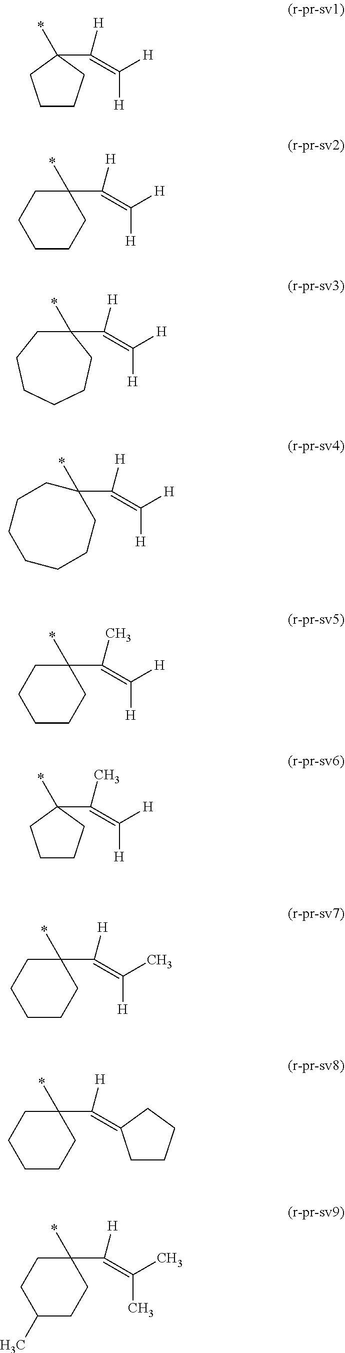

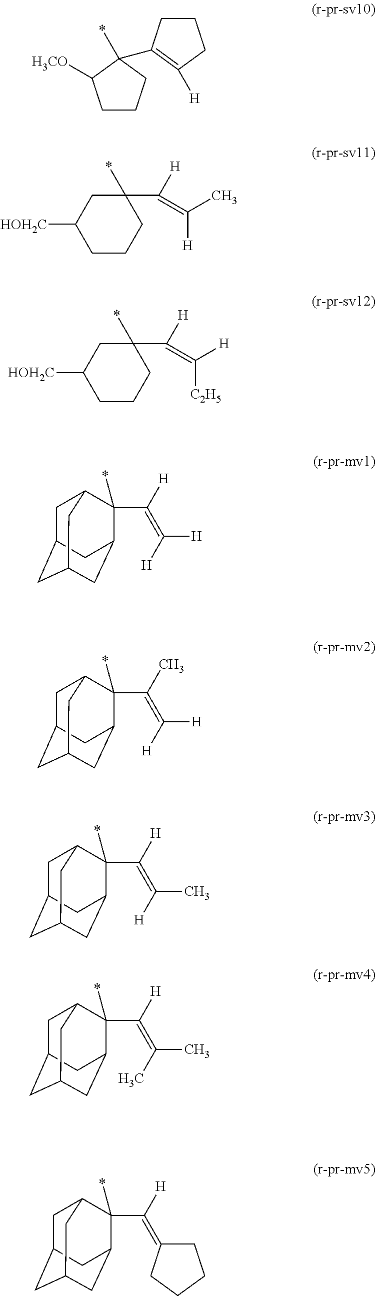

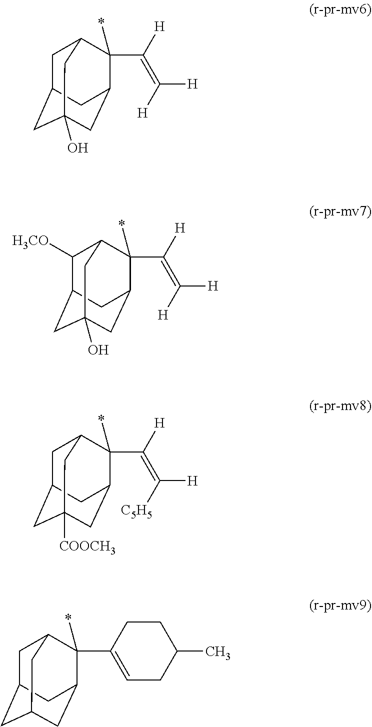

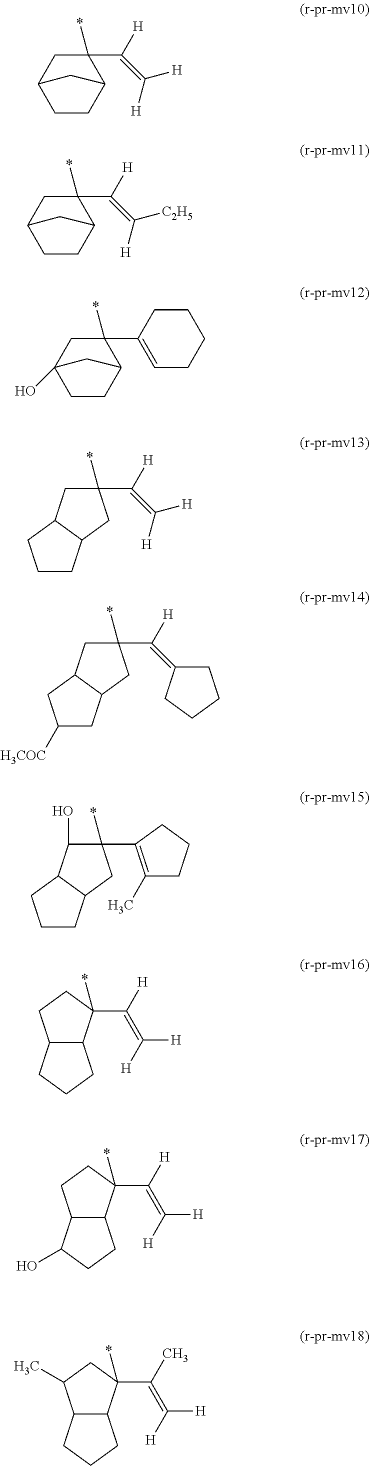

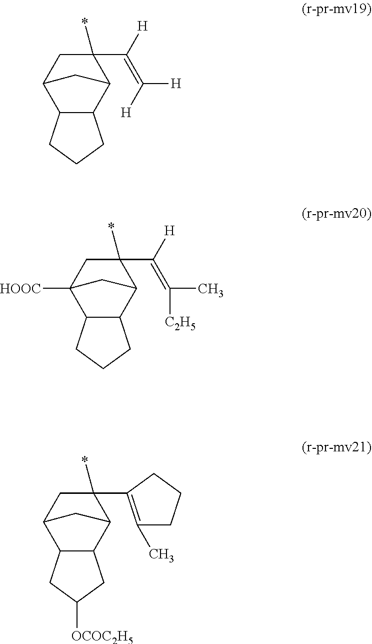

Specific examples of the group represented by General Formula (a1-r2-3) are shown below.

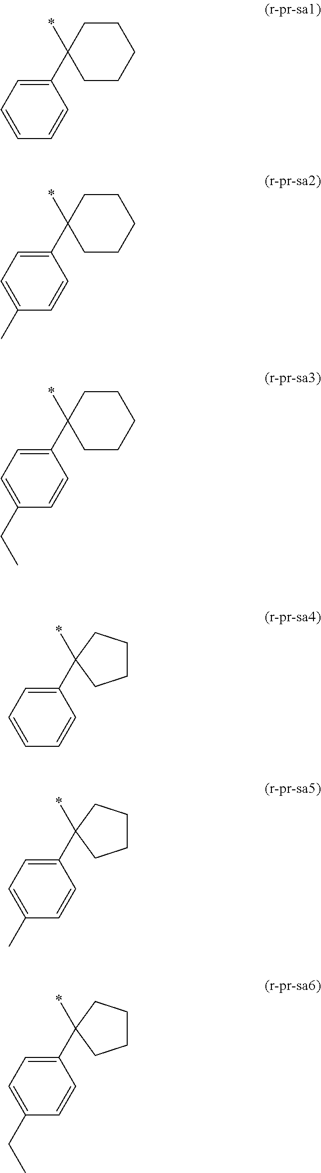

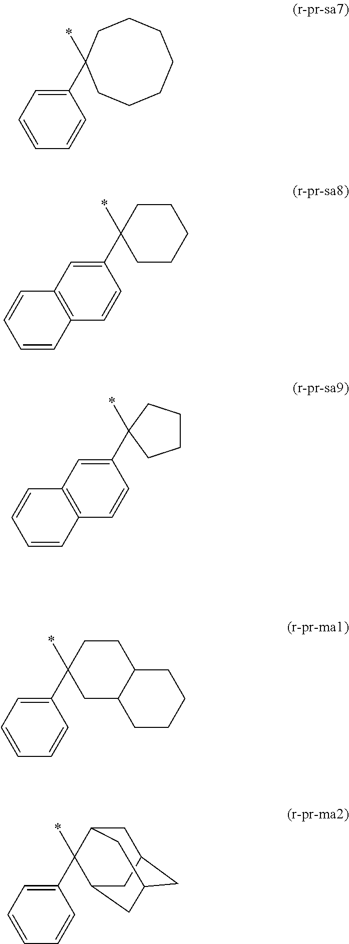

Specific examples of the group represented by General Formula (a1-r2-4) are shown below.

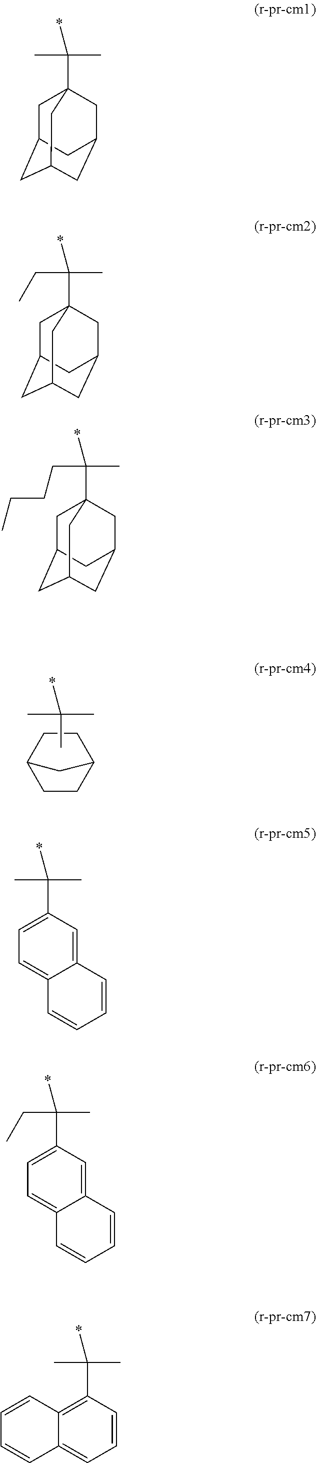

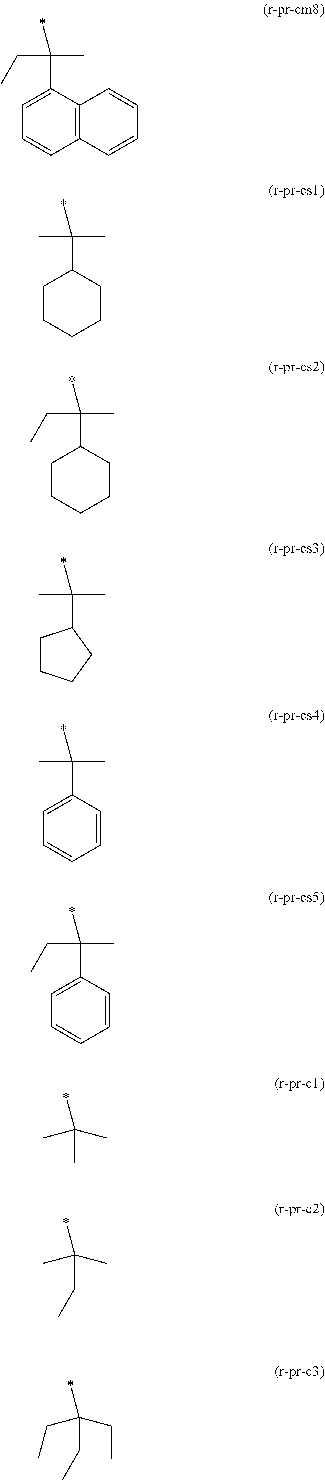

Tertiary alkyloxycarbonyl acid dissociable group:

Among the polar groups, examples of the acid dissociable group for protecting a hydroxyl group include an acid dissociable group (hereinafter, for convenience, also referred to as a “tertiary alkyloxycarbonyl acid dissociable group”) represented by General Formula (a1-r-3) shown below.

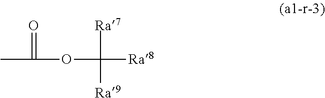

[In the formula, Ra′⁷ to Ra′⁹ each represent an alkyl group.]

In General Formula (a1-r-3), Ra′⁷ to Ra′⁹ are each preferably an alkyl group having 1 to 5 carbon atoms and more preferably an alkyl group having 1 to 3 carbon atoms.

Further, the total number of carbon atoms in each of the alkyl groups is preferably in a range of 3 to 7, more preferably in a range of 3 to 5, and most preferably 3 or 4.

Examples of the constitutional unit (a1) include a constitutional unit derived from an acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent including the above-described acid decomposable group; and a constitutional unit in which at least some hydrogen atoms in —C(═O)—OH of a constitutional unit derived from vinylbenzoic acid or a vinylbenzoic acid derivative are protected by a substituent including the above-described acid decomposable group.

Among the above, the constitutional unit (a1) is preferably a constitutional unit derived from an acrylic acid ester in which the hydrogen atom bonded to the carbon atom at the α-position may be substituted with a substituent.

Preferred specific examples of such a constitutional unit (a1) include constitutional units represented by General Formula (a1-1) or (a1-2).

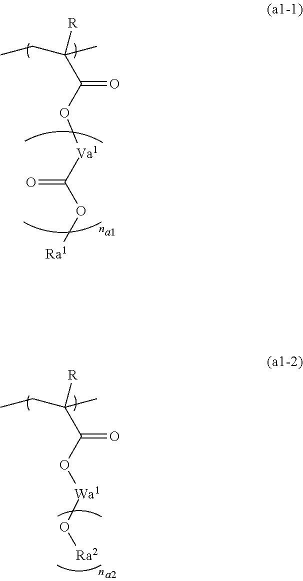

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐi represents an integer in a range of 0 to 2. Ra¹ represents an acid dissociable group represented by General Formula (a1-r-1) or (a1-r-2). Wa¹ represents a (nₐ₂+1)-valent hydrocarbon group, nₐ₂ represents an integer in a range of 1 to 3, and Ra² represents an acid dissociable group represented by General Formula (a1-r-1) or (a1-r-3).]

In General Formula (a1-i), the alkyl group having 1 to 5 carbon atoms as R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with a halogen atom. The halogen atom is particularly preferably a fluorine atom.

As R, a hydrogen atom, an alkyl group having 1 to 5 carbon atoms, or a fluorinated alkyl group having 1 to 5 carbon atoms is preferable, and a hydrogen atom or a methyl group is most preferable in terms of industrial availability.

In General Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group be saturated.

Specific examples of the aliphatic hydrocarbon group include a linear or branched aliphatic hydrocarbon group, and an aliphatic hydrocarbon group containing a ring in the structure thereof.

The linear aliphatic hydrocarbon group described above preferably has 1 to 10 carbon atoms, more preferably has 1 to 6 carbon atoms, still more preferably has 1 to 4 carbon atoms, and most preferably has 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group described above preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Examples of the aliphatic hydrocarbon group containing a ring in the structure thereof include an alicyclic hydrocarbon group (a group obtained by removing two hydrogen atoms from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the linear or branched aliphatic hydrocarbon group. Examples of the linear or branched aliphatic hydrocarbon group include the same one as the above-described linear aliphatic hydrocarbon group or the above-described branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group obtained by removing two hydrogen atoms from a monocycloalkane. The monocycloalkane preferably has 3 to 6 carbon atoms, and specific examples thereof include cyclopentane and cyclohexane. The polycyclic alicyclic hydrocarbon group is preferably a group obtained by removing two hydrogen atoms from a polycycloalkane, and the polycycloalkane is preferably a group having 7 to 12 carbon atoms. Specific examples of the polycycloalkane include adamantane, norbornane, isobornane, tricyclodecane, and tetracyclododecane.

The aromatic hydrocarbon group as the divalent hydrocarbon group represented by Va¹ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic hydrocarbon group include a group obtained by removing two hydrogen atoms from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group obtained by further removing one hydrogen atom from an aryl group in arylalkyl groups such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) preferably has 1 to 4 carbon atoms, more preferably has 1 or 2 carbon atoms, and particularly preferably has 1 carbon atom.

In General Formula (a1-1), Ra^{t} represents an acid dissociable group represented by General Formula (a1-r-1) or (a1-r-2).

In General Formula (a1-2), the (nₐ₂+1)-valent hydrocarbon group as Wai may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group be saturated. Examples of the aliphatic hydrocarbon group include a linear or branched aliphatic hydrocarbon group, an aliphatic hydrocarbon group containing a ring in the structure thereof, and a combination of the linear or branched aliphatic hydrocarbon group and the aliphatic hydrocarbon group containing a ring in the structure thereof. The valency of nₐ₂+1 is preferably divalent to tetravalent and more preferably divalent or trivalent.

In the Formula (a1-2), Ra² represents an acid dissociable group represented by General Formula (a1-r-1) or (a1-r-3).

Specific examples of the constitutional unit represented by General Formula (a1-1) are shown below. In the formulae shown below, R^{a} represents a hydrogen atom, a methyl group, or a trifluoromethyl group.

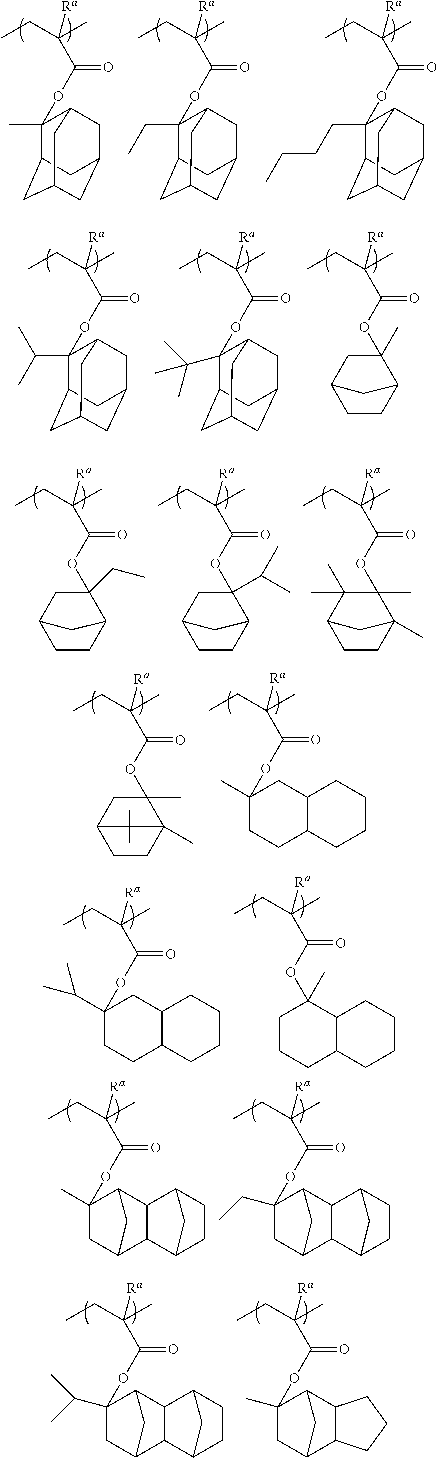

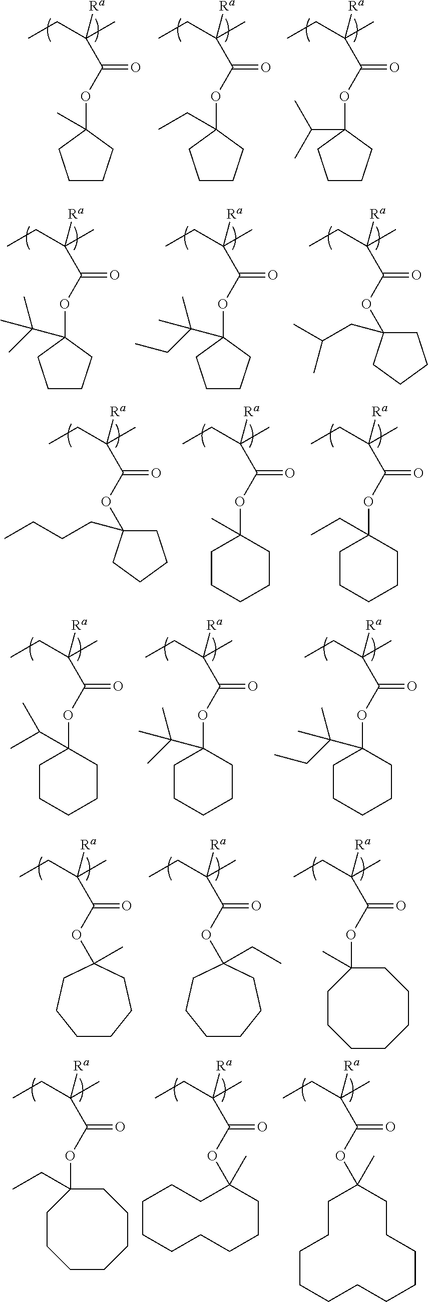

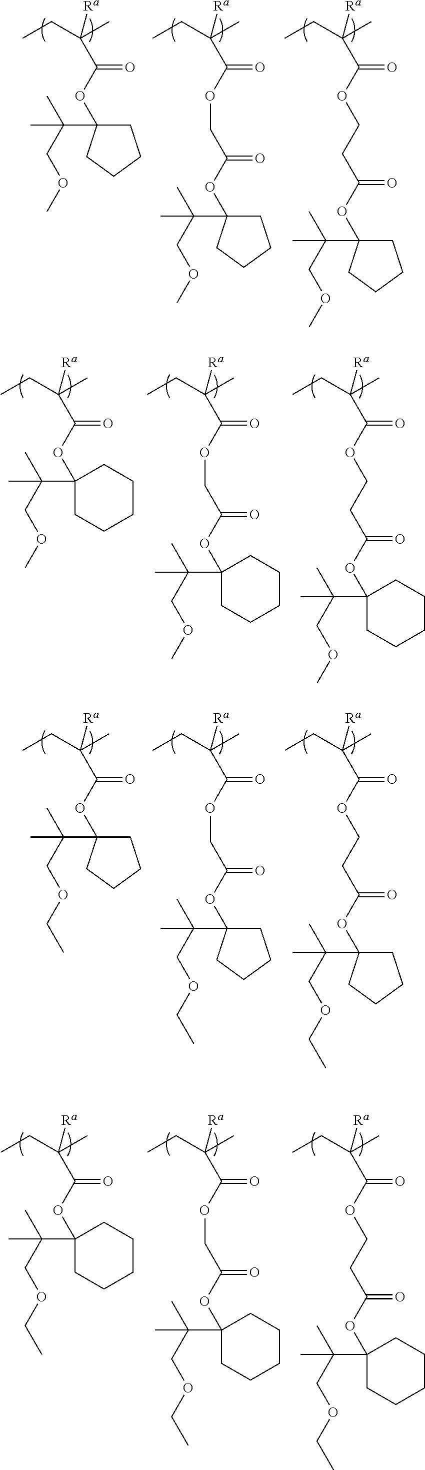

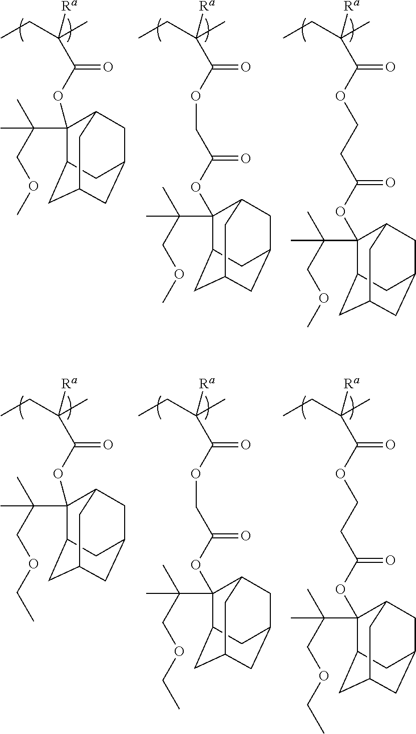

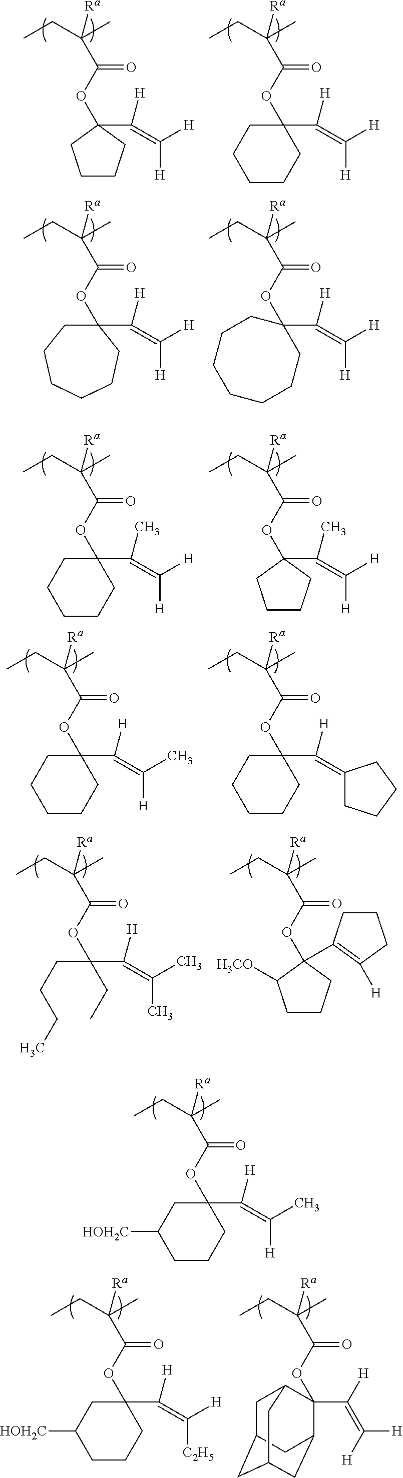

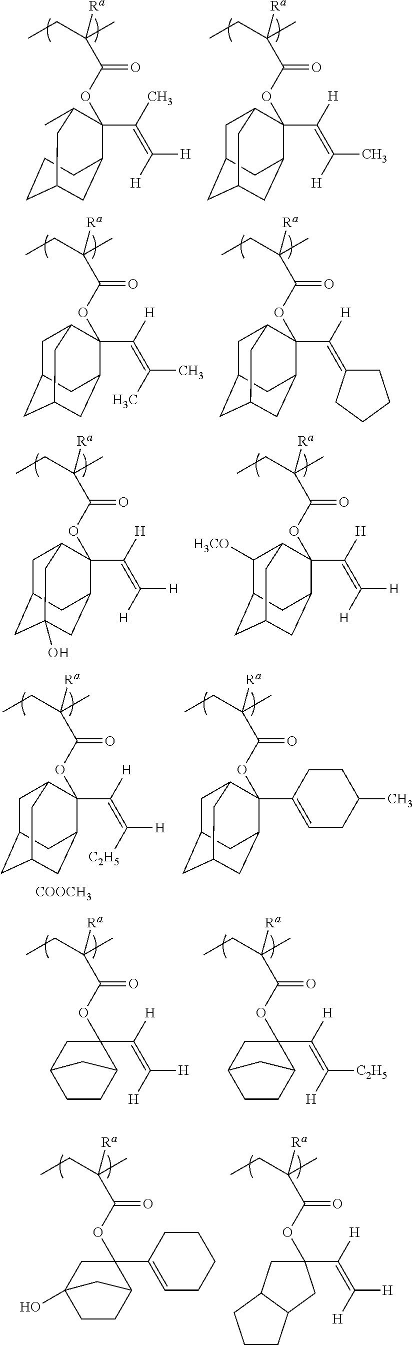

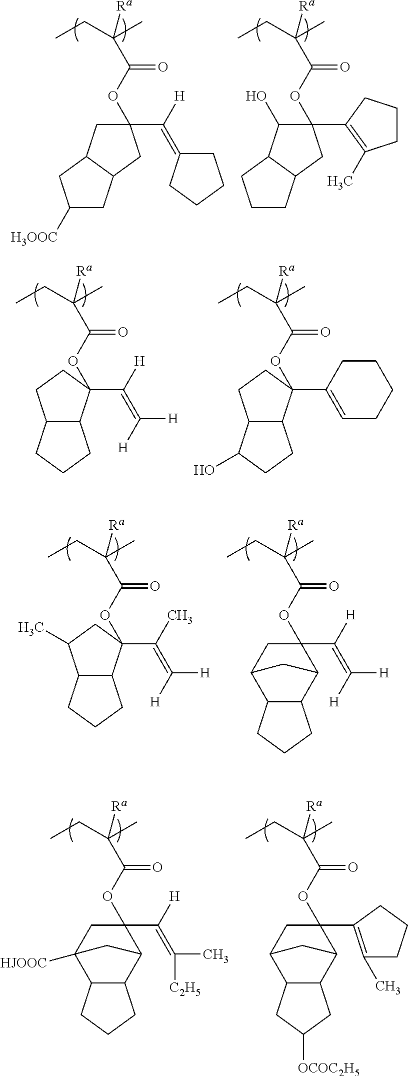

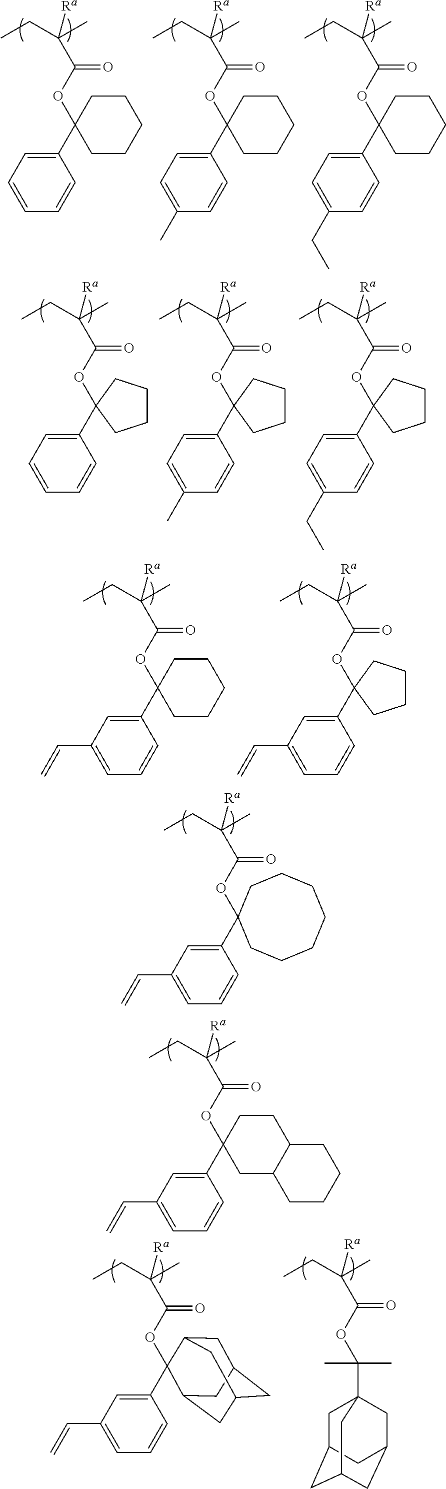

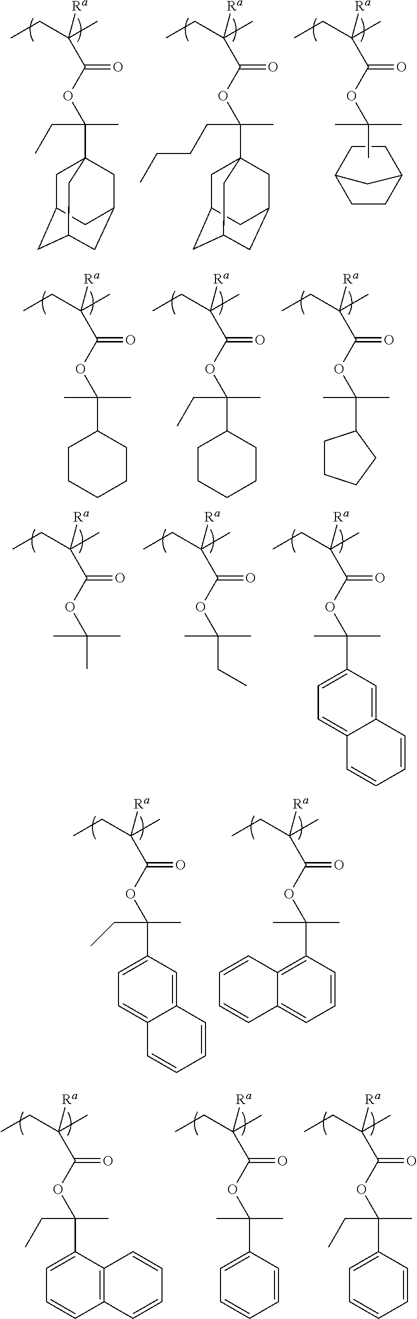

The constitutional unit (a1) contained in the component (A1) may be one kind or may be two or more kinds.

The constitutional unit (a1) is more preferably a constitutional unit represented by General Formula (a1-1) so that lithography characteristics (sensitivity, shape, and the like) depending on an electron beam or EUV can be more easily increased. Among these, the constitutional unit (a1) particularly preferably includes a constitutional unit represented by General Formula (a1-1-1) shown below.

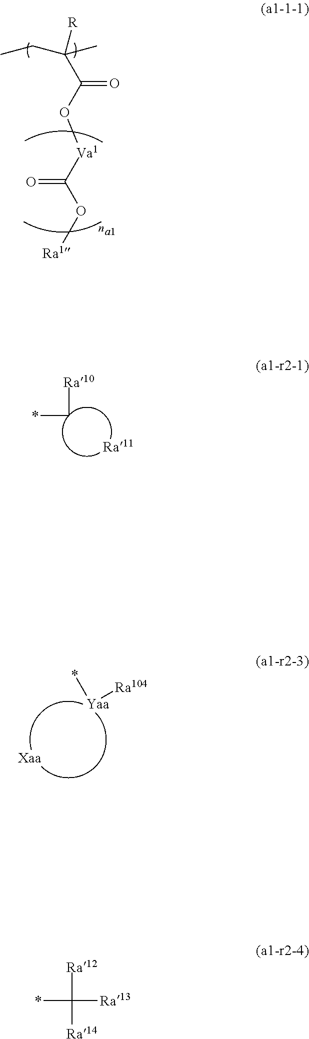

[In the formulae, Ra^{1″} represents an acid dissociable group represented by General Formula (a1-r2-1), (a1-r2-3), or (a1-r2-4). * represents a bonding site.]

R, Va¹, and nₐi in General Formula (a1-1-1) have the same definitions as R, Va¹, and nₐj in General Formula (a1-1), respectively.

The description for the acid dissociable group represented by General Formula (a1-r2-1), (a1-r2-3), or (a1-r2-4) is as described above. Among them, it is preferable to select a group in which the acid dissociable group is a cyclic group due to the fact that the reactivity can be increased, which is suitable for EB or EUV.

In General Formula (a1-1-1), it is preferable that Ra¹″ represent an acid dissociable group represented by General Formula (a1-r2-1) among the examples described above.

The proportion of the constitutional unit (a1) in the component (A1) is preferably in a range of 5% to 95% by mole, more preferably in a range of 10% to 90% by mole, still more preferably in a range of 30% to 70% by mole, and particularly preferably in a range of 40% to 60% by mole, with respect to the total (100% by mole) of all constitutional units constituting the component (A1).

In a case where the proportion of the constitutional unit (a1) is equal to or larger than the lower limit value of the above-described preferred range, lithography characteristics such as sensitivity, CDU, resolution, and roughness amelioration are improved. On the other hand, in a case where the proportion is equal to or smaller than the upper limit value of the above-described preferred range, balance with other constitutional units can be obtained, and various lithography characteristics are improved.

### Other Constitutional Units»

The component (A1) may have other constitutional units as necessary in addition to the constitutional unit (a1) described above.

Examples of the other constitutional units include a constitutional unit (a10) represented by General Formula (a10-1) described later; a constitutional unit (a2) containing a lactone-containing cyclic group; and a constitutional unit (a8) derived from a compound represented by General Formula (a8-1) described later.

In regard to constitutional unit (a10): The constitutional unit (a10) is a constitutional unit represented by General Formula (a10-1).

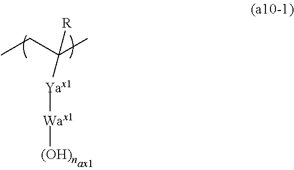

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or more.]

In General Formula (a10-1), R represents a hydrogen atom, an alkyl group having 1 to 5 carbon atoms, or a halogenated alkyl group having 1 to 5 carbon atoms. R is preferably a hydrogen atom, an alkyl group having 1 to 5 carbon atoms, or a fluorinated alkyl group having 1 to 5 carbon atoms, and in terms of industrial availability, R is more preferably a hydrogen atom, a methyl group, or a trifluoromethyl group, still more preferably a hydrogen atom or a methyl group, and particularly preferably a hydrogen atom.

In General Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a hetero atom.

Ya^{x1} is preferably a single bond, an ester bond [—C(═O)—O—, —O—C(═O)—], an ether bond (—O—), a linear or branched alkylene group, or a combination thereof, and more preferably a single bond or an ester bond [—C(═O)—O—, —O—C(═O)—].

In the Formula (a10-1), Wa^{x1} represents an aromatic hydrocarbon group which may have a substituent.

Examples of the aromatic hydrocarbon group as Wa^{x1} include a group in which (nₐₓ₁+1) hydrogen atoms have been removed from an aromatic ring which may have a substituent. Here, the aromatic ring is not particularly limited as long as it is a cyclic conjugated system having (4n+2) z electrons. The aromatic ring preferably has 5 to 30 carbon atoms, more preferably has 5 to 20 carbon atoms, still more preferably has 6 to 15 carbon atoms, and particularly preferably has 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound having an aromatic ring (for example, biphenyl or fluorene) which may have two or more substituents.

Among the examples, Wa^{x1} is preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may or may not have a substituent. Examples of the substituent include an alkyl group, an alkoxy group, a halogen atom, and a halogenated alkyl group. Examples of the alkyl group, the alkoxy group, the halogen atom, and the halogenated alkyl group as the substituent include those exemplified as the substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} have no substituent.

In General Formula (a10-1), nₐₓ₁ represents an integer of 1 or more, preferably an integer in a range of 1 to 10, more preferably an integer in a range of 1 to 5, still more preferably 1, 2, or 3, and particularly preferably 1 or 2.

Specific examples of the constitutional unit (a10) represented by General Formula (a10-1) are shown below.

In the formulae shown below, R^{α} represents a hydrogen atom, a methyl group, or a trifluoromethyl group.

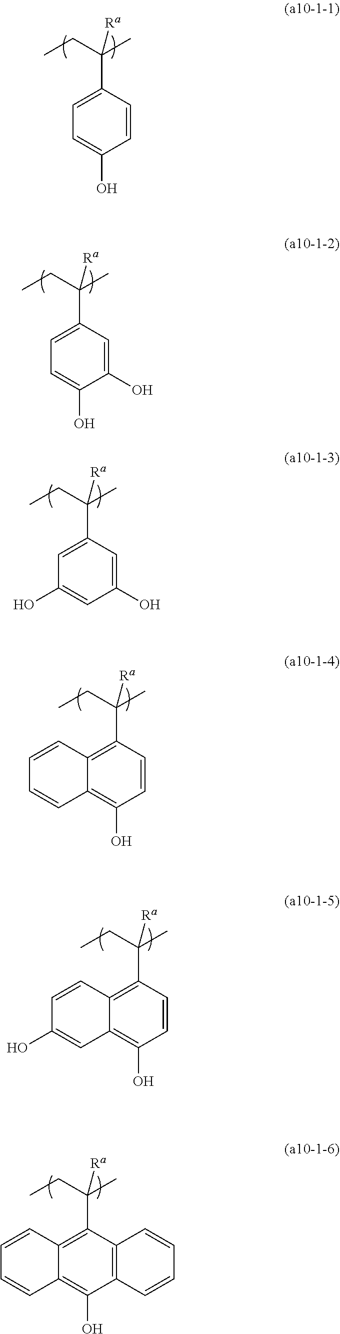

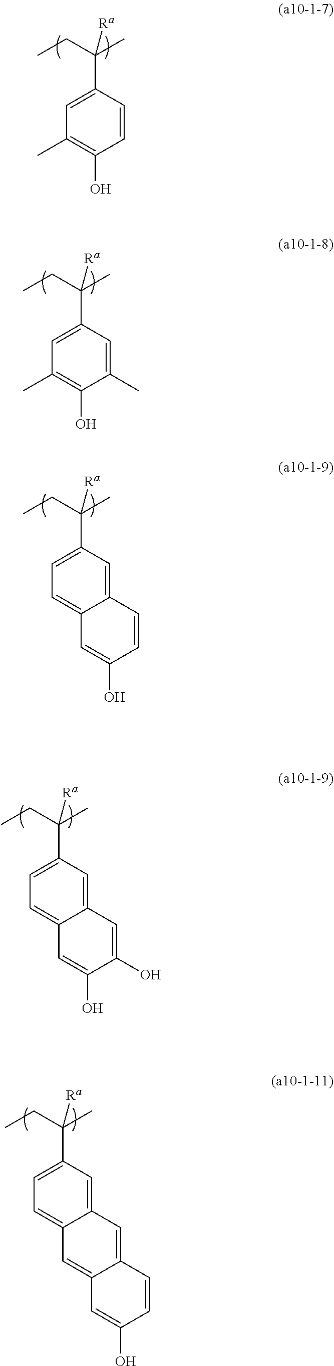

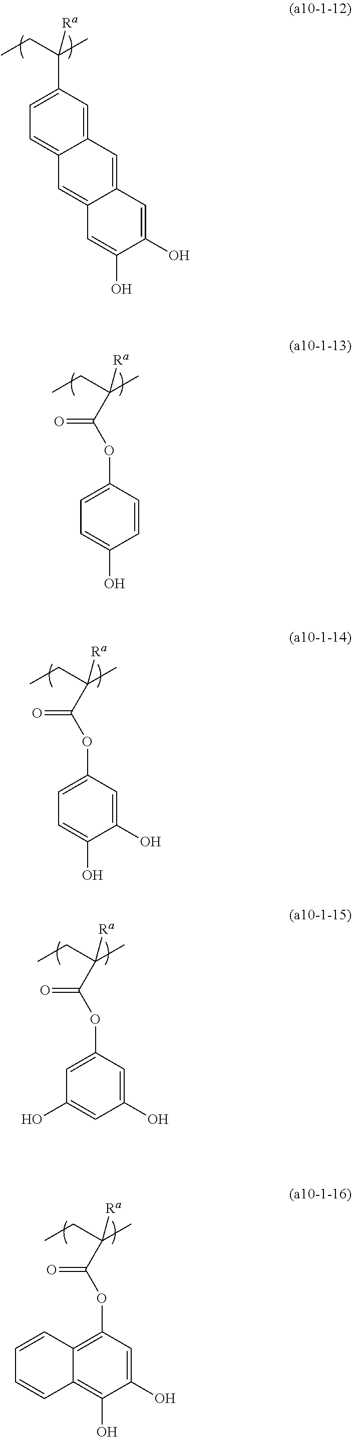

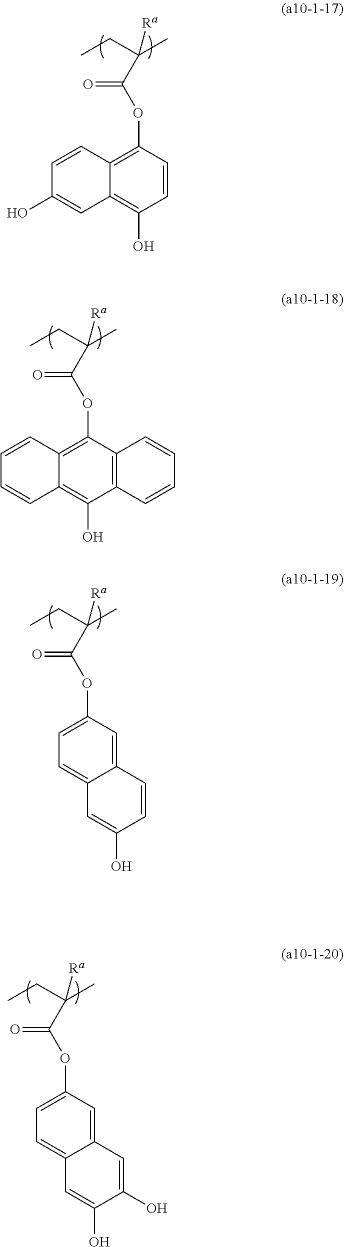

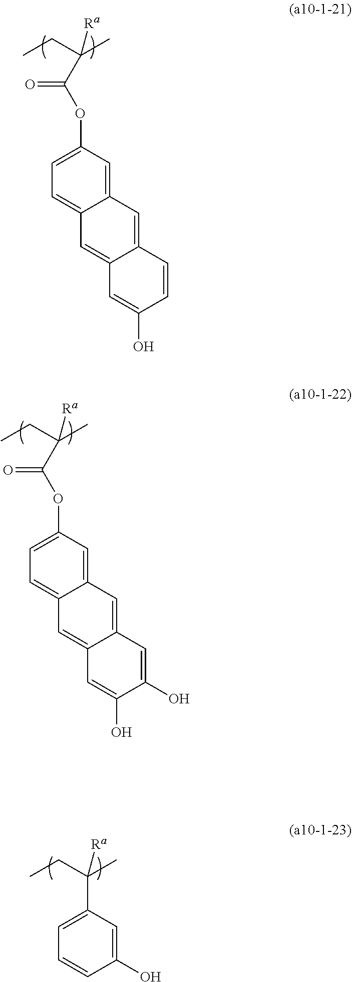

The constitutional unit (a10) contained in the component (A1) may be one kind or may be two or more kinds.

In a case where the component (A1) has the constitutional unit (a10), the proportion of the constitutional unit (a10) in the component (A1) is preferably in a range of 5% to 95% by mole, more preferably in a range of 10% to 90% by mole, still more preferably in a range of 30% to 70% by mole, and particularly preferably in a range of 40% to 60% by mole, with respect to the total (100% by mole) of all constitutional units constituting the component (A1).

In a case where the proportion of the constitutional unit (a10) is equal to or larger than the lower limit value, the sensitivity can be more easily increased. On the other hand, in a case where the proportion thereof is equal to or smaller than the upper limit value, balance with other constitutional units is obtained easily.

### In Regard to Constitutional Unit (a2):

The component (A l) may further have, in addition to the constitutional unit (a1), a constitutional unit (a2) (however, a constitutional unit corresponding to the constitutional unit (a1) is excluded) that further contains a lactone-containing cyclic group.

In a case where the component (A1) is used for forming a resist film, the lactone-containing cyclic group in the constitutional unit (a2) is effective for improving the adhesiveness of the resist film to the substrate. Further, due to having the constitutional unit (a2), lithography characteristics and the like can be improved, for example, by the effects obtained by appropriately adjusting the acid diffusion length, increasing the adhesiveness of the resist film to the substrate, and appropriately adjusting the solubility during development.

The term “lactone-containing cyclic group” indicates a cyclic group that contains a ring (lactone ring) containing a —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group for the constitutional unit (a2) is not particularly limited, and any lactone-containing cyclic group may be used. Specific examples thereof include groups represented by each of General Formulae (a2-r-1) to (a2-r-7) shown below.

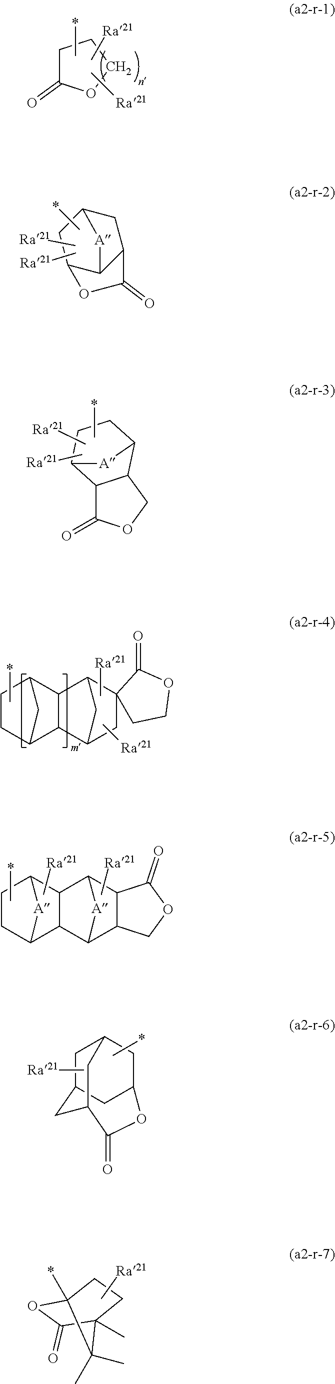

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, or a lactone-containing cyclic group; A″ represents an oxygen atom, a sulfur atom, or an alkylene group having 1 to 5 carbon atoms, which may contain an oxygen atom (—O—) or a sulfur atom (—S—); and n′ represents an integer in a range of 0 to 2, and m′ is 0 or 1. * represents a bonding site.]

In General Formulae (a2-r-1) to (a2-r-7), it is preferable that the alkyl group as Ra′²¹ be an alkyl group having 1 to 6 carbon atoms. The alkyl group is preferably a linear alkyl group or a branched alkyl group. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ be an alkoxy group having 1 to 6 carbon atoms. Further, the alkoxy group is preferably a linear or branched alkoxy group. Specific examples of the alkoxy groups include a group formed by linking the above-described alkyl group exemplified as the alkyl group represented by Ra′²¹ to an oxygen atom (—O—).

The halogen atom as Ra′²¹ is preferably a fluorine atom.

Examples of the halogenated alkyl group as Ra′²¹ include groups in which some or all hydrogen atoms in the alkyl group as Ra′²¹ have been substituted with the halogen atoms. The halogenated alkyl group is preferably a fluorinated alkyl group and particularly preferably a perfluoroalkyl group.

In —COOR″ and —OC(═O)R″ as Ra′²¹, any R″ represents a hydrogen atom, an alkyl group, or a lactone-containing cyclic group.

The alkyl group as R″ may be linear, branched, or cyclic, and preferably has 1 to 15 carbon atoms.

In a case where R″ represents a linear or branched alkyl group, it is preferably an alkyl group having 1 to 10 carbon atoms, more preferably an alkyl group having 1 to 5 carbon atoms, and particularly preferably a methyl group or an ethyl group.

In a case where R″ represents a cyclic alkyl group, the cyclic alkyl group preferably has 3 to 15 carbon atoms, more preferably has 4 to 12 carbon atoms, and most preferably has 5 to 10 carbon atoms. Specific examples thereof include a group obtained by removing one or more hydrogen atoms from a monocycloalkane, which may or may not be substituted with a fluorine atom or a fluorinated alkyl group; and a group obtained by removing one or more hydrogen atoms from a polycycloalkane such as bicycloalkane, tricycloalkane, or tetracycloalkane. More specific examples thereof include a group obtained by removing one or more hydrogen atoms from a monocycloalkane such as cyclopentane or cyclohexane; and a group obtained by removing one or more hydrogen atoms from a polycycloalkane such as adamantane, norbornane, isobornane, tricyclodecane, or tetracyclododecane.

Examples of the lactone-containing cyclic group as R″ include the same ones as the groups represented by each of General Formulae (a2-r-1) to (a2-r-7). As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that Ra′²¹'s each independently represent a hydrogen atom or a cyano group.

In General Formulae (a2-r-2), (a2-r-3), and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, examples of which include —O—CH₂—, —CH₂—O—CH₂—, —S—CH₂—, and —CH₂—S—CH₂—. A″ is preferably an alkylene group having 1 to 5 carbon atoms or —O—, more preferably an alkylene group having 1 to 5 carbon atoms, and most preferably a methylene group.

Specific examples of the groups represented by each of General Formulae (a2-r-1) to (a2-r-7) are shown below.

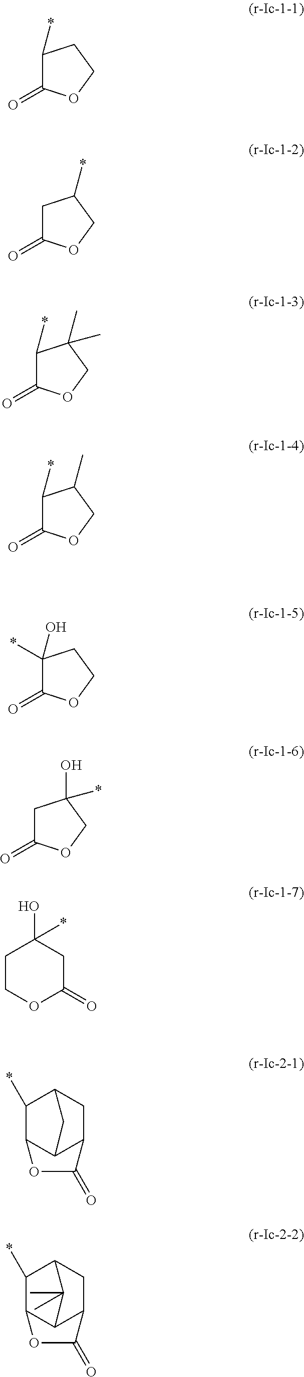

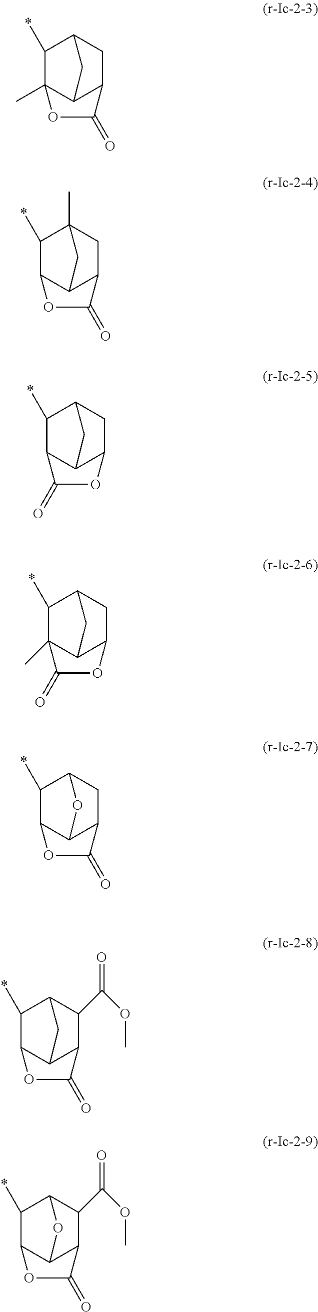

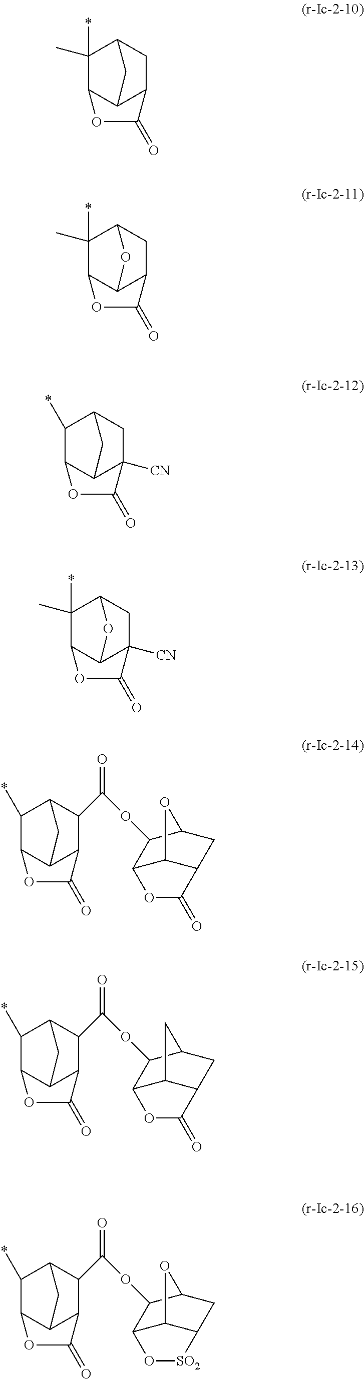

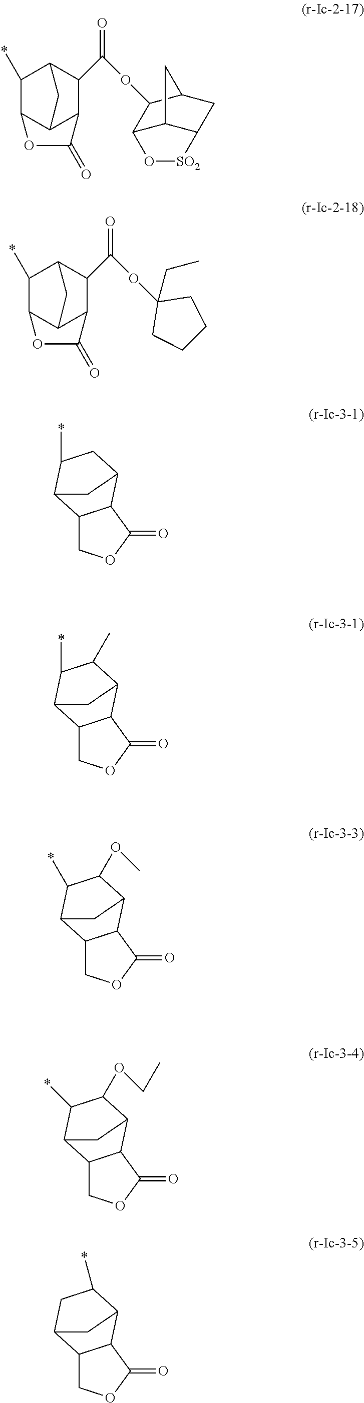

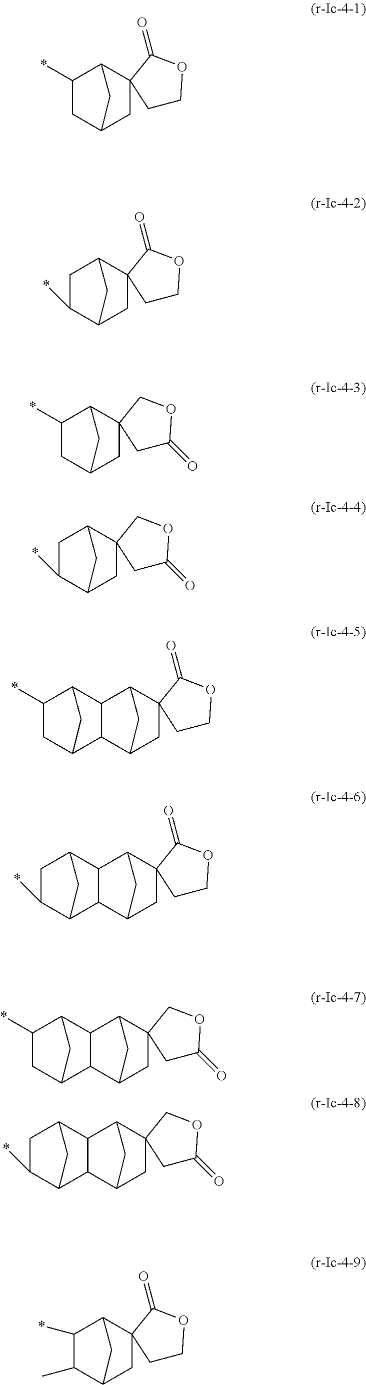

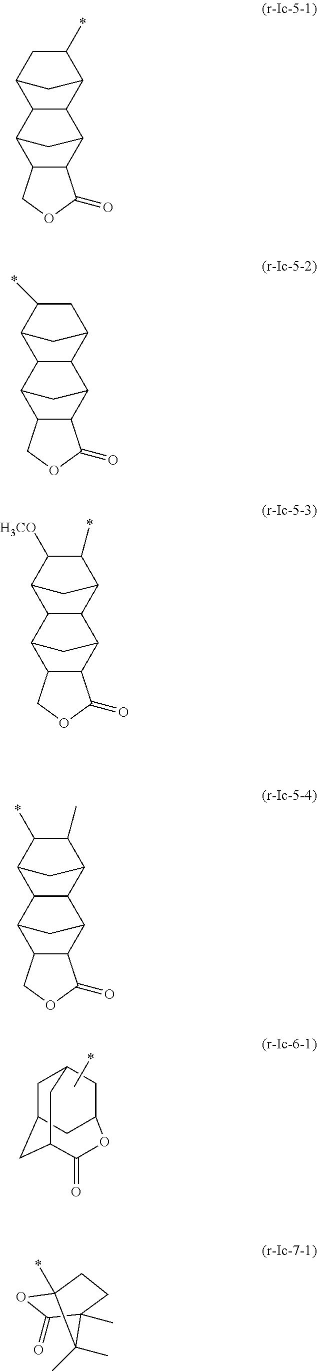

Among them, the constitutional unit (a2) is preferably a constitutional unit derived from an acrylic acid ester in which the hydrogen atom bonded to the carbon atom at the α-position may be substituted with a substituent.

The constitutional unit (a2) is preferably a constitutional unit represented by General Formula (a2-1).

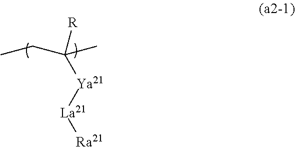

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In General Formula (a2-1), R has the same definition as described above. R is preferably a hydrogen atom, an alkyl group having 1 to 5 carbon atoms, or a fluorinated alkyl group having 1 to 5 carbon atoms, and particularly preferably a hydrogen atom or a methyl group in terms of industrial availability.

In General Formula (a2-1), the divalent linking group as Ya²¹ is not particularly limited, and suitable examples thereof include a divalent hydrocarbon group which may have a substituent and a divalent linking group having a hetero atom.

Ya²¹ is preferably a single bond, an ester bond [—C(═O)—O—], an ether bond (—O—), a linear or branched alkylene group, or a combination thereof.

In General Formula (a2-1), Ra²¹ represents a lactone-containing cyclic group.

Suitable examples of the lactone-containing cyclic group as Ra²¹ include the groups represented by each of General Formulae (a2-r-1) to (a2-r-7) described above.

Among them, a group represented by any of General Formulae (a2-r-1), (a2-r-2), or (a2-r-6) is preferable, and the group represented by General Formula (a2-r-2) is more preferable. Specifically, any one of the groups represented by each of Chemical Formulae (r-1c-1-1) to (r-1c-1-7), (r-1c-2-1) to (r-1c-2-18), and (r-1c-6-1) is preferable, any one of the groups represented by each of Chemical Formulae (r-1c-2-1) to (r-1c-2-18) is more preferable, and any one of the groups represented by each of Chemical Formula (r-1c-2-1) and (r-le-2-12) is still more preferable.

The constitutional unit (a2) contained in the component (A1) may be one kind or may be two or more kinds.

In a case where the component (A1) has the constitutional unit (a2), the proportion of the constitutional unit (a2) is preferably in a range of 5% to 60% by mole, more preferably in a range of 10% to 60% by mole, still more preferably in a range of 20% to 60% by mole, and particularly preferably in a range of 30% to 60% by mole with respect to the total (100% by mole) of all constitutional units constituting the component (A1).

In a case where the proportion of the constitutional unit (a2) is equal to or larger than the lower limit value of the above-described preferred range, the effect obtained by allowing the constitutional unit (a2) to be contained can be sufficiently achieved by the effect described above. In a case where the proportion of the constitutional unit (a2) is equal to or smaller than the upper limit value of the above-described preferred range, balance with other constitutional units can be obtained, and various lithography characteristics are improved.

### In Regard to Constitutional Unit (a8):

The constitutional unit (a8) is a constitutional unit derived from a compound represented by General Formula (a8-1).

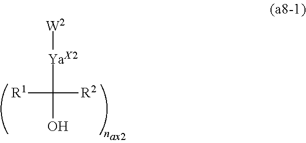

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms, which may have a fluorine atom, or a hydrogen atom. R² and Ya^{x2} may be bonded to each other to form a ring structure. nₐₓ₂ represents an integer in a range of 1 to 3.

The term “polymerizable group” in the polymerizable group-containing group as W² is a group that enables a compound having the polymerizable group to be polymerized by radical polymerization or the like, and refers to a group containing a multiple bonds between carbon atoms, such as an ethylenic double bond.

The “polymerizable group-containing group” may refer to a group composed of only a polymerizable group, or a group composed of a polymerizable group and a group other than the polymerizable group. Examples of the group other than the polymerizable group include a divalent hydrocarbon group which may have a substituent and a divalent linking group containing a hetero atom.

Suitable examples of the polymerizable group-containing group include a group represented by a chemical formula: C(R^{X11})(R^{X12})═C(R^{X13})—Ya^{x10}—.

In this chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}. The condensed ring formed by Ya^{x2} and W² may have a substituent.

### Specific Examples of the Constitutional Unit (a8) are as Follows

In the formulae described below, R^{α} represents a hydrogen atom, a methyl group, or a trifluoromethyl group.

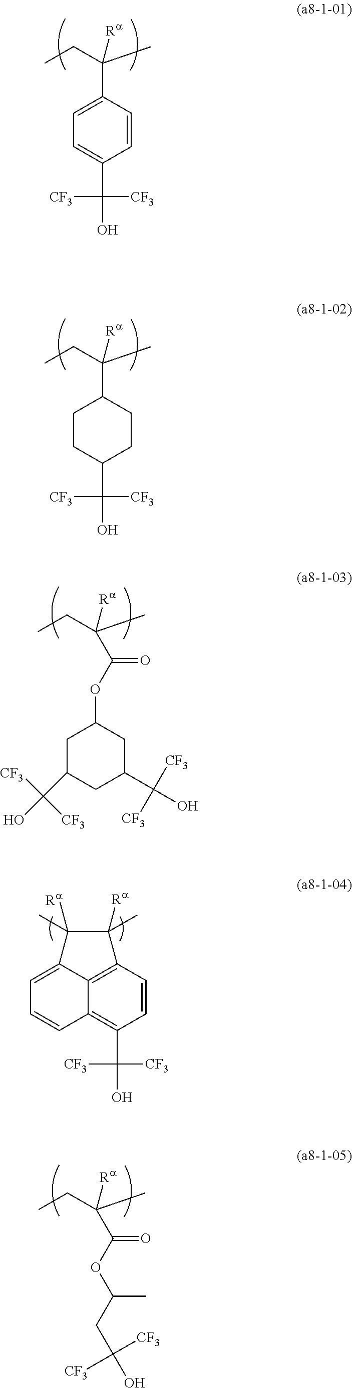

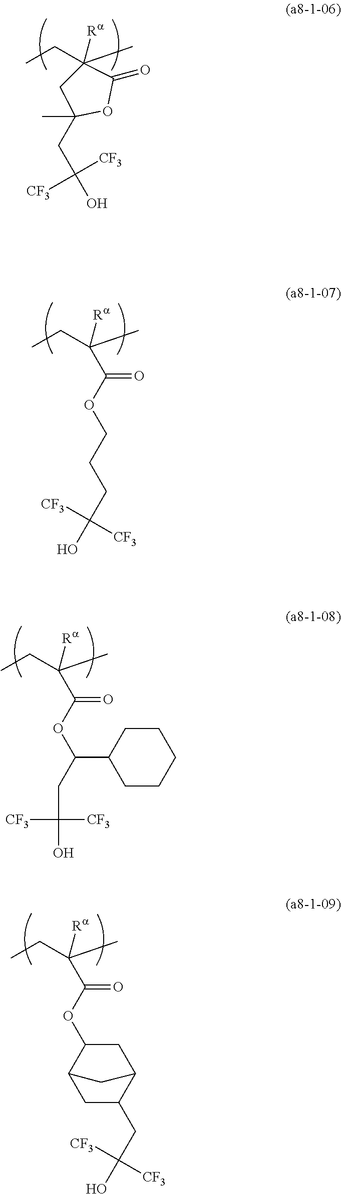

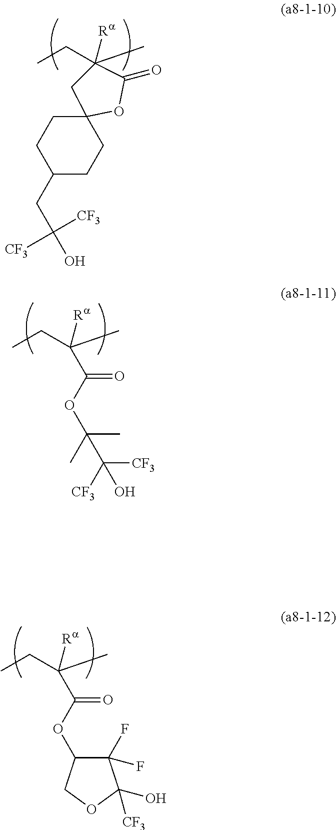

Among the above examples, the constitutional unit (a8) is preferably at least one selected from the group consisting of constitutional units represented by each of Chemical Formulae (a8-1-0l) to (a8-1-04), (a8-1-06), (a8-1-08), (a8-1-09), and (a8-1-10), and more preferably at least one selected from the group consisting of constitutional units represented by each of Chemical Formulae (a8-1-01) to (a8-1-04) and (a8-1-09).

The constitutional unit (a8) contained in the component (A1) may be one kind or may be two or more kinds.

In a case where the component (A1) has the constitutional unit (a8), the proportion of the constitutional unit (a8) is preferably in a range of 10% to 50% by mole, more preferably in a range of 5% to 45% by mole, and still more preferably in a range of 5% to 40% by mole, with respect to the total (100% by mole) of all constitutional units constituting the component (A1).

In a case where the proportion of the constitutional unit (a8) is equal to or larger than the lower limit value of the preferred range, compatibility with the developing solution and the rinse liquid can be enhanced. On the other hand, in a case where the proportion is equal to or smaller than the upper limit value of the preferred range, balance with other constitutional units can be obtained, and various lithography characteristics are improved.

The component (A1) contained in the resist composition may be used alone or in a combination of two or more kinds thereof.

In the resist composition according to the present embodiment, examples of the component (A1) include a polymeric compound having a repeating structure of the constitutional unit (a1), and preferred examples thereof include a polymeric compound having a repeating structure of the constitutional unit (a1) and the constitutional unit (a10).

Among them, suitable examples of the component (A1) include a polymeric compound consisting of a repeating structure of the constitutional unit (a1) and the constitutional unit (a10).

In the polymeric compound having a repeating structure of the constitutional unit (a1) and the constitutional unit (a10), the proportion of the constitutional unit (a1) is preferably in a range of 10% to 90% by mole, more preferably in a range of 20% to 80% by mole, still more preferably in a range of 30% to 70% by mole, and particularly preferably in a range of 40% to 60% by mole with respect to the total (100% by mole) of all constitutional units constituting the polymeric compound.

In addition, the proportion of the constitutional unit (a10) in each of the polymeric compounds described above is preferably in a range of 10% to 90% by mole, more preferably in a range of 20% to 80% by mole, still more preferably in a range of 30% to 70% by mole, and particularly preferably in a range of 40% to 60% by mole, with respect to the total (100% by mole) of all constitutional units constituting the polymeric compound.

The molar ratio of the constitutional unit (a1) to the constitutional unit (a10) in the polymeric compound (constitutional unit (a1):constitutional unit (a2)) is preferably in a range of 2:8 to 8:2, more preferably in a range of 3:7 to 7:3, and still more preferably in a range of 4:6 to 6:4.

The component (A1) can be produced by dissolving, in a polymerization solvent, each monomer from which the constitutional unit is derived, adding thereto a radical polymerization initiator such as azobisisobutyronitrile (AIBN) or dimethyl azobisisobutyrate (for example, V-601) to carry out polymerization.

Alternatively, the component (A1) can be produced by dissolving, in a polymerization solvent, a monomer from which the constitutional unit (a1) is derived, and as necessary, a monomer from which a constitutional unit (for example, the constitutional unit (a10)) other than the constitutional unit (a1) is derived, adding thereto a radical polymerization initiator as described above to carry out polymerization, and then carrying out a deprotection reaction.

It is noted that a —C(CF₃)₂—OH group may be introduced into the terminal during the polymerization by using, in combination, a chain transfer agent such as HS—CH₂—CH₂—CH₂—C(CF₃)₂—OH. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight average molecular weight (Mw) (in terms of the polystyrene equivalent value determined by gel permeation chromatography (GPC)) of the component (A1), which is not particularly limited, is preferably in a range of 1,000 to 50,000, more preferably in a range of 2,000 to 30,000, and still more preferably in a range of 3,000 to 20,000.

In a case where Mw of the component (A1) is equal to or smaller than the upper limit value of this preferred range, the resist composition exhibits sufficient solubility in a resist solvent such that the resist composition can be used as a resist. On the other hand, in a case where Mw thereof is equal to or larger than the lower limit value of this preferred range, dry etching resistance and the cross-sectional shape of the resist pattern become excellent.

Further, the polydispersity (Mw/Mn) of the component (A1) is not particularly limited; however, it is preferably in a range of 1.0 to 4.0, more preferably in a range of 1.0 to 3.0, and particularly preferably in a range of 1.0 to 2.0. Mn represents the number average molecular weight.

### In Regard to Component (A2)

In the resist composition according to the present embodiment, a base material component (hereinafter, referred to as a “component (A2)”) that exhibits changed solubility in a developing solution under the action of an acid, which does not correspond to the component (A1), may be used in combination as the component (A).

The component (A2) is not particularly limited and one freely selected from a large number of base material components for chemical amplification-type resist compositions known in the related art may be used.

As the component (A2), a polymeric compound or a low molecular weight compound may be used alone or in a combination of two or more kinds thereof.

The proportion of the component (A1) in the component (A) is preferably 25% by mass or more, more preferably 50% by mass or more, and still more preferably 75% by mass or more, and it may be 100% by mass with respect to the total mass of the component (A). In a case where the proportion is 25% by mass or more, a resist pattern having various excellent lithography characteristics such as high sensitivity, resolution, and roughness amelioration can be easily formed.

The content of the component (A) in the resist composition according to the present embodiment may be adjusted depending on the resist film thickness to be formed.

### <Acid Diffusion Control Agent Component (D)>

The resist composition according to the present embodiment further contains an acid diffusion control agent component (D) in addition to the component (A).

The component (D) contains a compound (D0) (hereinafter, also referred to as a “component (D0)”) represented by General Formula (d0).

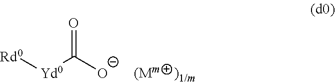

[In the formula, Rd⁰ represents a condensed cyclic group in which an aromatic ring and an alicyclic ring are condensed. The alicyclic ring in the condensed cyclic group has a substituent, where at least one of the substituents includes a hydrocarbon group having a bromine atom or a hydrocarbon group having an iodine atom. Yd⁰ represents a divalent linking group or a single bond. However, Yd⁰ is bonded to the alicyclic ring in the condensed cyclic group. M^{m+} represents an m-valent organic cation. m represents an integer of 1 or more].

### {Anion Moiety of Component (D0)}

In General Formula (d0), Rd⁰ represents a condensed cyclic group in which an aromatic ring and an alicyclic ring are condensed.

The aromatic ring is not particularly limited as long as it is a cyclic conjugated system having 4n+2 π electrons, and the aromatic ring may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 14 carbon atoms.

Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a hetero atom. Examples of the hetero atom in the aromatic heterocyclic rings include an oxygen atom, a sulfur atom, and a nitrogen atom. Specific examples of the aromatic heterocyclic ring include a pyridine ring and a thiophene ring.

The alicyclic ring may be monocyclic or polycyclic. The alicyclic ring preferably has 4 to 30 carbon atoms, more preferably has 4 to 20 carbon atoms, still more preferably has 4 to 15 carbon atoms, and particularly preferably has 4 to 10 carbon atoms.

Specific examples of the alicyclic ring include monocyclic aliphatic rings such as cyclobutane, cyclopentane, cyclohexane, and cyclooctane; polycyclic aliphatic rings such as adamantane, norbornane, isobornane, tricyclodecane, and tetracyclododecane; and an aliphatic heterocyclic ring obtained by substituting some carbon atoms constituting the monocyclic alicyclic ring or the polycyclic alicyclic ring with a hetero atom. Examples of the hetero atom in the aliphatic heterocyclic rings include an oxygen atom, a sulfur atom, and a nitrogen atom. Specific examples of the aliphatic heterocyclic ring include a tetrahydropyran ring, a thian ring, and a piperidine ring.

In the condensed cyclic group as Rd⁰, one aromatic ring may be condensed with one alicyclic ring, two or more aromatic rings may be condensed with one alicyclic ring, two or more alicyclic rings may be condensed with one aromatic ring, or an alicyclic ring and an aromatic ring may be repeatedly condensed. In addition, in a case where a plurality of alicyclic rings and a plurality of aromatic rings are condensed, the plurality of alicyclic rings may be the same or different from each other, and the plurality of aromatic rings may be the same or different from each other.

Among the above, the condensed cyclic group as Rd⁰ is preferably a condensed cyclic group in which one aromatic ring is condensed with one alicyclic ring, or a condensed cyclic group in which two or more aromatic rings are condensed with one alicyclic ring, more preferably a condensed cyclic group in which one aromatic hydrocarbon ring is condensed with one monocyclic aliphatic ring, or a condensed cyclic group in which two or more aromatic hydrocarbon rings are condensed with one monocyclic aliphatic ring, and still more preferably a condensed cyclic group in which two aromatic hydrocarbon rings are condensed with one monocyclic aliphatic ring.

Specific examples of the condensed cyclic group as Rd⁰ include fluorene; and a group in which one or more aromatic rings are condensed with a polycycloalkane having a bridged ring-based polycyclic skeleton. Specific examples of the bridged ring-based polycycloalkane include bicycloalkanes such as bicyclo[2.2.1]heptane (norbornane) and bicyclo[2.2.2]octane.

More specifically, the condensed cyclic group as Rd⁰ is preferably a condensed cyclic group in which two or three aromatic rings are condensed with a bicycloalkane, and more preferably a condensed cyclic group in which two or three aromatic rings are condensed with bicyclo[2.2.2]octane.

Specific examples of the condensed cyclic group as Rd⁰ include groups represented by General Formulae (r-br-1) and (r-br-2). In the formulae, * represents a bonding site for bonding to Yd⁰ in General Formula (d0).

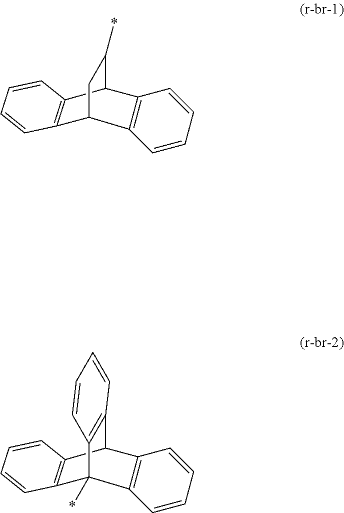

In General Formula (do), the condensed cyclic group as Rd⁰ is, among the above, preferably a condensed cyclic group in which two or three aromatic rings are condensed with a bicycloalkane, more preferably a condensed cyclic group in which two or three aromatic rings are condensed with bicyclo[2.2.2]octane, and still more preferably groups represented by General Formulae (r-br-1) and (r-br-2).

In General Formula (do), the alicyclic ring in the condensed cyclic group as Rd⁰ has a substituent, where at least one of the substituents includes a hydrocarbon group having a bromine atom or a hydrocarbon group having an iodine atom.

Examples of the hydrocarbon group having a bromine atom or the hydrocarbon group in the hydrocarbon group having an iodine atom include a linear or branched alkyl group or a cyclic hydrocarbon group.

The linear alkyl group preferably has 1 to 5 carbon atoms. Specific examples thereof include a methyl group, an ethyl group, an n-propyl group, an n-butyl group, and an n-pentyl group.

The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group.

The cyclic hydrocarbon group may be an aliphatic hydrocarbon group or an aromatic hydrocarbon group, and it may be a polycyclic group or a monocyclic group.

Examples of the cyclic hydrocarbon group include a group obtained by removing one hydrogen atom from the aromatic ring or alicyclic ring in the condensed cyclic group as Rd⁰.

The hydrocarbon group having a bromine atom or the hydrocarbon group in the hydrocarbon group having an iodine atom is, among the above, preferably a cyclic hydrocarbon group and more preferably an aromatic hydrocarbon group.

The hydrocarbon group may have one or more substituents other than the bromine atom and the iodine atom. Examples of the substituent include an alkyl group, a fluorine atom, a chlorine atom, an alkoxy group (a methoxy group, an ethoxy group, a propoxy group, or a butoxy group), a hydroxy group, a cyano group, an amino group, and a nitro group.

In addition, in the hydrocarbon group, some carbon atoms (those in the methylene group or the like) constituting the hydrocarbon group may be substituted with a hetero atom-containing group.

Examples of the hetero atom mentioned here include an oxygen atom, a sulfur atom, and a nitrogen atom. Examples of the hetero atom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂˜O—.

The hydrocarbon group may have a bromine atom and an iodine atom. That is, the alicyclic ring in the condensed cyclic group as Rd⁰ may have a hydrocarbon group having both a bromine atom and an iodine atom.

The total number of bromine atoms and iodine atoms contained in the hydrocarbon group is preferably an integer in a range of 1 to 3, more preferably 2 or 3, and still more preferably 3.

As the total number of bromine atoms and iodine atoms contained in the hydrocarbon group is larger, higher sensitivity tends to be achieved in the resist pattern formation.

Specifically, the substituent contained in the alicyclic ring in the condensed cyclic group as Rd⁰ is preferably a group represented by General Formula (X-1).

*—X⁰¹—Rᵢ⁰¹ (X-1)

[In the formula, X⁰¹ represents a single bond or a divalent linking group. Rᵢ¹⁰ represents a hydrocarbon group having a bromine atom or a hydrocarbon group having an iodine atom. In the formula, * represents a bonding site for bonding to the alicyclic ring in the condensed cyclic group as Rd⁰ in General Formula (d0).]

In General Formula (X-1), X⁰¹ represents a divalent linking group. Suitable examples of the divalent linking group include a divalent linking group containing an oxygen atom.

Examples of divalent linking groups containing an oxygen atom include non-hydrocarbon-based oxygen atom-containing linking groups such as an oxygen atom (an ether bond; —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon-based oxygen atom-containing linking groups with an alkylene group. Further, a sulfonyl group (—SO₂—) may be further linked to the combination.

Examples of the alkylene group include a linear alkylene group and a branched alkylene group.

Examples of the linear alkylene group include a methylene group [—CH₂—], an ethylene group [—(CH₂)₂—], a trimethylene group [—(CH₂)₃—], a tetramethylene group [—(CH₂)₄—], and a pentamethylene group [—(CH₂)₂—].

Examples of the branched alkylene group include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—.

In addition, X⁰1 may be —N(R^{a})—C(═O)—, —N(R^{a})—, —C(R^{a})(R^{a})—N(R^{a})—, —C(R^{a})(N(R^{a})(R^{a}))—, —C(═O)—N(R^{a})—, or a group of a combination of any of these groups and an alkylene group. It is noted that R^{a}'s each independently represent a hydrogen atom or an alkyl group.

In General Formula (X-1), X⁰¹ is preferably —O—, —OCO—, —COO—, or a group of a combination of any of these groups and an alkylene group, more preferably —OCO—, —COO—, or —OCO—, or a group of a combination of —COO— and an alkylene group, and still more preferably —COO—.

It is noted that with regard to the specific examples of X⁰¹, the notation of each linking group matches with the structure in General Formula (X-1). That is, for example, with regard to —COO—, the carbon atom of the alicyclic ring in the condensed cyclic group as Rd⁰ is bonded to the carbon atom in —COO—. In addition, Rᵢ⁰¹ in General Formula (X-1) is bonded to the oxygen atom of —COO—.

In General Formula (X-1), Rᵢ⁰¹ represents a hydrocarbon group having a bromine atom or a hydrocarbon group having an iodine atom, and examples of the hydrocarbon group include the same one as the hydrocarbon group having a bromine atom or the hydrocarbon group in the hydrocarbon group having an iodine atom described above.

In General Formula (X-1), Rᵢ⁰¹ is preferably, among the above, an aromatic hydrocarbon group having a bromine atom or an aromatic hydrocarbon group having an iodine atom, more preferably a phenyl group or naphthyl group having a bromine atom, or a phenyl group or naphthyl group having an iodine atom, and still more preferably a phenyl group having a bromine atom or a phenyl group having an iodine atom.

In General Formula (X-1), Rᵢ⁰¹ may be a hydrocarbon group having both a bromine atom and an iodine atom.

The total number of bromine atoms and iodine atoms contained in the hydrocarbon group is preferably an integer in a range of 1 to 3, more preferably 2 or 3, and still more preferably 3.

As the total number of bromine atoms and iodine atoms contained in the hydrocarbon group is larger, higher sensitivity tends to be achieved in the resist pattern formation.

The hydrocarbon group (the aromatic hydrocarbon group) may have a substituent other than the bromine atom and the iodine atom. In a case where the hydrocarbon group (the aromatic hydrocarbon group) has a substituent other than the bromine atom and the iodine atom, the substituent is preferably an alkyl group having 1 to 5 carbon atoms, a fluorine atom, or a hydroxy group.

In General Formula (d0), Yd⁰ represents a divalent linking group or a single bond. However, Yd⁰ is bonded to the alicyclic ring in the condensed cyclic group.

Suitable examples of the divalent linking group as Yd⁰ include a divalent linking group containing an oxygen atom.

In a case where Yd⁰ represents a divalent linking group containing an oxygen atom, Yd⁰ may contain an atom other than the oxygen atom. Examples of the atom other than the oxygen atom include a carbon atom, a hydrogen atom, a sulfur atom, and a nitrogen atom.

Examples of the divalent linking group containing an oxygen atom include the same one as the above-described divalent linking group containing an oxygen atom, as X⁰¹.

Yd⁰ is preferably a divalent linking group containing an ester bond or a divalent linking group containing an ether bond, and more preferably a linking group represented by General Formulae (y-d0-1) or (y-d0-2).

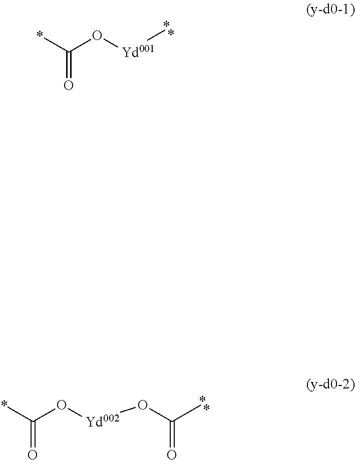

[In the formulae, Yd⁰⁰¹ and Yd⁰⁰² each independently represent a hydrocarbon group having 1 to 6 carbon atoms, which may have a substituent. * represents a bonding site for bonding to the alicyclic ring in the condensed cyclic group as Rd⁰ in General Formula (d0). ** represents a bonding site to a carbon atom of the carbonyl group in General Formula (do).]

Yd^{0 01} in General Formula (y-d0-1) and Yd⁰⁰² in General Formula (y-d0-2) each independently represent a hydrocarbon group having 1 to 6 carbon atoms, which may have a substituent.

The hydrocarbon group may be an aromatic hydrocarbon group or an aliphatic hydrocarbon.

In addition, the hydrocarbon group may have a substituent. Examples of the substituent include a halogen atom, a hydroxy group, a cyano group, an amino group, and a nitro group.

Specific examples of the aromatic hydrocarbon group include a phenylene group.

Examples of the aliphatic hydrocarbon group include an alkylene group, an alkenylene group, an alkadienylene group, an alkatrienylene group, an alkynylene group, or a group of a combination of these groups.

### Alkylene Group Having 1 to 4 Carbon Atoms

Examples of the linear alkylene group having 1 to 4 carbon atoms include a methylene group, an ethylene group [—(CH₂)₂—], a trimethylene group [—(CH₂)₃—], and a tetramethylene group [—(CH₂)₄—].

Examples of the branched alkylene group having 2 to 4 carbon atoms include alkylalkylene groups such as an alkylmethylene group such as —CH(CH₃)—, —CH(CH₂CH₃)—, —C(CH₃)₂—, or —C(CH₃)(CH₂CH₃)—; an alkylethylene group such as —CH(CH₃)CH₂—, —CH(CH₃)CH(CH₃)—, —C(CH₃)₂CH₂—, or —CH(CH₂CH₃)CH₂—; and an alkyltrimethylene group such as —CH(CH₃)CH₂CH₂— or CH₂CH(CH₃)CH₂—.

### Alkenylene Group Having 2 to 4 Carbon Atoms

The alkenylene group having 2 to 4 carbon atoms may be a linear alkenylene group or may be a branched alkenylene group.

Examples of the linear alkenylene group having 2 to 4 carbon atoms include an ethenylene group (a vinylene group), a 1-propenylene group, a 2-propenylene group, and a butynylene group.

Examples of the branched alkenylene group having 3 or 4 carbon atoms include a 1-methylvinylene group, a 1-methylpropenylene group, and a 2-methylpropenylene group.

### Alkadienylene Group and Alkatrienylene Group

Examples of the alkadienylene group having 3 or 4 carbon atoms include a propadienylene group and a butadienylene group, and examples of the alkatrienylene group having 4 carbon atoms include a butatrienylene group.

### Alkynylene Group Having 2 to 4 Carbon Atoms

Examples of the alkynylene group having 2 to 4 carbon atoms include an ethynylene group (—C≡C—).

The group of the combination of an alkylene group, an alkenylene group, an alkadienylene group, an alkatrienylene group, and an alkynylene group is preferably, for example, a group of a combination of an alkylene group and an alkynylene group. Specifically, a —CH₂—C≡C—group is preferable.

Among the above, Yd⁰⁰¹ in General Formula (y-d0-1) is preferably a phenylene group which may have a substituent, an alkylene group having 1 to 4 carbon atoms, or a group of a combination of an alkylene group and an alkynylene group, the group having 1 to 4 carbon atoms in total.

In a case where the phenylene group which may have a substituent has a substituent, the substituent is preferably a halogen atom and more preferably a fluorine atom.

Among the above, Yd⁰⁰² in General Formula (y-d0-1) is preferably an alkylene group having 1 to 4 carbon atoms.

In the present embodiment, the anion moiety of the component (D0) is preferably an anion represented by General Formula (d0-an0) from the viewpoint of improvement of sensitivity and CDU.

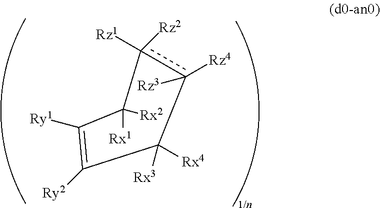

[In the formula, Rx¹ to Rx⁴ each independently represent a hydrocarbon group which may have a substituent or a hydrogen atom, or two or more of Rx¹ to Rx⁴ may be bonded to each other to form a ring structure. Ry¹ and Ry² each independently represent a hydrocarbon group which may have a substituent or a hydrogen atom, or may be bonded to each other to form a ring structure.

[symbol]

The above double line of the dotted line and the straight line represents a double bond or a single bond. Where valence permits, Rz¹ to Rz⁴ each independently represent a hydrocarbon group which may have a substituent or a hydrogen atom, or two or more of Rz¹ to Rz⁴ may be bonded to each other to form a ring structure. However, two or more of Rx¹ to Rx⁴, Ry¹ and Ry², or two or more of Rz¹ to Rz⁴ are bonded to each other to form an aromatic ring. In addition, at least one among Rx¹ to Rx⁴, Ry¹ and Ry², and Rz¹ to Rz⁴ has an anion group represented by General Formula (d0-r-an1), where the entire anion moiety is an n-valent anion. In addition, at least one of Rx¹ to Rx⁴, Ry¹ and Ry², and Rz¹ to Rz⁴ includes a hydrocarbon group having a bromine atom or a hydrocarbon group having an iodine atom. n represents an integer of 1 or more.]

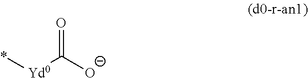

[In the formula, Yd⁰ represents a divalent linking group or a single bond. * represents a bonding site.]

In General Formula (d0-an0), Rx^{I} to Rx⁴ each independently represent a hydrocarbon group which may have a substituent or a hydrogen atom, or two or more of Rx¹ to Rx⁴ may be bonded to each other to form a ring structure.

Ry¹ and Ry² each independently represent a hydrocarbon group which may have a substituent or a hydrogen atom, or may be bonded to each other to form a ring structure.

Where valence permits, Rz^{I} to Rz⁴ each independently represent a hydrocarbon group which may have a substituent or a hydrogen atom, or two or more of Rz¹ to Rz⁴ may be bonded to each other to form a ring structure.

The hydrocarbon group as Rx¹ to Rx⁴, Ry¹, Ry², and Rz¹ to Rz⁴ may each be an aliphatic hydrocarbon group or an aromatic hydrocarbon group and may each be a cyclic hydrocarbon group or a chain-like hydrocarbon group.

Examples of the hydrocarbon group which may have a substituent, as Rx¹ to Rx⁴, Ry¹, Ry², and Rz¹ to Rz⁴, include a cyclic group which may have a substituent, a chain-like alkyl group which may have a substituent, and a chain-like alkenyl group which may have a substituent.

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group that has no aromaticity. The aliphatic hydrocarbon group may be saturated or unsaturated. In general, it is preferable that the aliphatic hydrocarbon group be saturated. In addition, the cyclic hydrocarbon group as Rx¹ to Rx⁴, Ry¹, Ry², and Rz¹ to Rz⁴, may have a hetero atom such as a heterocyclic ring.

The aromatic hydrocarbon group as Rx1 to Rx4, Ry1, Ry2, and Rz1 to Rz4 is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. However, the number of carbon atoms in a substituent is not included in the number of carbon atoms.

Specific examples of the aromatic ring contained in the aromatic hydrocarbon group as Rx¹ to Rx⁴, Ry¹, Ry², and Rz¹ to Rz⁴ include benzene, fluorene, naphthalene, anthracene, phenanthrene, biphenyl, and an aromatic heterocyclic ring obtained by substituting some carbon atoms constituting any one of these aromatic rings with a hetero atom. Examples of the hetero atom in the aromatic heterocyclic rings include an oxygen atom, a sulfur atom, and a nitrogen atom. The aromatic ring contained in the aromatic hydrocarbon group as Rx¹ to Rx⁴, Ry¹, Ry², and Rz¹ to Rz⁴ preferably does not contain a hetero atom and is more preferably an aromatic ring such as benzene, fluorene, naphthalene, anthracene, phenanthrene, or biphenyl from the viewpoint of compatibility with the component (A).

Specific examples of the aromatic hydrocarbon group as Rx¹ to Rx⁴, Ry¹, Ry², and Rz¹ to Rz⁴ include a group obtained by removing one hydrogen atom from the above-described aromatic ring (an aryl group such as a phenyl group or a naphthyl group) and a group in which one hydrogen atom in the aromatic ring has been substituted with an alkylene group (an arylalkyl group such as a benzyl group, a phenethyl group, a 1-naphthylmethyl group, a 2-naphthylmethyl group, 1-naphthylethyl group, or a 2-naphthylethyl group). The alkylene group (an alkyl chain in the arylalkyl group) preferably has 1 to 4 carbon atoms, more preferably has 1 or 2 carbon atoms, and particularly preferably has 1 carbon atom.

Examples of the cyclic aliphatic hydrocarbon group as Rx¹ to Rx⁴, Ry¹, Ry², and Rz¹ to Rz⁴ include an aliphatic hydrocarbon group containing a ring in the structure thereof.

Examples of the aliphatic hydrocarbon group containing a ring in the structure thereof include an alicyclic hydrocarbon group (a group obtained by removing one hydrogen atom from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane preferably has 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms.

The linear aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and most preferably has 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group preferably has 2 to 10 carbon atoms, more preferably has 3 to 6 carbon atoms, still more preferably has 3 or 4 carbon atoms, and most preferably has 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, examples of the cyclic group as Rx¹ to Rx⁴, Ry¹, Ry², and Rz¹ to Rz⁴ include —COOR^{XYZ} and —OC(═O)R^{XYZ} % where R^{XYZ} is a lactone-containing cyclic group or —SO₂—containing cyclic group.

The term “—SO₂—containing cyclic group” denotes a cyclic group having a ring containing —SO₂— in the ring skeleton thereof. Specifically, the —SO₂—containing cyclic group is a cyclic group in which the sulfur atom (S) in —SO₂— forms a part of the ring skeleton of the cyclic group. In a case where the ring containing —SO₂—in the ring skeleton thereof is counted as the first ring and the group has only that ring, the group is referred to as a monocyclic group. In a case where the group further has other ring structures, the group is referred to as a polycyclic group regardless of the structures. The —SO₂—containing cyclic group may be a monocyclic group or a polycyclic group. The —SO₂—containing cyclic group is particularly preferably a cyclic group containing —O—SO₂— in the ring skeleton thereof, that is, a cyclic group containing a sultone ring in which —O—S— in —O—SO₂— forms a part of the ring skeleton thereof. More specific examples of the —SO₂—containing cyclic group include groups represented by each of General Formulae (b5-r-1) to (b5-r-4) shown below.

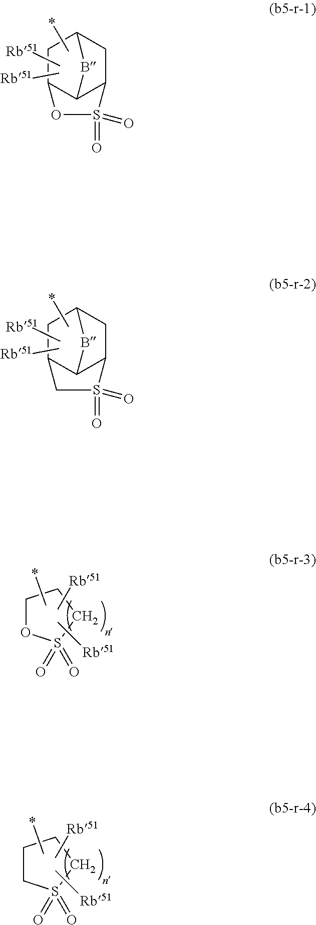

[In the formulae, each Rb′⁵¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or an —SO₂—containing cyclic group; B″ represents an oxygen atom, a sulfur atom, or an alkylene group having 1 to 5 carbon atoms, which may contain an oxygen atom or a sulfur atom; and n′ represents an integer in a range of 0 to 2. * represents a bonding site.]

In General Formulae (b5-r-1) and (b5-r-2), B″ represents an alkylene group having 1 to 5 carbon atoms which may contain an oxygen atom or a sulfur atom, an oxygen atom, or a sulfur atom.

B″ is preferably an alkylene group having 1 to 5 carbon atoms or —O—, more preferably an alkylene group having 1 to 5 carbon atoms, and still more preferably a methylene group.

In General Formulae (b5-r-1) to (b5-r-4), each Rb′⁵¹ independently represents a hydrogen atom, an alkyl group, an alkoxy group, a halogen atom, a halogenated alkyl group, a hydroxyl group, —COOR″, —OC(═O)R″, a hydroxyalkyl group, or a cyano group, and among the above, each Rb′⁵¹ is independently preferably a hydrogen atom or a cyano group.

Specific examples of the groups represented by each of General Formulae (b5-r-1) to (b5-r-4) are shown below. In the formulae shown below, “Ac” represents an acetyl group.

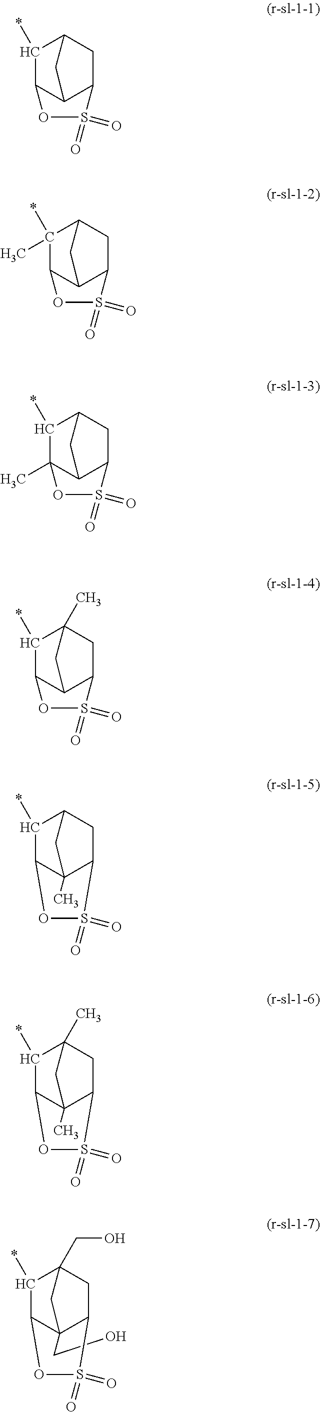

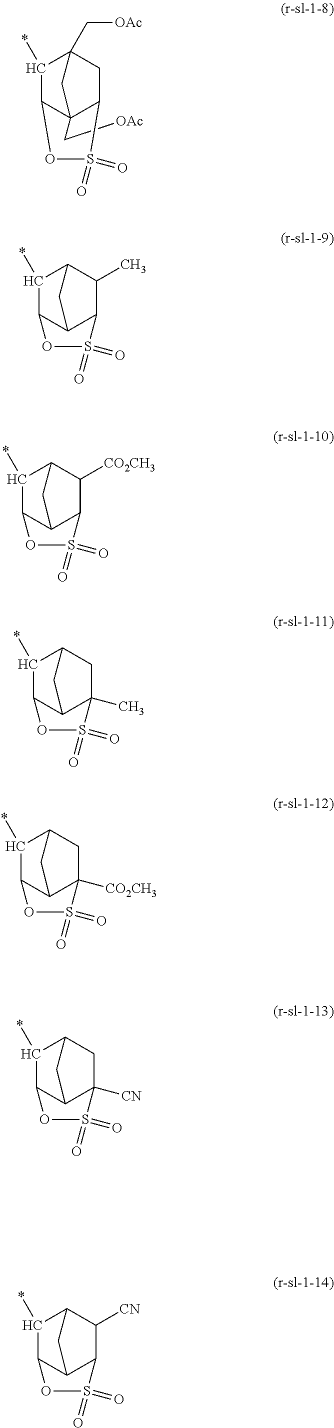

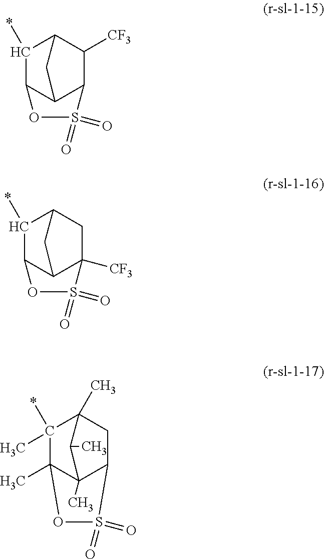

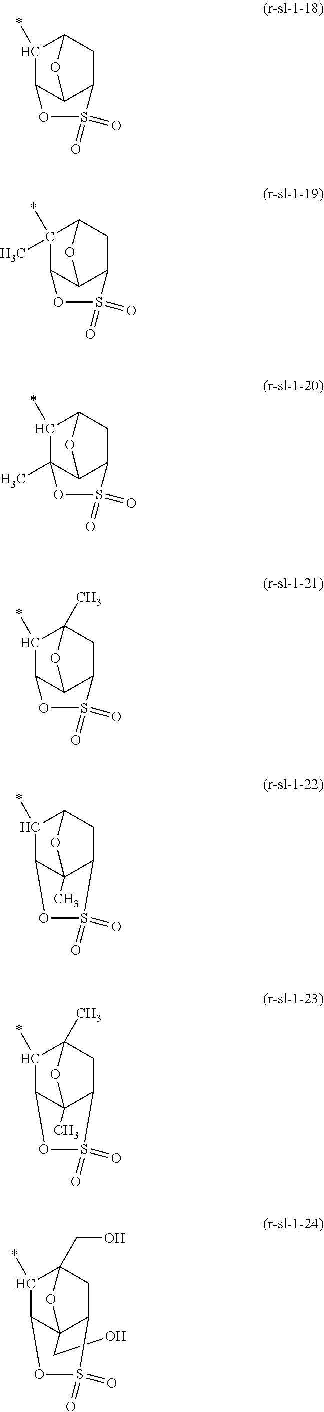

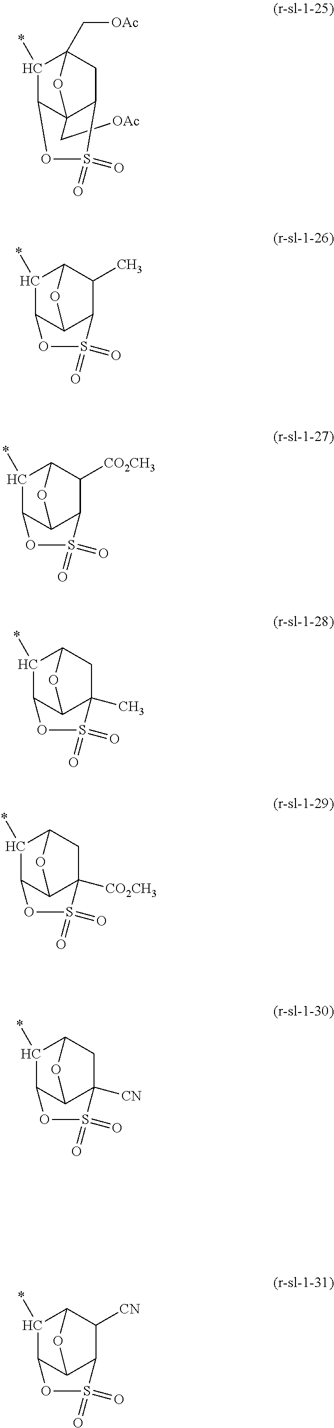

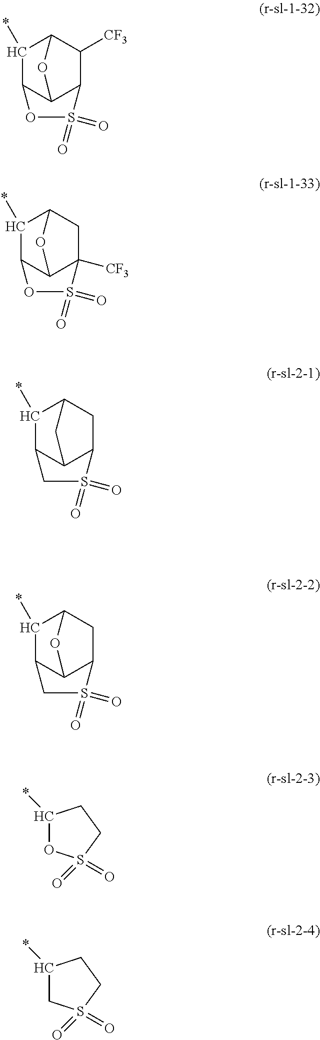

Examples of the substituent in the cyclic group as Rx¹ to Rx⁴, Ry¹, Ry², and Rz¹ to Rz⁴ include the same substituent as the substituent which may be contained in the polycyclic aromatic cyclic group as Rd⁰.

Among the above, the substituent in the cyclic group as Rx¹ to Rx⁴, Ry¹, Ry², Rz¹ to Rz⁴ is preferably an alkyl group, a halogen atom, or a halogenated alkyl group from the viewpoint of compatibility with the component (A).

Chain-like alkyl group which may have substituent: The chain-like alkyl group as Rx¹ to Rx⁴, Ry¹, Ry², and Rz¹ to Rz⁴ may be either linear or branched.

The linear alkyl group preferably has 1 to 20 carbon atoms, more preferably has 1 to 15 carbon atoms, and most preferably has 1 to 10 carbon atoms. Specific examples thereof include a methyl group, an ethyl group, a propyl group, a butyl group, a pentyl group, a hexyl group, a heptyl group, an octyl group, a nonyl group, a decanyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a henicosyl group, and a docosyl group.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1,1-dimethylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent: The chain-like alkenyl group as Rx¹ to Rx⁴, Ry¹, Ry², and Rz¹ to Rz⁴ may be linear or branched, and it preferably has 2 to 10 carbon atoms, more preferably has 2 to 5 carbon atoms, still more preferably has 2 to 4 carbon atoms, and particularly preferably has 3 carbon atoms. Examples of the linear alkenyl group include a vinyl group, a propenyl group (an allyl group), and a butynyl group. Examples of the branched alkenyl group include a 1-propenyl group, a 2-propenyl group (an allyl group), a 1-methylpropenyl group, and a 2-methylpropenyl group.

Examples of the substituent in the chain-like alkyl group or alkenyl group as Rx¹ to Rx⁴, Ry¹, Ry², and Rz¹ to Rz⁴ include an alkoxy group, a halogen atom, a halogenated alkyl group, a hydroxyl group, a carbonyl group, a nitro group, an amino group, and the cyclic group as Rx¹ to Rx⁴, Ry¹, Ry², and Rz¹ to Rz⁴. Among them, the substituent in the chain-like alkyl group or alkenyl group as Rx¹ to Rx⁴, Ry¹, Ry², and Rz¹ to Rz⁴ is preferably a halogen atom, a halogenated alkyl group, or the group exemplified as the cyclic group as Rx¹ to Rx⁴, Ry¹, Ry², and Rz¹ to Rz⁴ from the viewpoint of compatibility with the component (A).

In General Formula (d0-an0), Ry¹ and Ry² may be bonded to each other to form a ring structure.

The ring structure formed by Ry¹ and Ry² shares one side (the bond between carbon atoms to which Ry¹ and Ry² are each bonded) of the 6-membered ring in General Formula (d0-an0), and this ring structure may be an alicyclic hydrocarbon or may be an aromatic hydrocarbon. Further, this ring structure may be a polycyclic structure consisting of other ring structures.

The alicyclic hydrocarbons formed by R¹ and Ry² may be polycyclic or monocyclic. The monocyclic alicyclic hydrocarbon is preferably a monocycloalkane. The monocycloalkane preferably has 3 to 6 carbon atoms, and specific examples thereof include cyclopentane and cyclohexane. The polycyclic alicyclic hydrocarbon is preferably a polycycloalkane. The polycycloalkane preferably has 7 to 30 carbon atoms.

Specific examples of the aromatic hydrocarbon ring that are formed by Ry¹ and Ry² include benzene, fluorene, naphthalene, anthracene, phenanthrene, biphenyl, or an aromatic heterocyclic ring obtained by substituting some carbon atoms constituting these aromatic rings with a hetero atom. The aromatic hydrocarbon ring formed by Ry¹ and Ry² preferably does not contain a hetero atom and is more preferably an aromatic ring such as benzene, fluorene, naphthalene, anthracene, phenanthrene, or biphenyl from the viewpoint of compatibility with the component (A).

The ring structure (the alicyclic hydrocarbon or the aromatic hydrocarbon) formed by Ry¹ and Ry² may have a substituent. Examples of the substituent here include the same ones as the substituents (for example, an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among them, the substituent in the ring structure formed by Ry¹ and Ry² is preferably an alkyl group, a halogen atom, or a halogenated alkyl group from the viewpoint of compatibility with the component (A).

Among the above, the ring structure formed by Ry¹ and Ry² is more preferably an aromatic hydrocarbon which may have a substituent.

In General Formula (d0-an0), two or more of Rz¹ to Rz⁴ may be bonded to each other to form a ring structure. For example, Rz¹ may form a ring structure together with any one of Rz² to Rz⁴. Specific examples thereof include a ring structure that shares one side (the bond between a carbon atom to which Rz^{I} and Rz² are bonded and a carbon atom to which Rz³ and Rz⁴ are bonded) of the 6-membered ring in General Formula (d0-an0), a ring structure formed by bonding Rz¹ and Rz², and a ring structure formed by bonding Rz³ and Rz⁴.

The ring structure formed by two or more of Rz¹ to Rz⁴ may be an alicyclic hydrocarbon or an aromatic hydrocarbon and is preferably an aromatic hydrocarbon. Further, this ring structure may be a polycyclic structure consisting of other ring structures.

The alicyclic hydrocarbon formed by two or more of Rz¹ to Rz⁴ may be polycyclic or monocyclic. The monocyclic alicyclic hydrocarbon is preferably a monocycloalkane. The monocycloalkane preferably has 3 to 6 carbon atoms, and specific examples thereof include cyclopentane and cyclohexane. The polycyclic alicyclic hydrocarbon is preferably a polycycloalkane. The polycycloalkane is preferably a polycycloalkane having 7 to 30 carbon atoms, and specifically, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

It may be a heterocyclic structure obtained by substituting some carbon atoms with a hetero atom and particularly preferably a nitrogen-containing heterocyclic ring, and specific examples thereof include a cyclic imide.

Specific examples of the aromatic hydrocarbon ring that is formed by two or more of Rz^{I} to Rz⁴ include benzene, fluorene, naphthalene, anthracene, phenanthrene, biphenyl, or an aromatic heterocyclic ring obtained by substituting some carbon atoms constituting these aromatic rings with a hetero atom. The aromatic hydrocarbon ring formed by two or more of Rz¹ to Rz⁴ preferably does not contain a hetero atom, and is more preferably an aromatic ring such as benzene, fluorene, naphthalene, anthracene, phenanthrene, or biphenyl from the viewpoint of compatibility with the component (A).

The ring structure (the alicyclic hydrocarbon or the aromatic hydrocarbon) formed by Rz¹ to Rz⁴ may have a substituent. Examples of the substituent here include the same ones as the substituents (for example, an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among them, the substituent in the ring structure formed by Rz¹ to Rz⁴ is preferably an alkyl group, a halogen atom, or a halogenated alkyl group from the viewpoint of compatibility with the component (A).

Among the above, the ring structure formed by two or more of Rz¹ to Rz⁴ is preferably a ring structure that shares one side (the bond between a carbon atom to which Rz¹ and Rz² are bonded and a carbon atom to which Rz³ and Rz⁴ are bonded) of the 6-membered ring in General Formula (d0-an0) and more preferably an aromatic ring structure.

In General Formula (d0-an0), “where valence permits” has the following meaning.

That is, in a case where the bond between a carbon atom to which Rz¹ and Rz² are bonded and a carbon atom to which Rz³ and Rz⁴ are bonded is a single bond, all of Rz¹, Rz², Rz³, and Rz⁴ are present. In a case where the bond between the carbon atom to which Rz¹ and Rz² are bonded and the carbon atom to which Rz³ and Rz⁴ are bonded is a double bond, only one of Rz¹ and Rz² is present, and only one of Rz³ and Rz⁴ is present. In addition, for example, in a case where Rz¹ and Rz³ are bonded to form an aromatic ring structure, Rz² and Rz⁴ are not present.

In General Formula (d0-an0), two or more of Rx¹ to Rx⁴ may be bonded to each other to form a ring structure. For example, Rx¹ may form a ring structure together with any one of Rx² to Rx⁴.

The ring structure formed by two or more of Rx¹ to Rx⁴ may be an alicyclic hydrocarbon or an aromatic hydrocarbon. Further, this ring structure may be a polycyclic structure consisting of other ring structures.

The alicyclic hydrocarbon formed by two or more of Rx¹ to Rx⁴ may be polycyclic or monocyclic. The monocyclic alicyclic hydrocarbon is preferably a monocycloalkane. The monocycloalkane preferably has 3 to 6 carbon atoms, and specific examples thereof include cyclopentane and cyclohexane. The polycyclic alicyclic hydrocarbon is preferably a polycycloalkane. The polycycloalkane is preferably a polycycloalkane having 7 to 30 carbon atoms, and specifically, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

Specific examples of the aromatic hydrocarbon ring that is formed by two of Rx¹ to Rx⁴ include benzene, fluorene, naphthalene, anthracene, phenanthrene, biphenyl, or an aromatic heterocyclic ring obtained by substituting some carbon atoms constituting these aromatic rings with a hetero atom. The aromatic hydrocarbon ring formed by two of Rx¹ to Rx⁴ preferably does not contain a hetero atom, and is more preferably an aromatic ring such as benzene, fluorene, naphthalene, anthracene, phenanthrene, or biphenyl from the viewpoint of compatibility with the component (A).

The ring structure (the alicyclic hydrocarbon or the aromatic hydrocarbon) formed by Rx¹ to Rx⁴ may have a substituent. Examples of the substituent here include the same ones as the substituents (for example, an alkyl group, an alkoxy group, a halogen atom, a halogenated alkyl group, a hydroxyl group, a nitro group, and a carbonyl group) in the above-described cyclic group as Rx¹ to Rx⁴, Ry¹, Ry², and Rz¹ to Rz⁴. Among them, the substituent in the ring structure formed by Rx¹ to Rx⁴ is preferably an alkyl group, a halogen atom, or a halogenated alkyl group from the viewpoint of compatibility with the component (A).

The ring structure formed by two or more of Rx¹ to Rx⁴ is preferably an alicyclic hydrocarbon.

Further, among the above, it is preferable that the ring structure formed by two or more of Rx¹ to Rx⁴ be a ring structure in which at least one of Rx¹ and Rx² and at least one of Rx³ to Rx⁴ are bonded to each other to form a bridged ring structure, and it is more preferable that this ring structure be an alicyclic hydrocarbon.

In a case where at least one of Rx¹ and Rx² and at least one of Rx³ and Rx⁴ are bonded to each other to form a ring structure, the number of carbon atoms constituting the bicyclic structure (the ring structure which also contains carbon atoms each bonded to Ry¹, Ry², Rz¹ and Rz², and Rz³ and Rz⁴) is preferably 7 to 16.

In General Formula (d0-an0), two or more of Rx¹ to Rx⁴, Ry¹ and Ry², or two or more of Rz¹ to Rz⁴ are bonded to each other to form an aromatic ring. The aromatic ring is the same as the aromatic ring described in the explanation of General Formula (do).

In General Formula (d0-an0), at least one among Rx¹ to Rx⁴, Ry¹ and Ry², and Rz¹ to Rz⁴ has an anion group represented by General Formula (d0-r-an1), where the entire anion moiety is an n-valent anion. n represents an integer of 1 or more. Rx¹ to Rx⁴, Ry¹, Ry², and Rz¹ to Rz⁴ may each be the above-described anion group. In a case where two or more of Rx¹ to Rx⁴ are bonded to each other to form a ring structure, a carbon atom that forms the ring structure or a hydrogen atom bonded to this carbon atom may be substituted with the above anion group. In a case where two or more of Ry¹ and Ry² are bonded to each other to form a ring structure, a carbon atom that forms the ring structure or a hydrogen atom bonded to this carbon atom may be substituted with the above anion group. In a case where two or more of Rz¹ to Rz⁴ are bonded to each other to form a ring structure, a carbon atom that forms the ring structure or a hydrogen atom bonded to this carbon atom may be substituted with the above anion group.

In General Formula (d0-r-an1), the divalent linking group as Yd⁰ is the same as the divalent linking group as Yd⁰ in General Formula (d0).

The number of anion groups in the component (D0) may be one or two or more.

In the component (D0), the entire anion moiety is an n-valent anion. n represents an integer of 1 or more, preferably represents 1 or 2, and more preferably 1.

In General Formula (d0-an0), at least one among Rx¹ to Rx⁴, Ry¹ and Ry², and Rz¹ to Rz⁴ includes a hydrocarbon group having a bromine atom or a hydrocarbon group having an iodine atom. The preferred aspect of the hydrocarbon group having a bromine atom or the hydrocarbon group having an iodine atom is the same as the hydrocarbon group having a bromine atom or the hydrocarbon group having an iodine atom described in General Formula (d0).

The anion moiety in the component (D0) is more preferably an anion represented by General Formula (d0-an1) from the viewpoint of improvement of sensitivity and CDU.

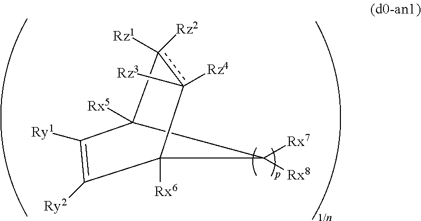

[In the formula, Rx⁵ and Rx⁶ each independently represent a hydrocarbon group which may have a substituent or a hydrogen atom. Rx⁷ and Rx⁸ each independently represent a hydrocarbon group which may have a substituent or a hydrogen atom, or may be bonded to each other to form a ring structure. p is 1 or 2, and in a case where p=2, a plurality of Rx⁷'s and Rx⁸'s may be different from each other. Ry¹ and Ry² each independently represent a hydrocarbon group which may have a substituent or a hydrogen atom, or may be bonded to each other to form a ring structure.

[symbol]

The above double line of the dotted line and the straight line represents a double bond or a single bond. Where valence permits, Rz¹ to Rz⁴ each independently represent a hydrocarbon group which may have a substituent or a hydrogen atom, or two or more of Rz¹ to Rz⁴ may be bonded to each other to form a ring structure. However, Rx⁵ and Rx⁶, Rx⁷ and Rx⁸, Ry¹ and Ry², or two or more of Rz¹ to Rz⁴ are bonded to each other to form an aromatic ring. In addition, at least one among Rx⁵ to Rx⁸, Ry¹ and Ry², and Rz¹ to Rz⁴ has an anion group represented by General Formula (d0-r-an1), where the entire anion moiety is an n-valent anion. In addition, at least one among Rx⁵ to Rx⁸ Ry¹ and Ry², and Rz¹ to Rz⁴ includes a hydrocarbon group having a bromine atom or a hydrocarbon group having an iodine atom. n represents an integer of 1 or more.]

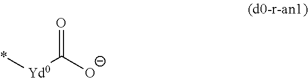

[In the formula, Yd⁰ represents a divalent linking group or a single bond. * represents a bonding site.]

In General Formula (d0-an1), Rx⁵ and Rx⁶ each independently represent a hydrocarbon group which may have a substituent or a hydrogen atom. The hydrocarbon group which may have a substituent, as Rx⁸ and Rx⁶, is the same as that described in the explanation of the hydrocarbon group which may have a substituent as Rx¹ to Rx⁴ in General Formula (d0-an0).

In General Formula (d0-an1), Rx⁷ and Rx⁸ each independently represent a hydrocarbon group which may have a substituent or a hydrogen atom or may be bonded to each other to form a ring structure. Rx⁷ and Rx⁸ are the same as those described in the explanation of Rx¹ to Rx⁴ in General Formula (d0-an0).

In General Formula (d0-an1), p is 1 or 2, and in a case where p=2, a plurality of Rx⁷'s and Rx⁸'s may be different from each other. In the case of p=1, the anion represented by General Formula (d0-an1) has a bicycloheptane ring structure, and in the case of p=2, the anion has a bicyclooctane ring structure.

In General Formula (d0-an1), Ry¹ and Ry² each independently represent a hydrocarbon group which may have a substituent or a hydrogen atom or may be bonded to each other to form a ring structure. Ry¹ and Ry² are the same as Ry¹ and Ry² in General Formula (d0-an0).

Where valence permits, Rz¹ to Rz⁴ each independently represent a hydrocarbon group which may have a substituent or a hydrogen atom, or two or more of Rz¹ to Rz⁴ may be bonded to each other to form a ring structure. Rz¹ to Rz⁴ are the same as Rz¹ to Rz⁴ in General Formula (d0-an0).

In General Formula (d0-an1), Rx^{S} and Rx⁶, Rx⁷ and Rx⁸, Ry¹ and Ry², or two or more of Rz¹ to Rz⁴ are bonded to each other to form an aromatic ring. The details of the aromatic ring are as described in the explanation in General Formula (d0).

In General Formula (d0-an1), at least one among Rx⁵ to Rx⁸, Ry¹ and Ry², and Rz¹ to Rz⁴ has an anion group represented by General Formula (d0-r-an1), where the entire anion moiety is an n-valent anion. n represents an integer of 1 or more, preferably represents 1 or 2, and more preferably 1.

In General Formula (d0-an1), at least one among Rx⁸ to Rx⁸, Ry¹ and Ry², and Rz¹ to Rz⁴ includes a hydrocarbon group having a bromine atom or a hydrocarbon group having an iodine atom. The preferred aspect of the hydrocarbon group having a bromine atom or the hydrocarbon group having an iodine atom is the same as the hydrocarbon group having a bromine atom or the hydrocarbon group having an iodine atom described in General Formula (d0).

Among the above, the anion moiety in the component (D0) is still more preferably an anion represented by General Formula (d0-an1) in the case of p=2, that is, an anion represented by General Formula (d0-an2).

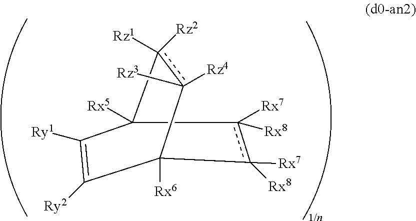

[In the formula, Rx⁸ and Rx⁶ each independently represent a hydrocarbon group which may have a substituent or a hydrogen atom. A plurality of Rx⁷ and Rx⁸ each independently represent a hydrocarbon group which may have a substituent or a hydrogen atom, or two or more Rx⁷ and Rx⁸ may be bonded to each other to form a ring structure. Ry¹ and Ry² each independently represent a hydrocarbon group which may have a substituent or a hydrogen atom, or may be bonded to each other to form a ring structure.

[symbol]

The above double line of the dotted line and the straight line represents a double bond or a single bond. Where valence permits, Rz¹ to Rz⁴ each independently represent a hydrocarbon group which may have a substituent or a hydrogen atom, or two or more of Rz¹ to Rz⁴ may be bonded to each other to form a ring structure. However, Rx⁵ and Rx⁶, two or more of Rx⁷ and Rx⁸, Ry¹ and Ry², or two or more of Rz¹ to Rz⁴ are bonded to each other to form an aromatic ring. In addition, at least one among Rx⁵ to Rx⁸, Ry¹ and Ry², and Rz¹ to Rz⁴ has an anion group represented by General Formula (d0-r-an1), where the entire anion moiety is an n-valent anion. In addition, at least one among Rx⁵ to Rx⁸, Ry¹ and Ry², and Rz¹ to Rz⁴ includes a hydrocarbon group having a bromine atom or a hydrocarbon group having an iodine atom. n represents an integer of 1 or more.]

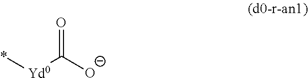

[In the formula, Yd⁰ represents a divalent linking group or a single bond. * represents a bonding site.]

In General Formula (d0-an2), Rx⁵ and Rx^{b}, Rx⁷ and Rx⁸, Ry¹ and Ry², and Rz¹ to Rz⁴ are the same as Rx⁵ and Rx⁶, Rx⁷ and Rx⁸, Ry¹ and Ry², and Rz¹ to Rz⁴ in General Formula (d0-an1), respectively.

In General Formula (d0-an2), Rx⁵ and Rx⁶, two or more of Rx⁷ and Rx⁸, Ry¹ and Ry², or two or more of Rz¹ to Rz⁴ are bonded to each other to form an aromatic ring. The details of the aromatic ring are as described in the explanation in General Formula (do).

In General Formula (d0-an2), at least one among Rx⁵ to Rx⁸, Ry¹ and Ry², and Rz¹ to Rz⁴ has an anion group represented by General Formula (d0-r-an1), where the entire anion moiety is an n-valent anion. n represents an integer of 1 or more, preferably represents 1 or 2, and more preferably 1.

In General Formula (d0-an2), at least one among Rx⁵ to Rx⁸, Ry¹ and Ry², and Rz¹ to Rz⁴ includes the hydrocarbon group having a bromine atom or the hydrocarbon group having an iodine atom described above. The preferred aspect of the hydrocarbon group having a bromine atom or the hydrocarbon group having an iodine atom is the same as the hydrocarbon group having a bromine atom or the hydrocarbon group having an iodine atom described in General Formula (do).

In General Formula (d0-an0), General Formula (d0-an1), and General Formula (d0-an2) described above, it is preferable that Ry¹ and Ry² be bonded to each other to form a ring structure, and it is more preferable that the ring structure to be formed be an aromatic hydrocarbon (an aromatic ring or an aromatic heterocyclic ring) which may have a substituent.

In General Formula (d0-an0), General Formula (d0-an1), and General Formula (d0-an2) described above, it is preferable that Rz¹ to Rz⁴ be bonded to each other to form a ring structure, where the ring structure to be formed is preferably a ring structure that shares one side (the bond between a carbon atom to which Rz¹ and Rz² are bonded and a carbon atom to which Rz³ and Rz⁴ are bonded) of the 6-membered ring in the formula and more preferably an aromatic hydrocarbon (an aromatic ring or an aromatic heterocyclic ring) which may have a substituent.

In General Formulae (d0-an1) and (d0-an2) described above, it is preferable that Rx⁷ and Rx⁸ be bonded to each other to form a ring structure, and it is more preferable that the ring structure to be formed is an aromatic hydrocarbon (an aromatic ring or an aromatic heterocyclic ring) which may have a substituent.

In General Formula (d0-an2), the ring structure formed in Rx⁷ and Rx⁸ is preferably a ring structure that shares one side (the bond between the same carbon atoms to which Rx⁷ and Rx⁸ are bonded) of the 6-membered ring in the formula and more preferably an aromatic hydrocarbon (an aromatic ring or an aromatic heterocyclic ring) which may have a substituent.

In the entire anion represented by General Formula (d0-an2) described above, the number of ring structures each formed by bonding Rx⁷ and Rx⁸, Ry¹ and Ry², and Rz¹ to Rz⁴ to each other may be 1 or may be 2 or more, and it is preferably 2 or 3.

In the present embodiment, the anion moiety of the component (D0) is particularly preferably an anion represented by General Formula (d0-an3) from the viewpoint of improvement of sensitivity and CDU.

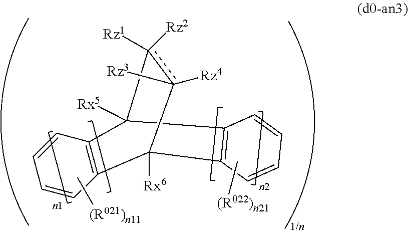

[In the formula, Rx⁵ and Rx⁶ each independently represent a hydrocarbon group which may have a substituent or a hydrogen atom.

[symbol]

The above double line of the dotted line and the straight line represents a double bond or a single bond. Where valence permits, Rz¹ to Rz⁴ each independently represent a hydrocarbon group which may have a substituent or a hydrogen atom, or two or more of Rz¹ to Rz⁴ may be bonded to each other to form a ring structure. However, at least one among Rx⁵ and Rx⁶ and Rz¹ to Rz⁴ has an anion group represented by General Formula (d0-r-an1), where the entire anion moiety is an n-valent anion. n represents an integer of 1 or more. In addition, at least one among Rx⁵ and Rx⁶ and Rz¹ to Rz⁴ includes a hydrocarbon group having a bromine atom or a hydrocarbon group having an iodine atom. R⁰²¹ represents an alkyl group, an alkoxy group, a halogen atom, a halogenated alkyl group, a hydroxyl group, a carbonyl group, or a nitro group. n1 represents an integer in a range of 1 to 3. n11 represents an integer in a range of 0 to 8. R⁰²² represents an alkyl group, an alkoxy group, a halogen atom, a halogenated alkyl group, a hydroxyl group, a carbonyl group, or a nitro group. n2 represents an integer in a range of 1 to 3. n21 represents an integer in a range of 0 to 8.]

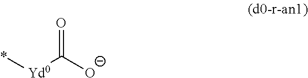

[In the formula, Yd⁰ represents a divalent linking group or a single bond. * represents a bonding site.]

In General Formula (d0-an3), Rx⁵ and Rx⁶ and Rz¹ to Rz⁴ are the same as Rx⁵ and Rx⁶ and Rz¹ to Rz⁴ in General Formula (d0-an1), respectively.

In General Formula (d0-an3), R⁰²¹ represents an alkyl group, an alkoxy group, a halogen atom, a halogenated alkyl group, a hydroxy group, a carbonyl group, or a nitro group.

The alkyl group as R⁰²¹ is preferably an alkyl group having 1 to 5 carbon atoms, and a methyl group, an ethyl group, a propyl group, an n-butyl group, or a tert-butyl group is more preferable.

The alkoxy group as R⁰²¹ is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as R⁰²¹ is preferably a fluorine atom.

Examples of the halogenated alkyl group as R⁰²¹ include a group in which some or all hydrogen atoms in an alkyl group having 1 to 5 carbon atoms such as a methyl group, an ethyl group, a propyl group, an n-butyl group, or a tert-butyl group have been substituted with the above-described halogen atom.

Among them, R⁰²¹ is preferably an alkyl group, a halogen atom, or a halogenated alkyl group from the viewpoint of compatibility with the component (A).

In General Formula (d0-an3), n1 represents an integer in a range of 1 to 3, preferably 1 or 2, and more preferably 1.

In General Formula (d0-an3), n11 represents an integer in a range of 0 to 8, preferably an integer in a range of 0 to 4, more preferably 0, 1, or 2, and still more preferably 0 or 1.

In General Formula (d0-an3), R⁰²² represents an alkyl group, an alkoxy group, a halogen atom, a halogenated alkyl group, a hydroxy group, a carbonyl group, or a nitro group, and examples thereof include those of R⁰² described above. Among them, R⁰²² is preferably an alkyl group, a halogen atom, or a halogenated alkyl group from the viewpoint of compatibility with the component (A).

In General Formula (d0-an3), n2 represents an integer in a range of 1 to 3, preferably 1 or 2, and particularly preferably 1.

In General Formula (d0-an3), n21 represents an integer in a range of 0 to 8, preferably an integer in a range of 0 to 4, more preferably 0, 1, or 2, and particularly preferably 0 or 1.

However, in General Formula (d0-an3), at least one among Rx⁵ and Rx⁶ and Rz¹ to Rz⁴ has an anion group represented by General Formula (d0-r-an1), where the entire anion moiety is an n-valent anion. n represents an integer of 1 or more, preferably represents 1 or 2, and more preferably 1.

In General Formula (d0-an3), at least one among Rx⁵ and Rx⁶ and Rz¹ to Rz⁴ includes the hydrocarbon group having a bromine atom or the hydrocarbon group having an iodine atom described above. The preferred aspect of the hydrocarbon group having a bromine atom or the hydrocarbon group having an iodine atom is the same as the hydrocarbon group having a bromine atom or the hydrocarbon group having an iodine atom described in General Formula (d0).

In General Formula (d0-an0), General Formula (d0-an1), General Formula (d0-an2), and General Formula (d0-an3) described above, at least one of Rz¹ to Rz⁴ preferably has an anion group from the viewpoint of the excellent effect of the present invention. In a case where two or more of Rz¹ to Rz⁴ are bonded to each other to form a ring structure, a carbon atom that forms the ring structure or a hydrogen atom bonded to this carbon atom may be substituted with the above anion group.

In General Formula (d0-an0), General Formula (d0-an1), General Formula (d0-an2), and General Formula (d0-an3) described above, from the viewpoint of the excellent effect of the present invention, at least one of Rz¹ to Rz⁴ preferably includes the hydrocarbon group having a bromine atom or the hydrocarbon group having an iodine atom described above. In a case where two or more of Rz¹ to Rz⁴ are bonded to each other to form a ring structure, a hydrogen atom bonded to a carbon atom that forms the ring structure may be substituted with the hydrocarbon group having a bromine atom or the hydrocarbon group having an iodine atom.

Specific examples of the anion moiety of the component (D0) are shown below.

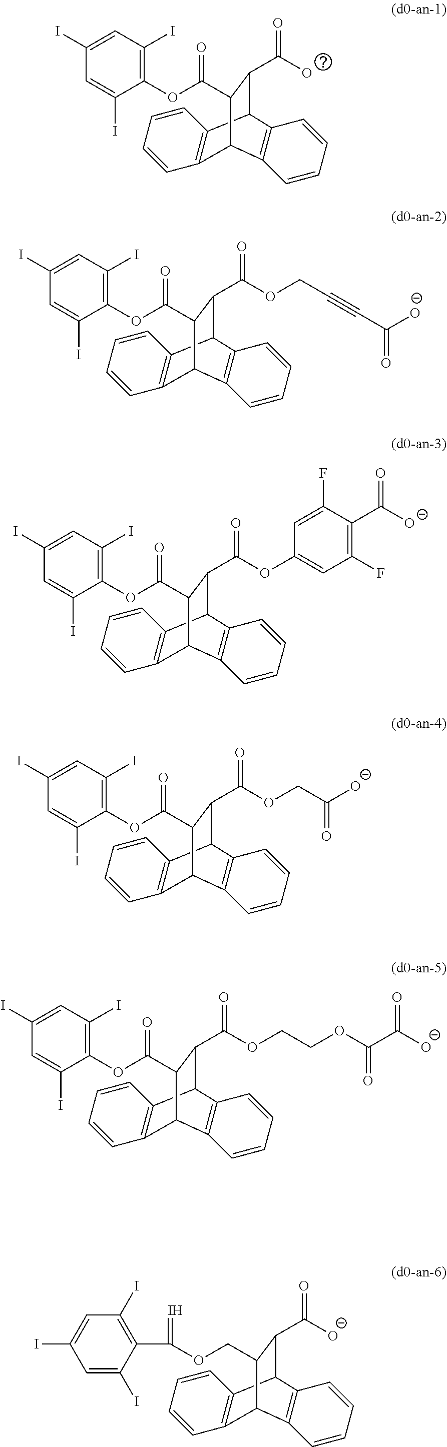

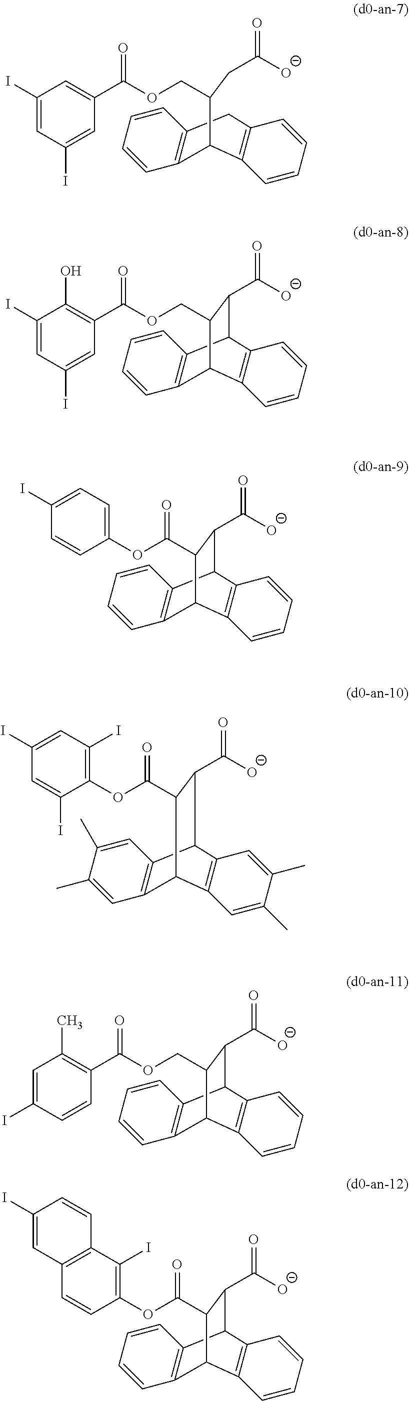 [SMILES: O=C([O-])CC(COC(=O)c1cc(I)cc(I)c1)C1c2ccccc2Cc2ccccc21 ; O=C(OCC1C2c3ccccc3C(c3ccccc32)C1C(=O)[O-])c1cc(I)cc(I)c1O ; O=C([O-])C1C2c3ccccc3C(c3ccccc32)C1C(=O)Oc1ccc(I)cc1 ; Cc1cc2c(cc1C)C1c3cc(C)c(C)cc3C2C(C(=O)[O-])C1C(=O)Oc1c(I)cc(I)cc1I ; Cc1cc(I)ccc1C(=O)OCC1C2c3ccccc3C(c3ccccc32)C1C(=O)[O-] ; O=C([O-])C1C2c3ccccc3C(c3ccccc32)C1C(=O)Oc1ccc2cc(I)ccc2c1I]

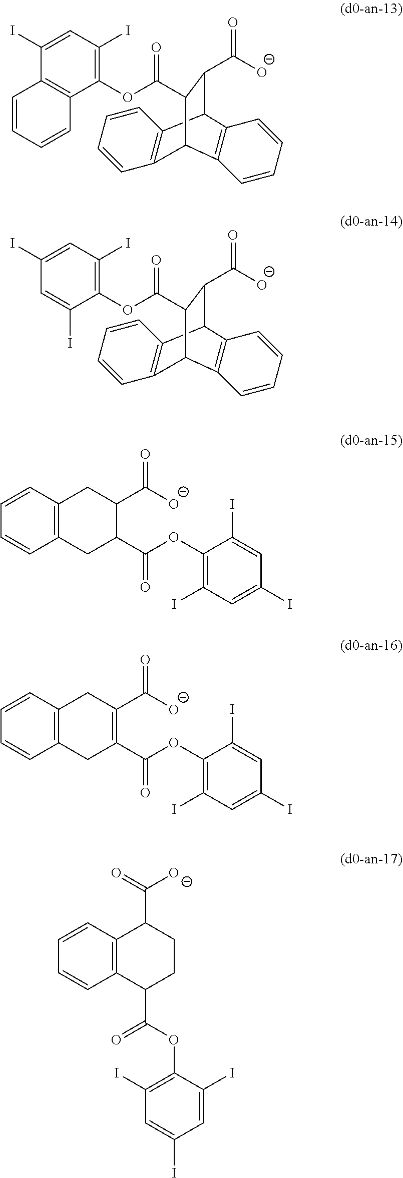 [SMILES: O=C([O-])C1C2c3ccccc3C(c3ccccc32)C1C(=O)Oc1c(I)cc(I)c2ccccc12 ; O=C([O-])C1C2c3ccccc3C(c3ccccc32)C1C(=O)Oc1c(I)cc(I)cc1I ; O=C([O-])C1Cc2ccccc2CC1C(=O)Oc1c(I)cc(I)cc1I ; O=C([O-])C1=C(C(=O)Oc2c(I)cc(I)cc2I)Cc2ccccc2C1 ; O=C([O-])C1CCC(C(=O)Oc2c(I)cc(I)cc2I)c2ccccc21]

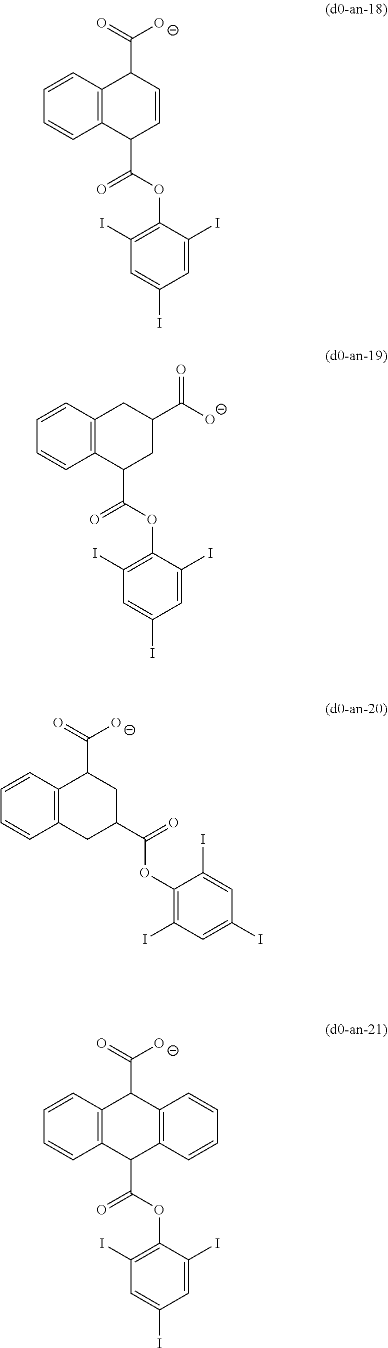 [SMILES: O=C([O-])C1C=CC(C(=O)Oc2c(I)cc(I)cc2I)c2ccccc21 ; O=C([O-])C1Cc2ccccc2C(C(=O)Oc2c(I)cc(I)cc2I)C1 ; O=C(Oc1c(I)cc(I)cc1I)C1Cc2ccccc2C(C(=O)[O-])C1 ; O=C([O-])C1c2ccccc2C(C(=O)Oc2c(I)cc(I)cc2I)c2ccccc21]

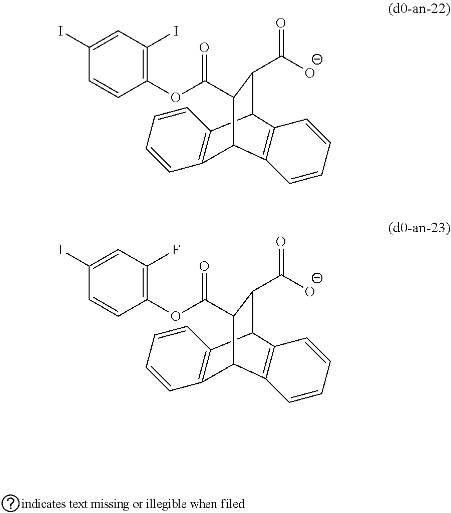 [SMILES: O=C([O-])C1C2c3ccccc3C(c3ccccc32)C1C(=O)Oc1ccc(I)cc1I ; O=C([O-])C1C2c3ccccc3C(c3ccccc32)C1C(=O)Oc1ccc(I)cc1F]

Among the above, the anion moiety of the component (D0) is preferably an anion represented by any one of Chemical Formulae (d0-an-1) to (d0-an-14), (d0-an-22), and (d0-an-23), more preferably an anion represented by any one of Chemical Formulae (d0-an-1) to (d0-an-9), (d0-an-22), and (d0-an-23), still more preferably an anion represented by any one of Chemical Formulae (d0-an-1) to (d0-an-5), and particularly preferably an anion represented by Chemical Formula (d0-an-1) or (d0-an-5).

### {Cation Moiety of Component (D0)}

In General Formula (d0), M^{m+} represents an m-valent organic cation. Among them. a sulfonium cation and an iodonium cation are preferable.

m represents an integer of 1 or more.

Examples of the preferred cation moiety ((M^{m+})_{1/m}) include an organic cation represented by each of General Formulae (ca-1) to (ca-3) described below.

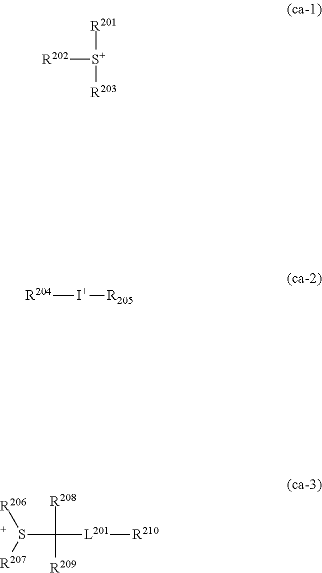

[In the formulae, R²⁰¹ to R²⁰⁷ each independently represent an aryl group, an alkyl group, or an alkenyl group, each of which may have a substituent. R²⁰¹ to R²⁰³, and R²⁰⁶ and R²⁰⁷ may be bonded to each other to form a ring together with the sulfur atom in the formula. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or an —SO₂—containing cyclic group which may have a substituent. L²⁰¹ represents —C(═O)— or —C(═O)—O—.]

In General Formulae (ca-1) to (ca-3), examples of the aryl group as R²⁰ to R²⁰⁷ include an unsubstituted aryl group having 6 to 20 carbon atoms, and a phenyl group or a naphthyl group is preferable.

The alkyl group as R²¹1 to R²⁰⁷ is a chain-like or cyclic alkyl group, and preferably has 1 to 30 carbon atoms.

It is preferable that the alkenyl group as R²⁰¹ to R²⁰⁷ have 2 to 10 carbon atoms.

Examples of the substituent which may be contained in R²⁰¹ to R²⁰⁷ and R²¹⁰ include an alkyl group, a halogen atom, a halogenated alkyl group, a carbonyl group, a cyano group, an amino group, an aryl group, and groups represented by each of General Formulae (ca-r-1) to (ca-r-7) described below.

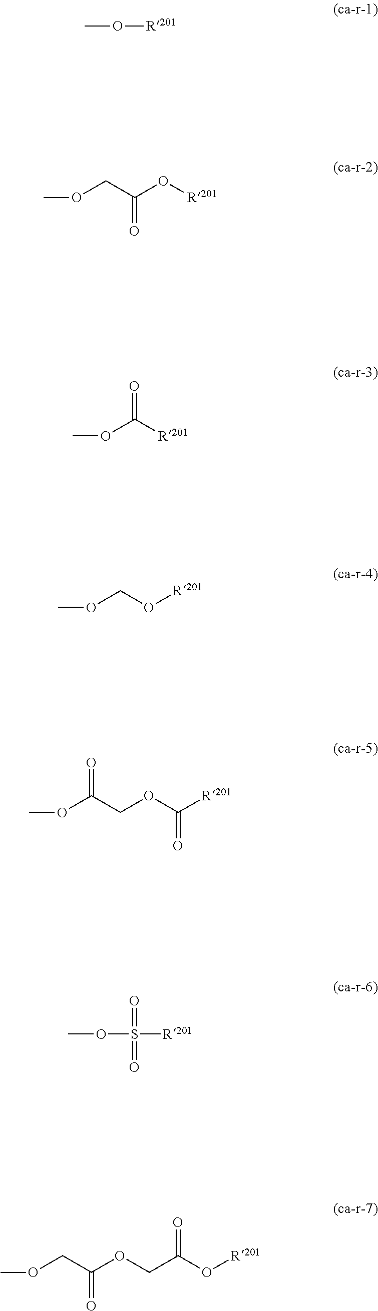

[In the formulae, R′²⁰¹'s each independently represent a hydrogen atom, a cyclic group which may have a substituent, a chain-like alkyl group which may have a substituent, or a chain-like alkenyl group which may have a substituent.]

Cyclic group which may have substituent: The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group that has no aromaticity. The aliphatic hydrocarbon group may be saturated or unsaturated. In general, it is preferable that the aliphatic hydrocarbon group be saturated.

The aromatic hydrocarbon group as R′²⁰ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R′²¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The alkylene group (an alkyl chain in the arylalkyl group) preferably has 1 to 4 carbon atoms, more preferably has 1 or 2 carbon atoms, and particularly preferably has 1 carbon atom.

Examples of the cyclic aliphatic hydrocarbon group as R′²⁰ include an aliphatic hydrocarbon group having a ring in the structure thereof.

Examples of the aliphatic hydrocarbon group containing a ring in the structure thereof include an alicyclic hydrocarbon group (a group obtained by removing one hydrogen atom from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane preferably has 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among the above, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and particularly preferably has 1 to 3 carbon atoms.

As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₂—].

As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Further, the cyclic hydrocarbon group as R′²⁰¹ may have a hetero atom such as a heterocyclic ring. Specific examples thereof include the lactone-containing cyclic groups represented by each of General Formulae (a2-r-1) to (a2-r-7), the —SO₂—containing cyclic groups represented by each of General Formulae (b5-r-1) to (b5-r-4), and other heterocyclic groups represented by each of Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as the substituent is preferably a fluorine atom.

Examples of the above-described halogenated alkyl group as the substituent include a group in which part or all of hydrogen atoms in an alkyl group having 1 to 5 carbon atoms such as a methyl group, an ethyl group, a propyl group, an n-butyl group, or a tert-butyl group have been substituted with the above-described halogen atom.

The carbonyl group as the substituent is a group that substitutes a methylene group (—CH₂—) constituting the cyclic hydrocarbon group.

Chain-like alkyl group which may have substituent: The chain-like alkyl group as R′²⁰ may be linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a I-methylethyl group, a 1-methylpropyl group, a 2-methylpropyl group, a 1-methylbutyl group, a 2-methylbutyl group, a 3-methylbutyl group, a I-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent: The chain-like alkenyl group as R201 may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a 1-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹

Examples of the cyclic group which may have a substituent, the chain-like alkyl group which may have a substituent, and the chain-like alkenyl group which may have a substituent as R′²⁰¹ include those for the acid dissociable group represented by General Formula (a1-r-2) which are the exemplary examples of the cyclic group which may have a substituent and the chain-like alkyl group which may have a substituent, in addition to those described above.

Among the examples, R′²⁰¹ is preferably a cyclic group which may have a substituent and more preferably a cyclic hydrocarbon group which may have a substituent. More specific examples thereof preferably include a phenyl group, a naphthyl group, a group obtained by removing one or more hydrogen atoms from a polycycloalkane, lactone-containing cyclic groups represented by any one of General Formulae (a2-r-1) to (a2-r-7), and —SO₂—containing cyclic groups represented by any one of General Formulae (b5-r-1) to (b5-r-4).

In General Formulae (ca-1) to (ca-3) described above, in a case where R²⁰¹ to R²⁰³ or R²⁰⁶ and R²⁰⁷ are bonded to each other to form a ring together with the sulfur atom in the formula, these groups may be bonded to each other through a hetero atom such as a sulfur atom, an oxygen atom, or a nitrogen atom, or a functional group such as a carbonyl group, —SO—, —SO₂—, —SO₃—, —COO—, —CONH—, or —N(RN)— (here, RN represents an alkyl group having 1 to 5 carbon atoms). Regarding the ring to be formed, it is preferable that a ring containing the sulfur atom in the formula in the ring skeleton thereof be a 3-membered to 10-membered ring and it is particularly preferable that it be a 5-membered to 7-membered ring in a case where the sulfur atom is included. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and are preferably a hydrogen atom or an alkyl group having 1 to 3 carbon atoms. In a case where R²⁰⁸ and R²⁰⁹ represent alkyl groups, R²⁰⁸ and R²⁰¹ may be bonded to each other to form a ring.

R²¹⁰ represents an aryl group which may have a substituent, an alkyl group which may have a substituent, an alkenyl group which may have a substituent, or an —SO₂—containing cyclic group which may have a substituent.

Examples of the aryl group as R²¹⁰ include an unsubstituted aryl group having 6 to 20 carbon atoms. Among these, a phenyl group or a naphthyl group is preferable.

The alkyl group as R²¹⁰ is preferably a chain-like or cyclic alkyl group, and preferably has 1 to 30 carbon atoms.

It is preferable that the alkenyl group as R²¹⁰ have 2 to 10 carbon atoms. The —SO₂—containing cyclic group which may have a substituent, as R²¹⁰, is preferably an “—SO₂—containing polycyclic group”, and more preferably a group represented by General Formula (b5-r-1).

Specific examples of the suitable cation represented by General Formula (ca-1) include cations represented by each of Chemical Formulae (ca-1-1) to (ca-1-113) described below.

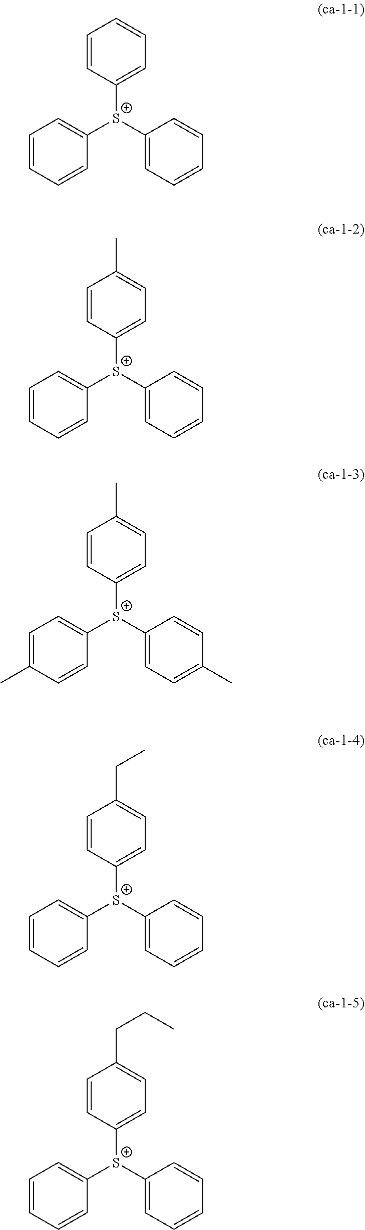

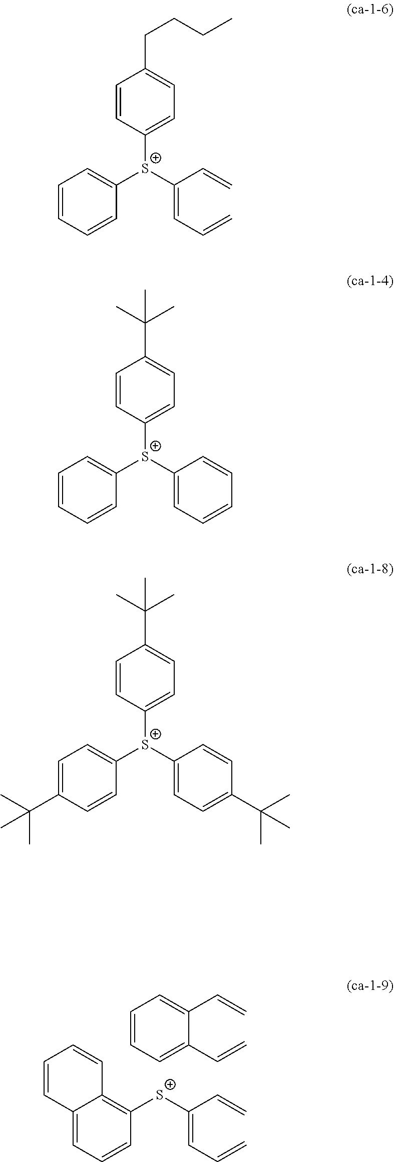

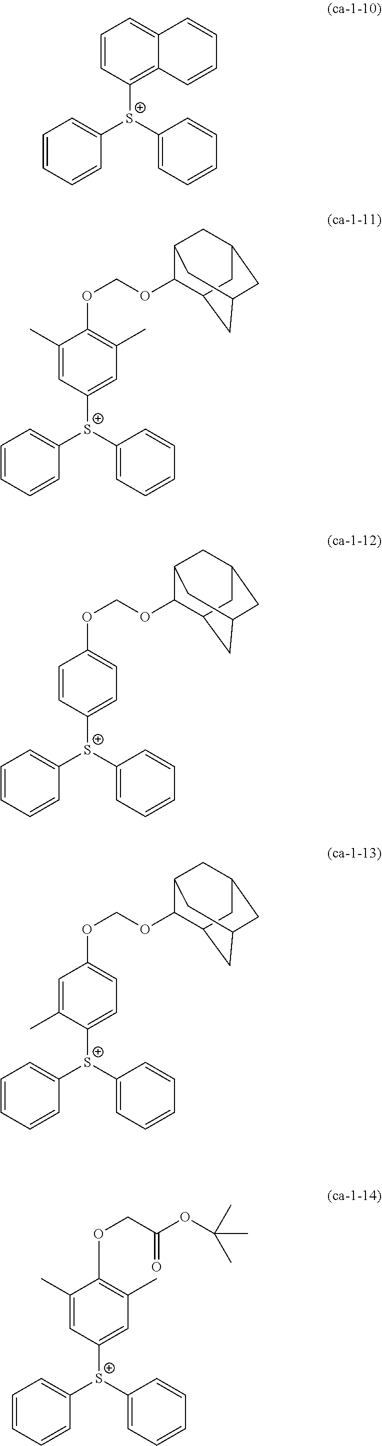

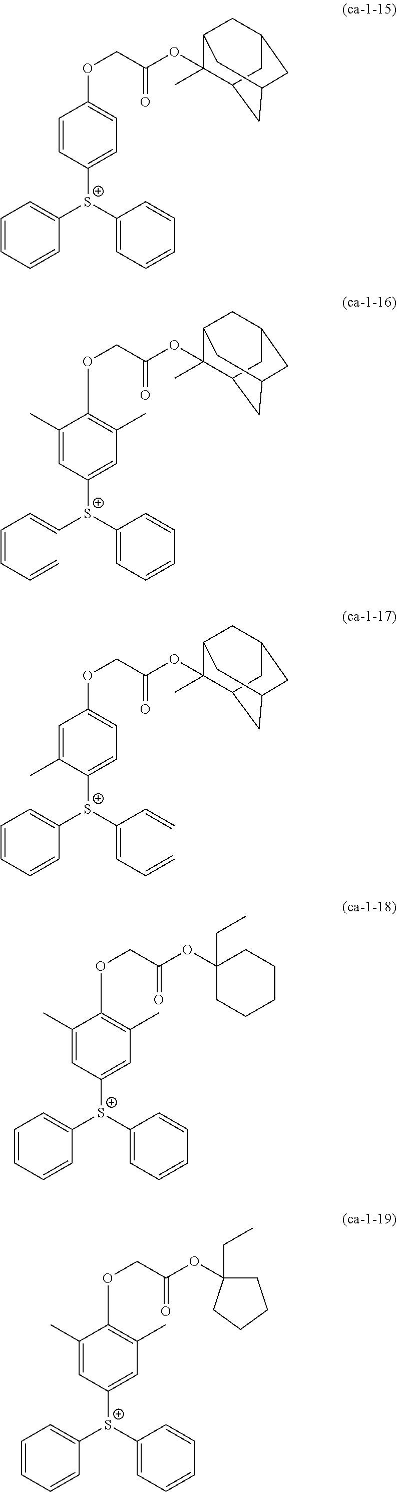

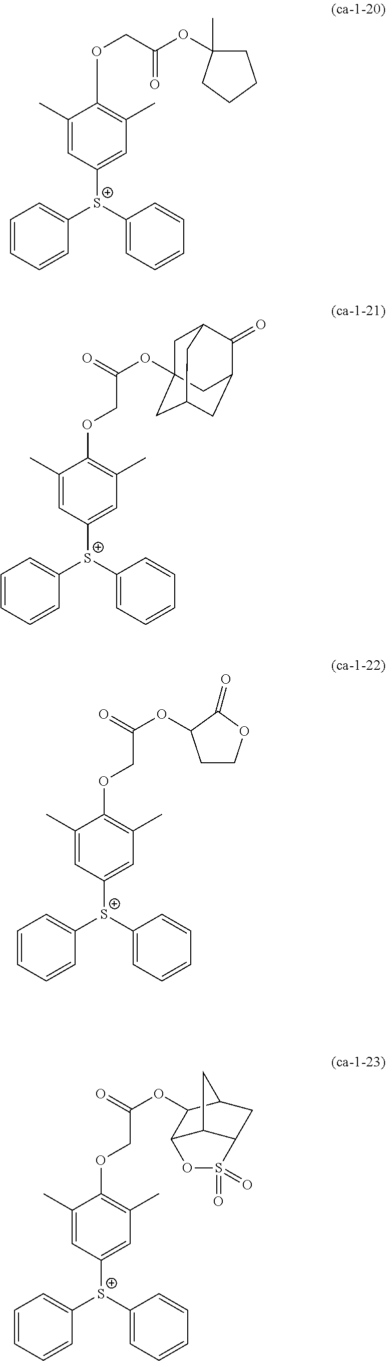

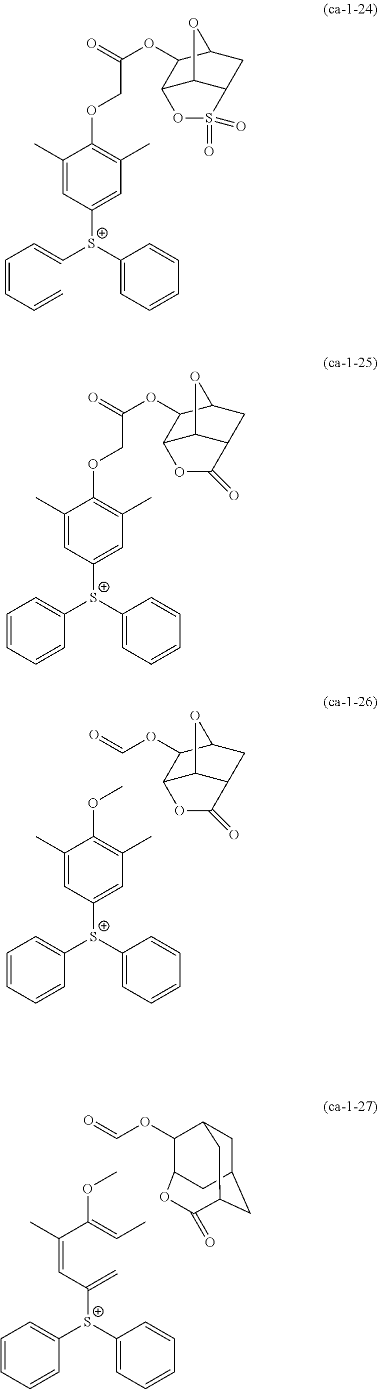

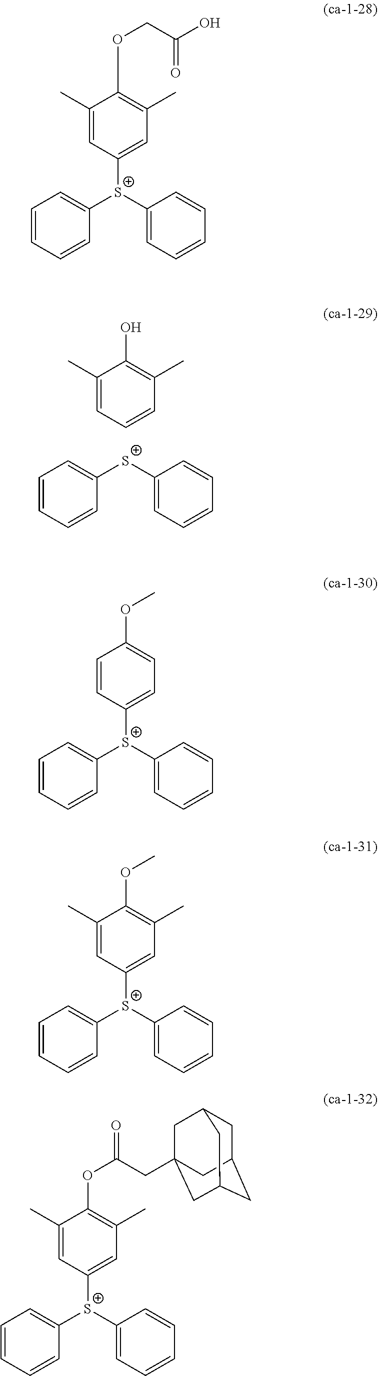

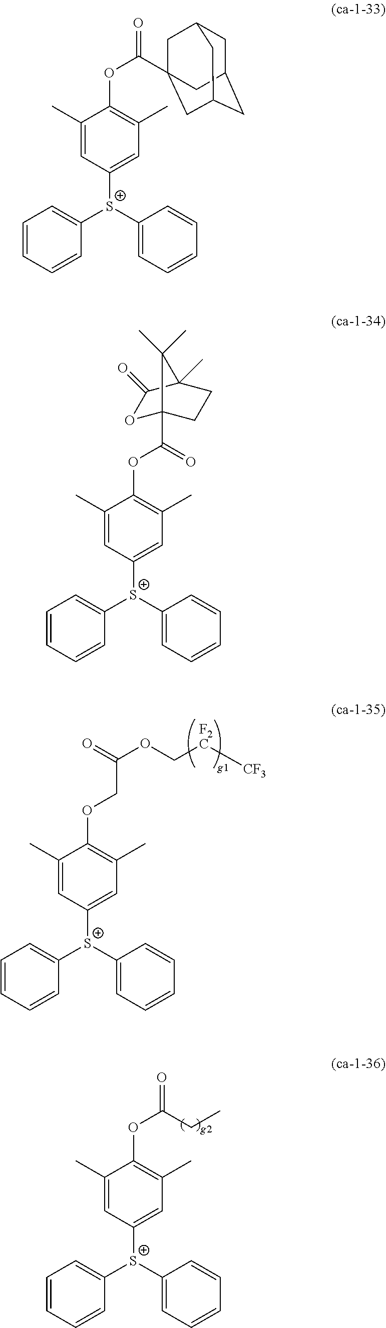

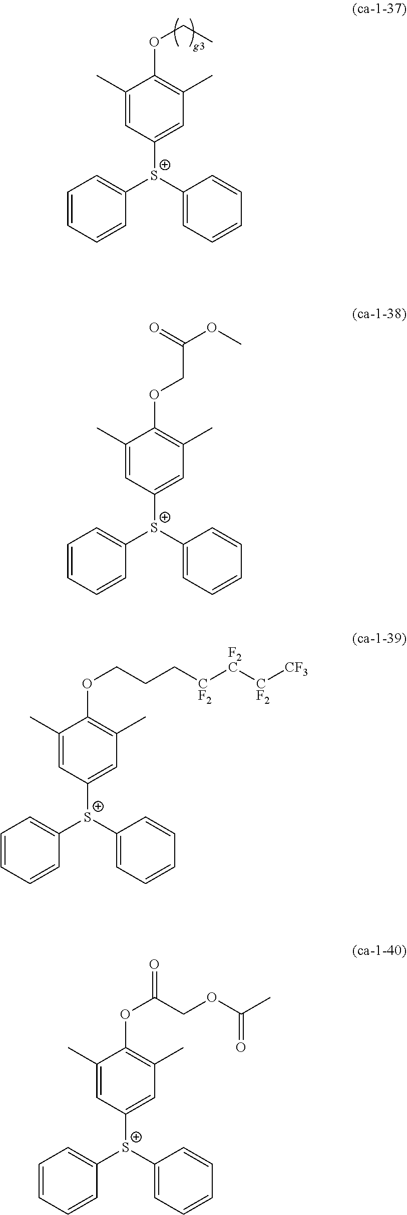

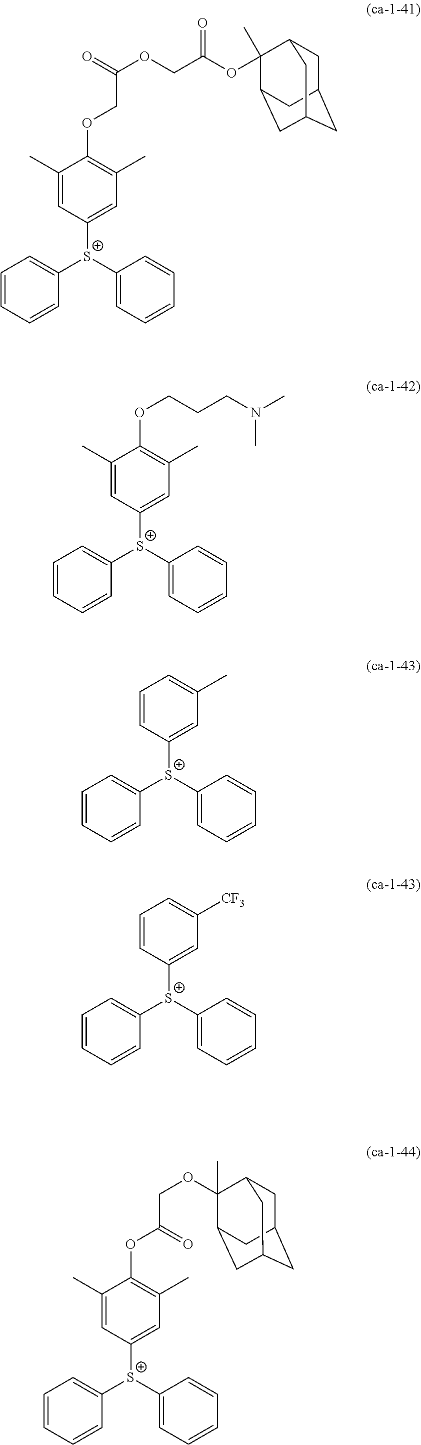

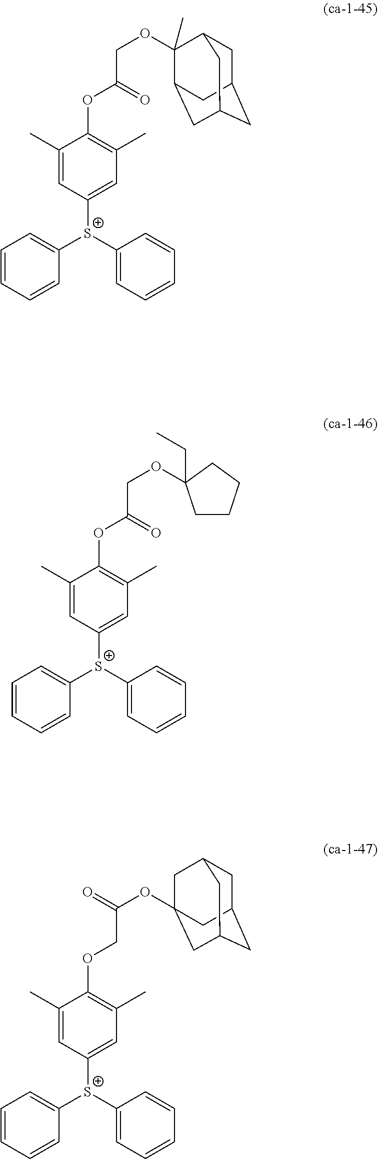

[Tn the formulae, g1, g2, and g3 represent the numbers of repetitions, g1 is an integer in a range of 1 to 5, g2 is an integer in a range of 0 to 20, and g3 is an integer in a range of 0 to 20.]

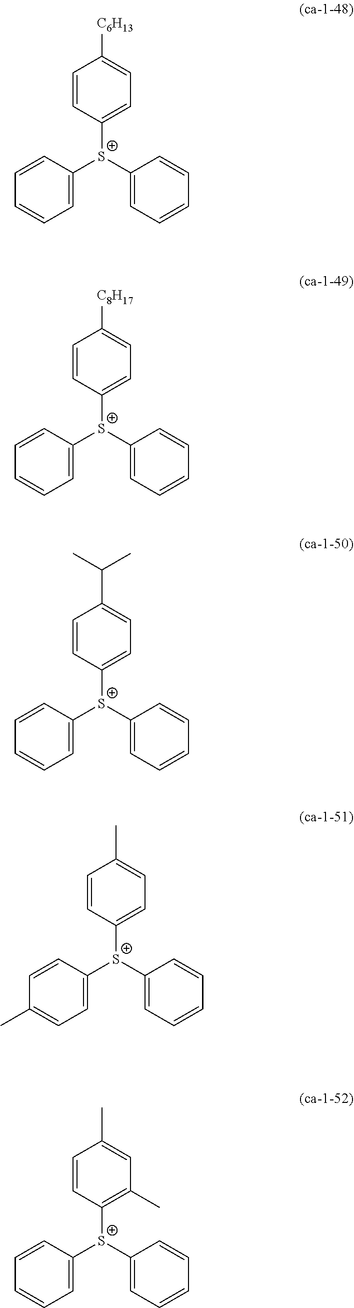

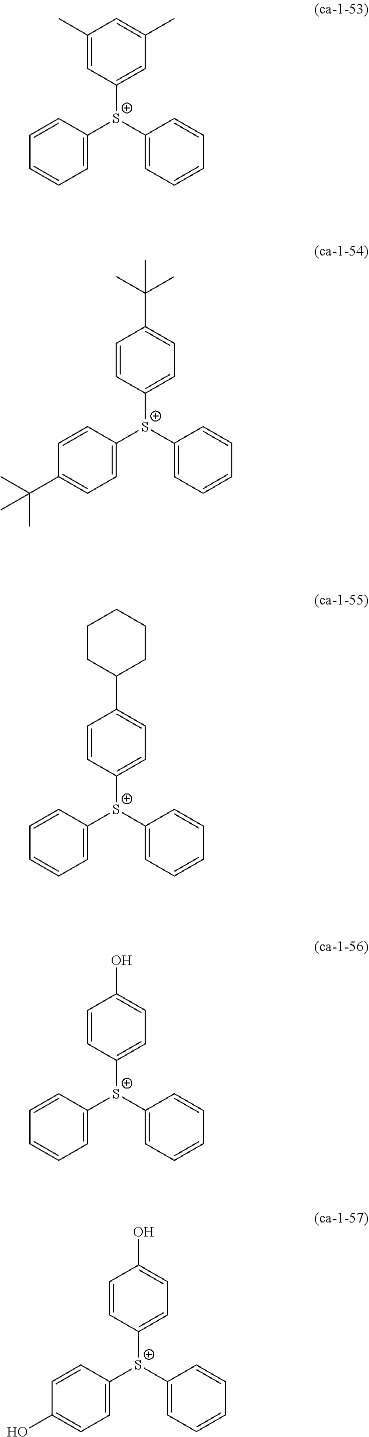 [SMILES: Cc1cc(C)cc([S+](c2ccccc2)c2ccccc2)c1 ; CC(C)(C)c1ccc([S+](c2ccccc2)c2ccc(C(C)(C)C)cc2)cc1 ; c1ccc([S+](c2ccccc2)c2ccc(C3CCCCC3)cc2)cc1 ; Oc1ccc([S+](c2ccccc2)c2ccccc2)cc1 ; Oc1ccc([S+](c2ccccc2)c2ccc(O)cc2)cc1]

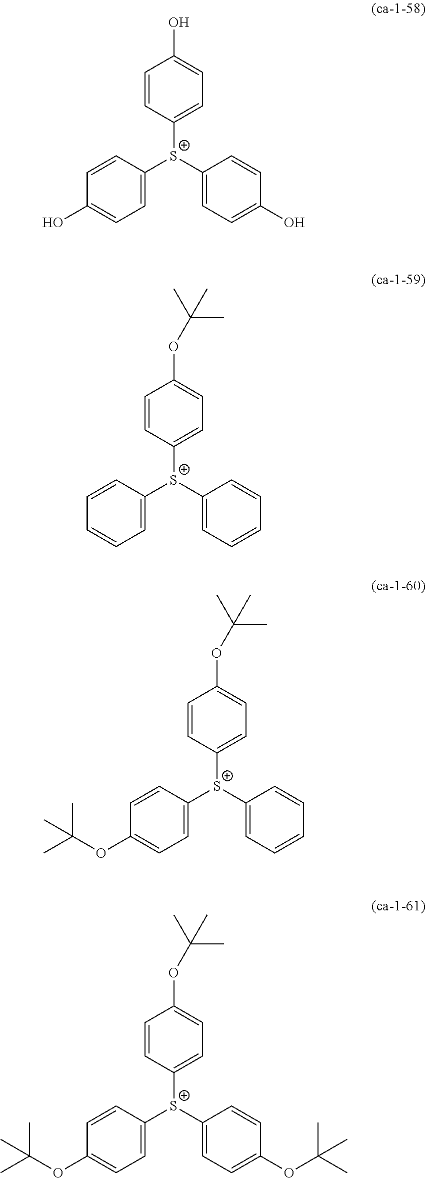 [SMILES: Oc1ccc([S+](c2ccc(O)cc2)c2ccc(O)cc2)cc1 ; CC(C)(C)Oc1ccc([S+](c2ccccc2)c2ccccc2)cc1 ; CC(C)(C)Oc1ccc([S+](c2ccccc2)c2ccc(OC(C)(C)C)cc2)cc1 ; CC(C)(C)Oc1ccc([S+](c2ccc(OC(C)(C)C)cc2)c2ccc(OC(C)(C)C)cc2)cc1]

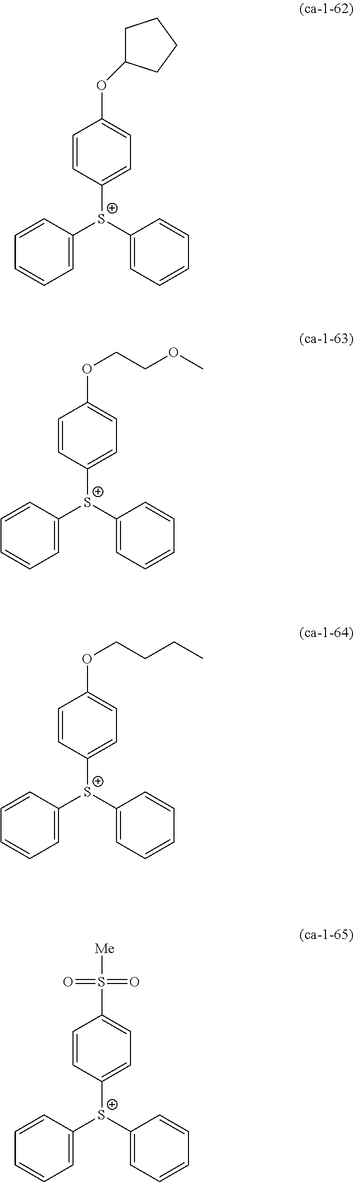 [SMILES: c1ccc([S+](c2ccccc2)c2ccc(OC3CCCC3)cc2)cc1 ; COCCOc1ccc([S+](c2ccccc2)c2ccccc2)cc1 ; CCCCOc1ccc([S+](c2ccccc2)c2ccccc2)cc1 ; CS(=O)(=O)c1ccc([S+](c2ccccc2)c2ccccc2)cc1]

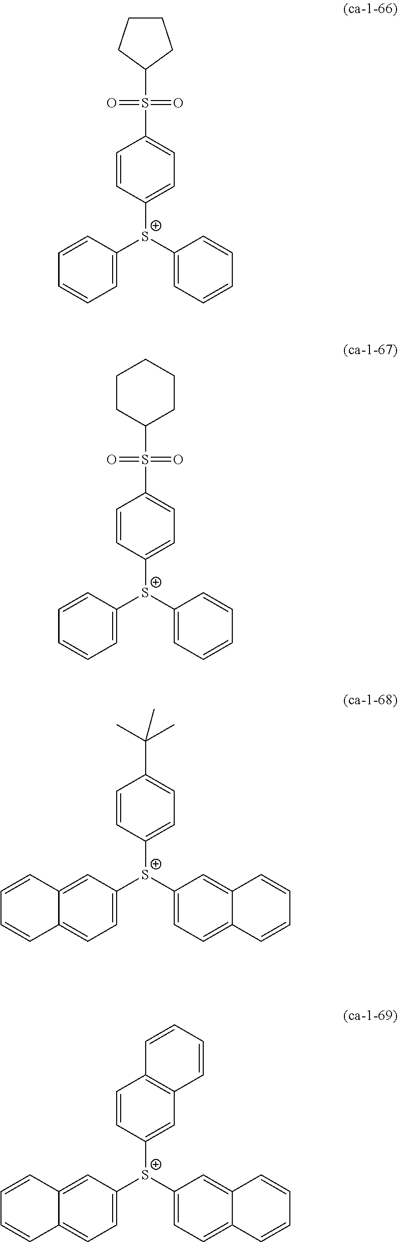 [SMILES: O=S(=O)(c1ccc([S+](c2ccccc2)c2ccccc2)cc1)C1CCCC1 ; O=S(=O)(c1ccc([S+](c2ccccc2)c2ccccc2)cc1)C1CCCCC1 ; CC(C)(C)c1ccc([S+](c2ccc3ccccc3c2)c2ccc3ccccc3c2)cc1 ; c1ccc2cc([S+](c3ccc4ccccc4c3)c3ccc4ccccc4c3)ccc2c1]

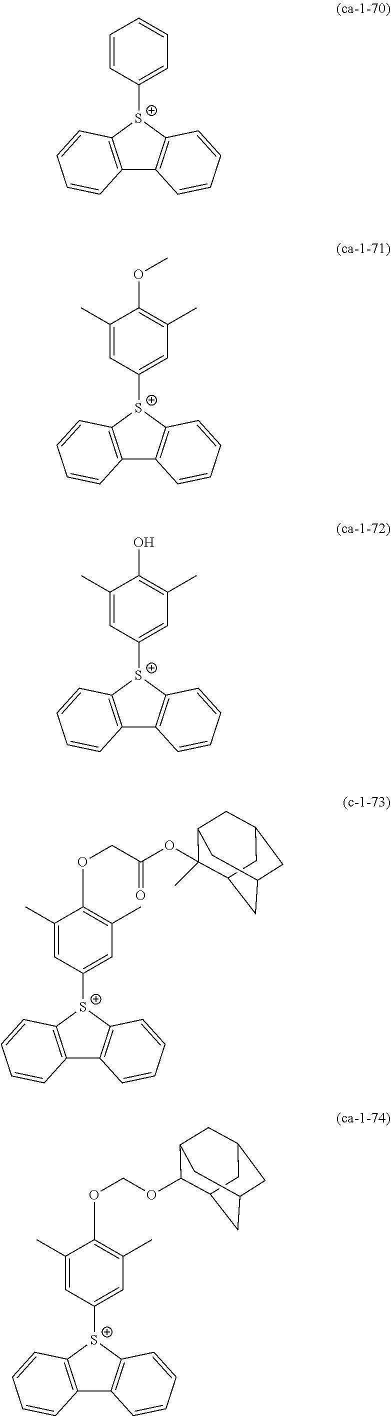 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

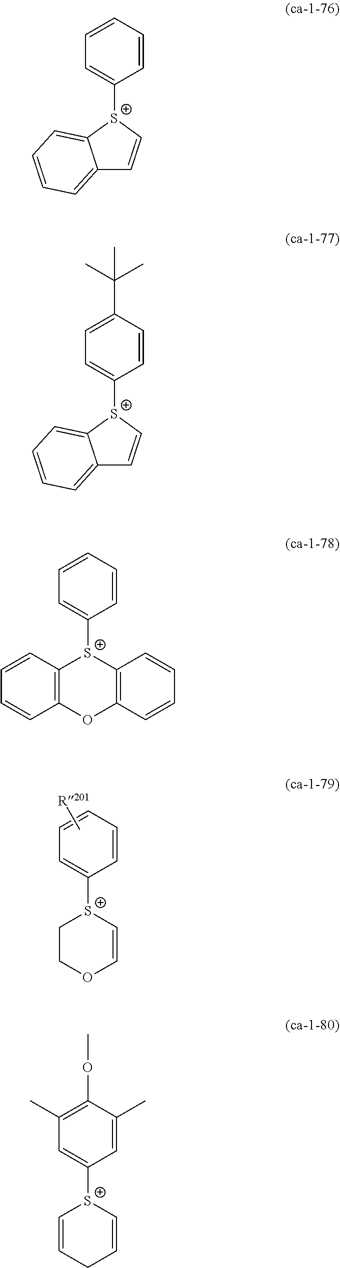

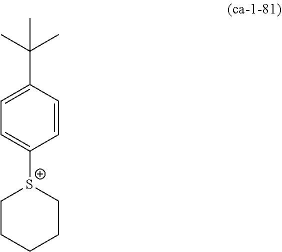 [SMILES: CC(C)(C)c1ccc([S+]2CCCCC2)cc1]

[In the formulae, R″²⁰¹ represents a hydrogen atom or a substituent, and examples of the substituent include the same groups as those for the substituents which may be contained in R²¹1 to R²⁰⁷ and R²¹⁰ to R212.]

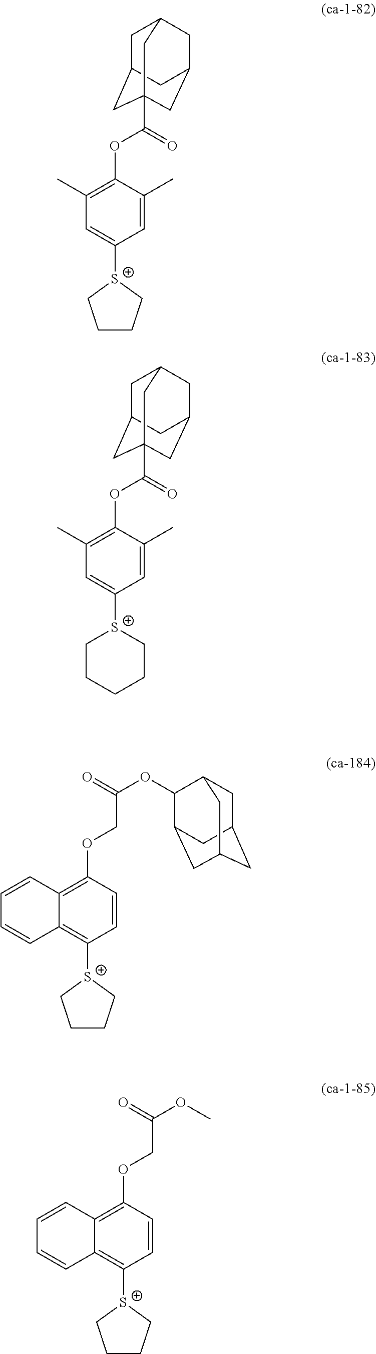 [SMILES: Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3 ; COC(=O)COc1ccc([S+]2CCCC2)c2ccccc12]

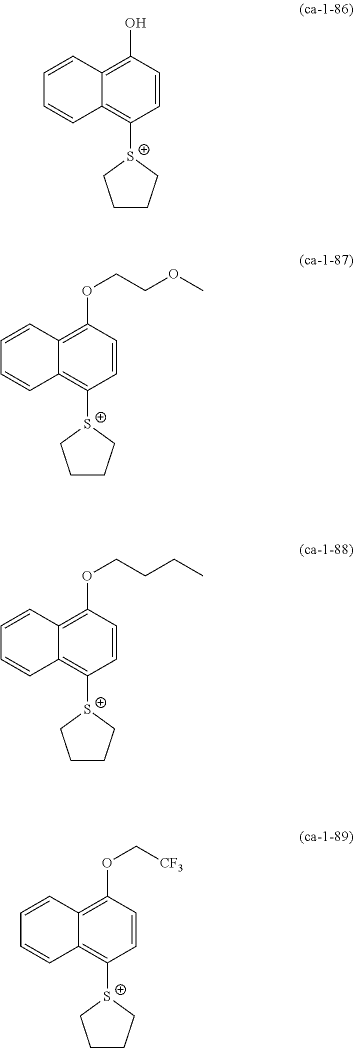 [SMILES: Oc1ccc([S+]2CCCC2)c2ccccc12 ; COCCOc1ccc([S+]2CCCC2)c2ccccc12 ; CCCCOc1ccc([S+]2CCCC2)c2ccccc12 ; FC(F)(F)COc1ccc([S+]2CCCC2)c2ccccc12]

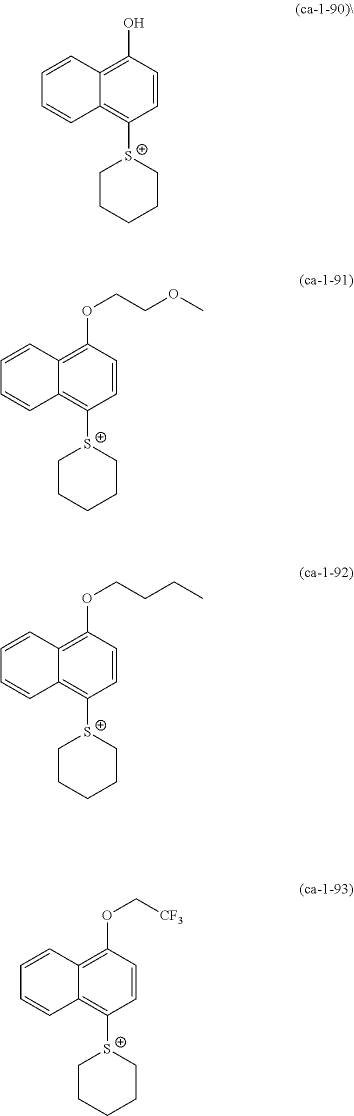 [SMILES: Oc1ccc([S+]2CCCCC2)c2ccccc12 ; COCCOc1ccc([S+]2CCCCC2)c2ccccc12 ; CCCCOc1ccc([S+]2CCCCC2)c2ccccc12 ; FC(F)(F)COc1ccc([S+]2CCCCC2)c2ccccc12]

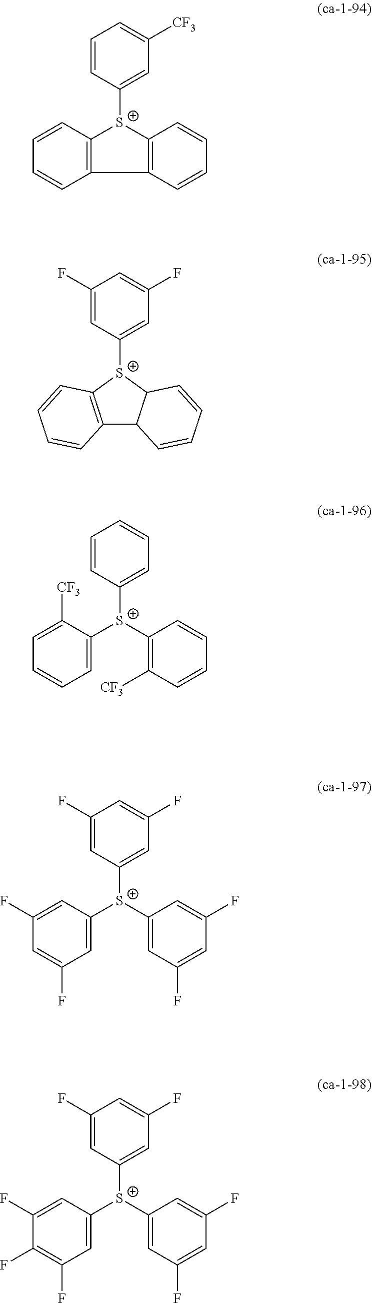 [SMILES: FC(F)(F)c1cccc(-[s+]2c3ccccc3c3ccccc32)c1 ; Fc1cc(F)cc([S+]2c3ccccc3C3C=CC=CC32)c1 ; FC(F)(F)c1ccccc1[S+](c1ccccc1)c1ccccc1C(F)(F)F ; Fc1cc(F)cc([S+](c2cc(F)cc(F)c2)c2cc(F)cc(F)c2)c1 ; Fc1cc(F)cc([S+](c2cc(F)cc(F)c2)c2cc(F)c(F)c(F)c2)c1]

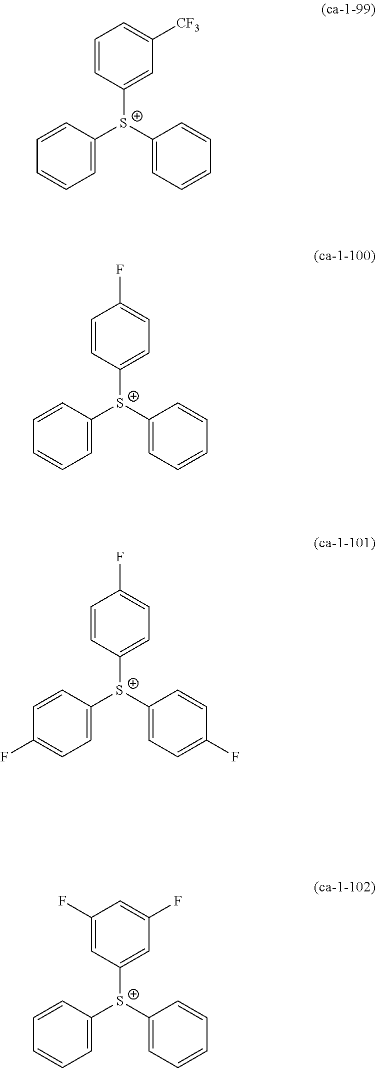 [SMILES: FC(F)(F)c1cccc([S+](c2ccccc2)c2ccccc2)c1 ; Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1]

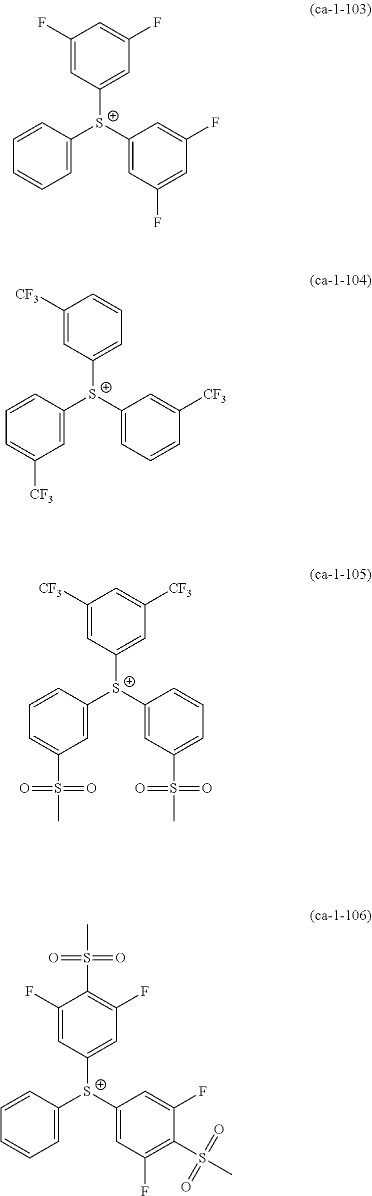 [SMILES: Fc1cc(F)cc([S+](c2ccccc2)c2cc(F)cc(F)c2)c1 ; FC(F)(F)c1cccc([S+](c2cccc(C(F)(F)F)c2)c2cccc(C(F)(F)F)c2)c1 ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F]

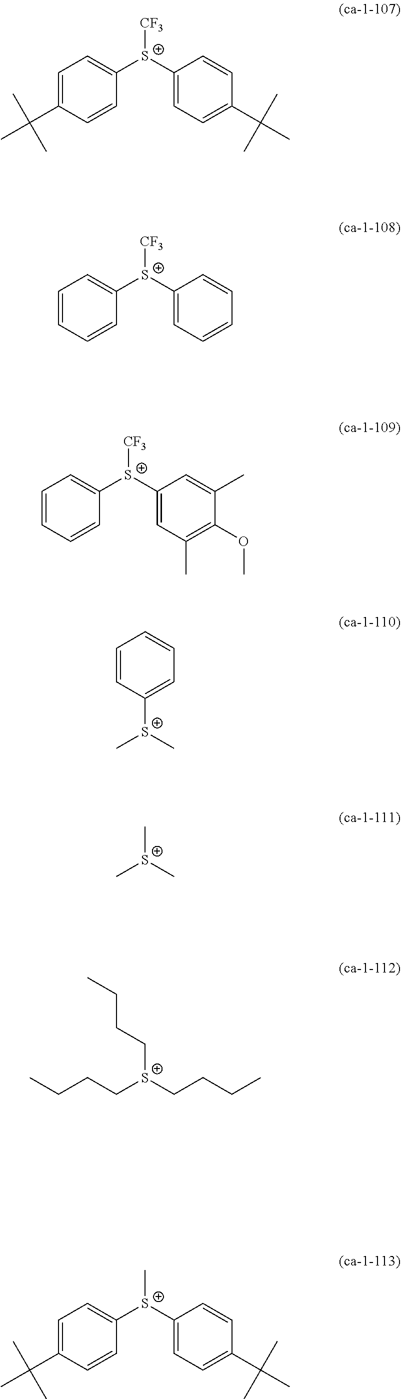

Specific examples of suitable cations represented by General Formula (ca-2) include a diphenyliodonium cation and a bis(4-tert-butylphenyl)iodonium cation.

Specific examples of the suitable cations represented by General Formula (ca-3) include cations represented by each of General Formulae (ca-3-1) to (ca-3-6) shown below.

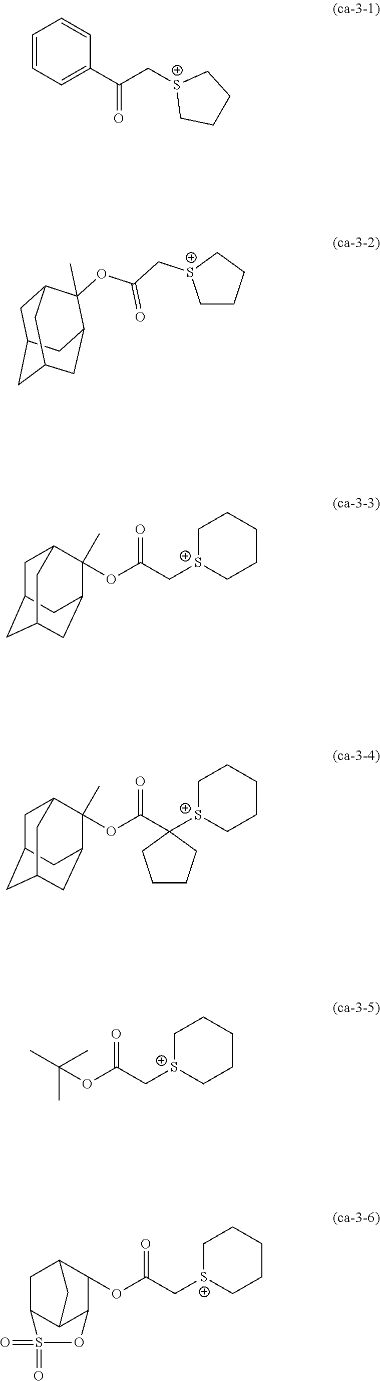

Among the above, as the cation moiety ((M^{m+})_{1/m}), a cation represented by General Formula (ca-1) is preferable.

In addition, from the viewpoint of improving the decomposability of the cation moiety, it is preferable that R²⁰¹ to R²⁰³ in the cation represented by General Formula (ca-1) each independently represent an aryl group which may have a substituent and have at least one electron-withdrawing group as the substituent, or that R²⁰¹ to R²⁰³ each independently represent an aryl group which may have a substituent and any two of R²⁰¹ to R²⁰³ be bonded to each other to form a ring together with the sulfur atom in the formula, and it is more preferable that R²⁰¹ to R²⁰³ in the cation represented by General Formula (ca-1) each independently represent an aryl group which may have a substituent and have at least one electron-withdrawing group as the substituent.

The electron-withdrawing group may be one kind or may be two or more kinds.

In addition, the electron-withdrawing group may be a monovalent electron-withdrawing group or a divalent electron-withdrawing group.

Specific examples of the electron-withdrawing group include an acyl group, a halogen atom, a halogenated alkyl group, a halogenated alkoxy group, a halogenated aryloxy group, a halogenated alkylamino group, a halogenated alkylthio group, a cyano group, a nitro group, a dialkylphosphono group, a diarylphosphono group, an alkylsulfonyl group, a cycloalkylsulfonyl group, an arylsulfonyl group, a sulfonyloxy group, an acylthio group, a sulfamoyl group, a thiocyanate group, and a thiocarbonyl group.

Among the above, the electron-withdrawing group is preferably a fluorine atom, a fluorinated alkyl group, or a cycloalkylsulfonyl group, more preferably a fluorine atom or a cycloalkylsulfonyl group, and still more preferably a fluorine atom.

The cation moiety ((M^{m+})_{1/m}) is preferably a cation represented by any one of Chemical Formulae (ca-1-65) to (ca-1-67), (ca-1-70), or (ca-1-94) to (ca-1-106), more preferably a cation represented by any one of Chemical Formulae (ca-1-67), (ca-1-70), or (ca-1-103), and still more preferably a cation represented by Chemical Formula (ca-1-103).

In the resist composition according to the present embodiment, among the above, the component (D0) is preferably a compound represented by General Formula (d0-1).

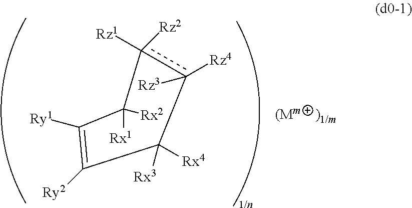

[In the formula. Rx¹ to Rx4 each independently represent a hydrocarbon group which may have a substituent or a hydrogen atom, or two or more of Rx¹ to Rx⁴ may be bonded to each other to form a ring structure. Ry¹ and Ry² each independently represent a hydrocarbon group which may have a substituent or a hydrogen atom, or may be bonded to each other to form a ring structure.

[symbol]

The above double line of the dotted line and the straight line represents a double bond or a single bond. Where valence permits, Rz¹ to Rz⁴ each independently represent a hydrocarbon group which may have a substituent or a hydrogen atom, or two or more of Rz¹ to Rz⁴ may be bonded to each other to form a ring structure. However, at least two or more of Rx¹ to Rx⁴, at least Ry¹ and Ry², or at least two or more of Rz¹ to Rz⁴ are bonded to each other to form an aromatic ring. In addition, at least one among Rx^{I} to Rx⁴, Ry¹ and Ry², and Rz¹ to Rz⁴ has an anion group represented by General Formula (d0-r-an1), where the entire anion moiety is an n-valent anion, In addition, at least one among Rx¹ to Rx⁴, Ry¹ and Ry², and Rz¹ to Rz⁴ includes a hydrocarbon group having a bromine atom or a hydrocarbon group having an iodine atom. n represents an integer of 1 or more. M^{m+} represents an m-valent organic cation, where m represents an integer of 1 or more.]

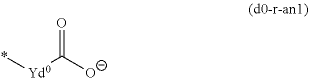

[In the formula, Yd⁰ represents a divalent linking group or a single bond. * represents a bonding site.]

The anion moiety of the compound represented by General Formula (d0-1) is the same as the anion represented by General Formula (d0-an0).

Yd⁰ in General Formula (d0-r-an1) is preferably a divalent linking group containing an ester bond or a divalent linking group containing an ether bond, and more preferably a linking group represented by General Formulae (y-d0-1) or (y-d0-2).

The cation moiety of the compound represented by General Formula (d0-1) is the same as the cation moiety of the compound represented by General Formula (d0). The cation moiety of the compound represented by General Formula (d0-1) is the same as the cation moiety of the compound represented by General Formula (d0). Among the above, a cation represented by General Formula (ca-1) is preferable. In addition, from the viewpoint of improving the decomposability of the cation moiety, it is preferable that R²⁰¹ to R²⁰³ in the cation represented by General Formula (ca-1) each independently represent an aryl group which may have a substituent and have at least one electron-withdrawing group as the substituent, or that R²⁰¹ to R²⁰³ each independently represent an aryl group which may have a substituent and any two of R²⁰¹ to R²⁰³ be bonded to each other to form a ring together with the sulfur atom in the formula, and it is more preferable that R²⁰¹ to R²⁰³ in the cation represented by General Formula (ca-1) each independently represent an aryl group which may have a substituent and have at least one electron-withdrawing group as the substituent.

Specific examples of the component (D0) are shown below but are not limited thereto.

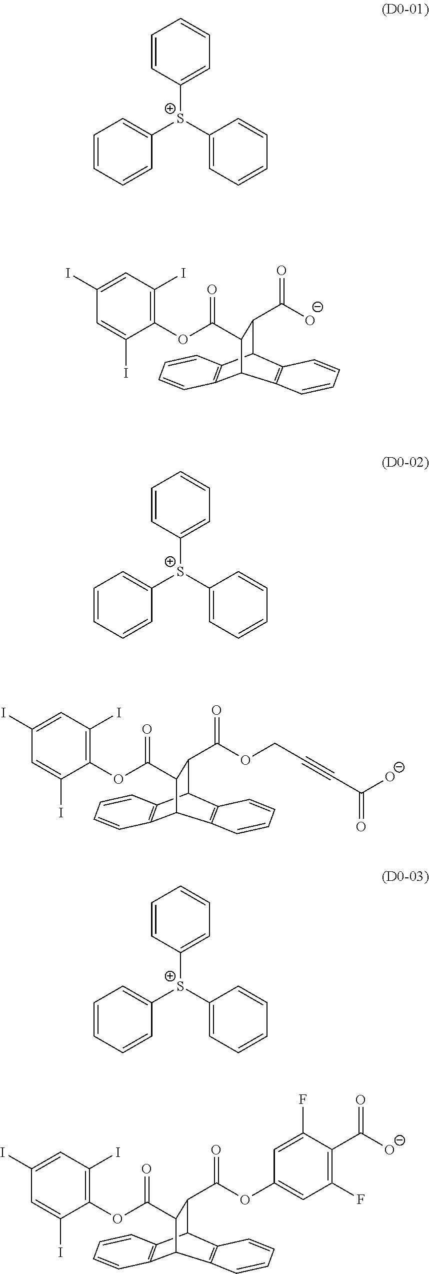 [SMILES: c1ccc([S+](c2ccccc2)c2ccccc2)cc1 ; O=C([O-])[C@H]1C2c3ccccc3C(c3ccccc32)[C@@H]1C(=O)Oc1c(I)cc(I)cc1I ; c1ccc([S+](c2ccccc2)c2ccccc2)cc1 ; O=C([O-])C#CCOC(=O)[C@H]1C2c3ccccc3C(c3ccccc32)[C@@H]1C(=O)Oc1c(I)cc(I)cc1I ; c1ccc([S+](c2ccccc2)c2ccccc2)cc1 ; O=C([O-])c1c(F)cc(OC(=O)[C@H]2C3c4ccccc4C(c4ccccc43)[C@@H]2C(=O)Oc2c(I)cc(I)cc2I)cc1F]

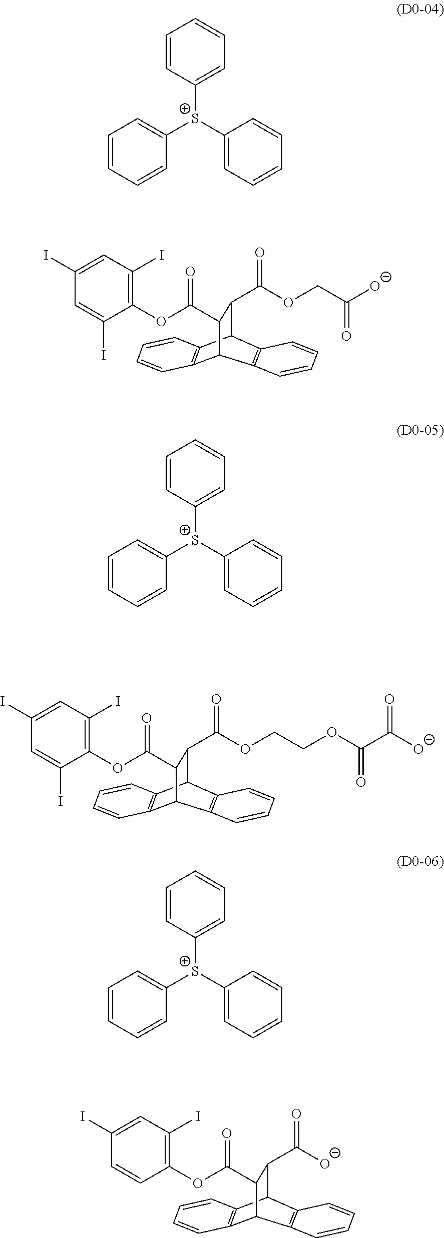 [SMILES: c1ccc([S+](c2ccccc2)c2ccccc2)cc1 ; O=C([O-])COC(=O)[C@H]1C2c3ccccc3C(c3ccccc32)[C@@H]1C(=O)Oc1c(I)cc(I)cc1I ; c1ccc([S+](c2ccccc2)c2ccccc2)cc1 ; O=C([O-])C(=O)OCCOC(=O)[C@H]1C2c3ccccc3C(c3ccccc32)[C@@H]1C(=O)Oc1c(I)cc(I)cc1I ; c1ccc([S+](c2ccccc2)c2ccccc2)cc1 ; O=C([O-])[C@H]1C2c3ccccc3C(c3ccccc32)[C@@H]1C(=O)Oc1ccc(I)cc1I]

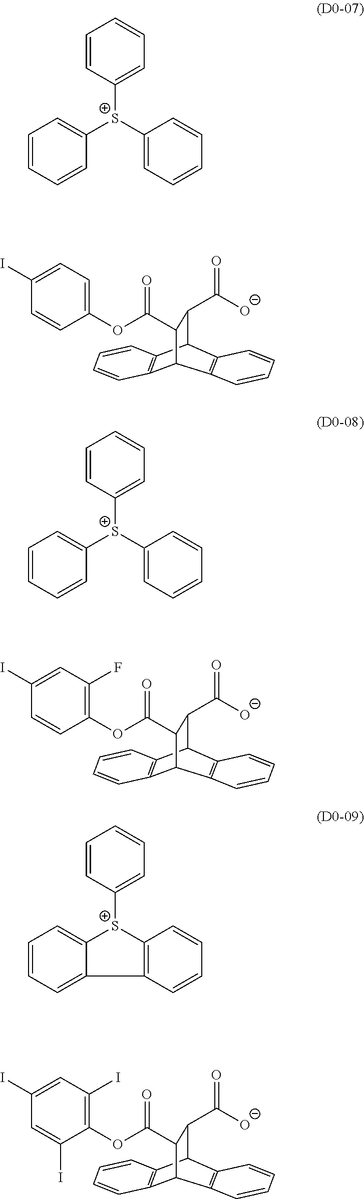 [SMILES: c1ccc([S+](c2ccccc2)c2ccccc2)cc1 ; O=C([O-])[C@H]1C2c3ccccc3C(c3ccccc32)[C@@H]1C(=O)Oc1ccc(I)cc1 ; c1ccc([S+](c2ccccc2)c2ccccc2)cc1 ; O=C([O-])[C@H]1C2c3ccccc3C(c3ccccc32)[C@@H]1C(=O)Oc1ccc(I)cc1F ; c1ccc(-[s+]2c3ccccc3c3ccccc32)cc1 ; O=C([O-])[C@H]1C2c3ccccc3C(c3ccccc32)[C@@H]1C(=O)Oc1c(I)cc(I)cc1I]

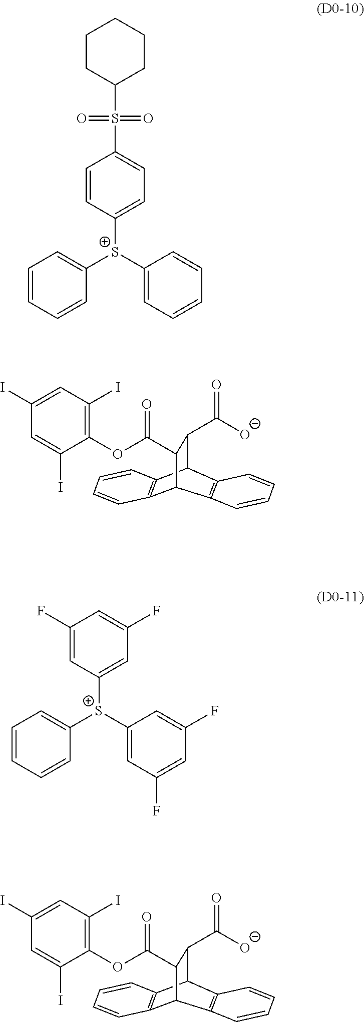 [SMILES: O=[S@SP2](=O)(c1ccc([S+](c2ccccc2)c2ccccc2)cc1)C1CCCCC1 ; O=C([O-])[C@H]1C2c3ccccc3C(c3ccccc32)[C@@H]1C(=O)Oc1c(I)cc(I)cc1I ; Fc1cc(F)cc([S+](c2ccccc2)c2cc(F)cc(F)c2)c1 ; O=C([O-])[C@H]1C2c3ccccc3C(c3ccccc32)[C@@H]1C(=O)Oc1c(I)cc(I)cc1I]

In the resist composition according to the present embodiment, the component (D0) may be used alone or may be used in a combination of two or more kinds thereof.

The content of the component (D0) in the resist composition according to the present embodiment is preferably in a range of 1 to 30 parts by mass, more preferably in a range of 3 to 20 parts by mass, and still more preferably in a range of 5 to 15 parts by mass with respect to 100 parts by mass of the component (A).

In a case where the content of the component (D0) is equal to or larger than the lower limit value of the above-described preferred range, sensitivity and CDU are further improved in the resist pattern formation. On the other hand, in a case where it is equal to or smaller than the upper limit value of the preferred range, satisfactory sensitivity can be maintained.

The component (D) in the resist composition according to the present embodiment may contain a base component other than the above-described component (D0).

Examples of the base component other than the component (D0) include a photodecomposable base (D1) having acid diffusion controllability (hereinafter, referred to as a “component (D1)”) which is lost by being decomposed upon exposure and a nitrogen-containing organic compound (D2) (hereinafter, referred to as a “component (D2)”) which does not correspond to the component (Dl).

### In Regard to Component (D1)

The component (D1) is not particularly limited as long as it is decomposed upon exposure and loses the acid diffusion controllability. The component (D1) is preferably one or more compounds selected from the group consisting of a compound represented by General Formula (d11˜) (hereinafter, referred to as a “component (d11˜)”), a compound represented by General Formula (d1-2) (hereinafter, referred to as a “component (d1-2)”), and a compound represented by General Formula (d1-3) (hereinafter, referred to as a “component (d1-3)”).

At exposed portions of the resist film, the components (d1-1) to (d1-3) are decomposed and then lose the acid diffusion controllability (basicity), and thus they cannot act as a quencher, whereas they act as a quencher at unexposed portions of the resist film.

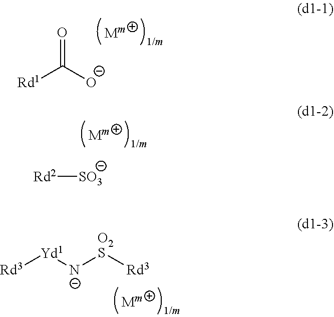

[In the formulae, Rd¹ to Rd⁴ represent a cyclic group which may have a substituent, a chain-like alkyl group which may have a substituent, or a chain-like alkenyl group which may have a substituent. Here, no fluorine atom is bonded to the carbon atom adjacent to the S atom in Rd² of General Formula (d1-2). Yd¹ represents a single bond or a divalent linking group. m represents an integer of 1 or more, and M^{m+}'s each independently represent an m-valent organic cation.]

{Component (d1-1)}

### Anion Moiety

In General Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that Rd⁰ represent an aromatic hydrocarbon group which may have a substituent, an aliphatic cyclic group which may have a substituent, or a chain-like alkyl group which may have a substituent. Examples of the substituent which may be contained in these groups include a hydroxyl group, an oxo group, an alkyl group, an aryl group, a fluorine atom, a fluorinated alkyl group, lactone-containing cyclic groups represented by any of General Formulae (a2-r-1) to (a2-r-7), an ether bond, an ester bond, and a combination thereof.

In a case where an ether bond or an ester bond is contained as the substituent, the substituent may be bonded through an alkylene group, and the substituent in this case is preferably a linking group represented by any of General Formulae (y-a1-1) to (y-a1-5). It is noted that in a case where the aromatic hydrocarbon group, the aliphatic cyclic group, or the chain-like alkyl group, as Rd¹, has a linking group represented by any of General Formulae (y-a1-1) to (y-a1-7) as a substituent, in General Formulae (y-a1-1) to (y-a1-7), the group that is bonded to a carbon atom constituting the aromatic hydrocarbon group, the aliphatic cyclic group, or the chain-like alkyl group, as Rd¹, in General Formula (d3-1) is V′¹⁰¹ in General Formulae (y-a1-1) to (y-a1-7).

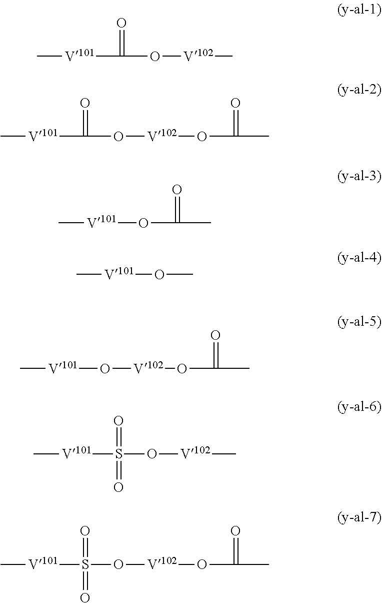

[In the formulae, V′¹⁰¹ represents a single bond or an alkylene group having 1 to 5 carbon atoms, and V′¹⁰² represents a divalent saturated hydrocarbon group having 1 to 30 carbon atoms.]

The divalent saturated hydrocarbon group as V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group as V′¹⁰¹ and V′¹⁰² may be a linear alkylene group or a branched alkylene group, and a linear alkylene group is preferable.

Specific examples of the alkylene group as V′¹⁰¹ and V′¹⁰² include a methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, or —C(CH₂CH₃)₂—; an ethylene group [—CH₂CH₂—]; an alkylethylene group such as —CH(CH₃)CH₂—, —CH(CH₃)CH(CH₃)—, —C(CH₃)₂CH₂—, or —CH(CH₂CH₃)CH₂—; a trimethylene group (n-propylene group) [—CH₂CH₂CH₂—]; an alkyltrimethylene group such as —CH(CH₃)CH₂CH₂— or —CH₂CH(CH₃)CH₂—; a tetramethylene group [—CH₂CH₂CH₂CH₂—]; an alkyltetramethylene group such as —CH(CH₃)CH₂CH₂CH₂— or —CH₂CH(CH₃)CH₂CH₂—; and a pentamethylene group [—CH₂CH₂CH₂CH₂CH₂—].

Further, some methylene groups in the alkylene group as V′¹⁰¹ and V′¹⁰² may be substituted with a divalent aliphatic cyclic group having 5 to 10 carbon atoms. As the aliphatic cyclic group, a divalent group in which one hydrogen atom has been further removed from the cyclic aliphatic hydrocarbon group (a monocyclic aliphatic hydrocarbon group or a polycyclic aliphatic hydrocarbon group) as Ra′³ in General Formula (a1-r-1) is preferable, and a cyclohexylene group, a 1,5-adamantylene group, or a 2,6-adamantylene group is more preferable.

Suitable examples of the aromatic hydrocarbon group include a phenyl group, a naphthyl group, and a polycyclic structure (a polycyclic structure consisting of a bicyclooctane skeleton and a ring structure other than the bicyclooctane skeleton) including a bicyclooctane skeleton.

The aliphatic cyclic group is preferably a group obtained by removing one or more hydrogen atoms from a polycycloalkane such as adamantane, norbornane, isobornane, tricyclodecane, or tetracyclododecane.

The chain-like alkyl group preferably has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group, and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group preferably has 1 to 11 carbon atoms, more preferably has 1 to 8 carbon atoms, and still more preferably has 1 to 4 carbon atoms. The fluorinated alkyl group may contain an atom other than a fluorine atom. Examples of the atom other than a fluorine atom include an oxygen atom, a sulfur atom, and a nitrogen atom.

Specific examples of the preferred anion moiety for the component (d1-1) are shown below.

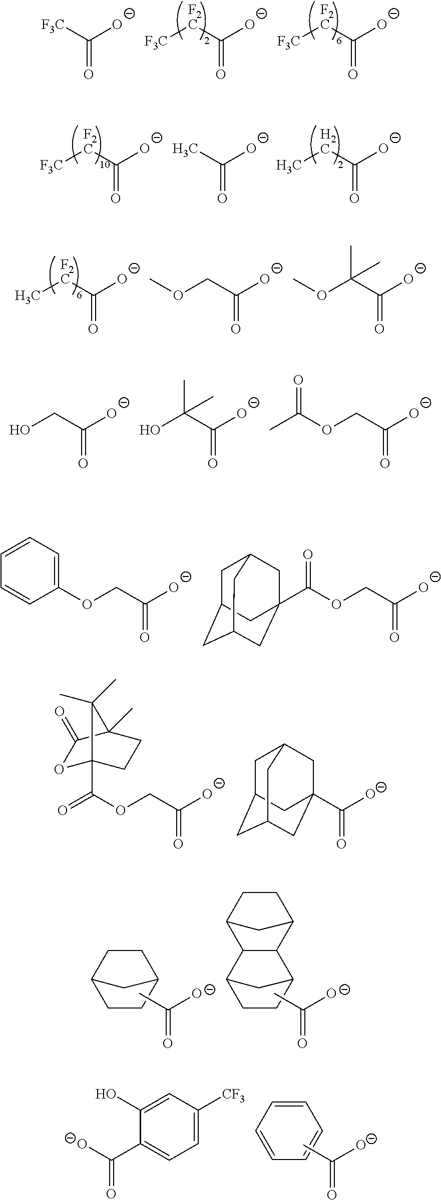

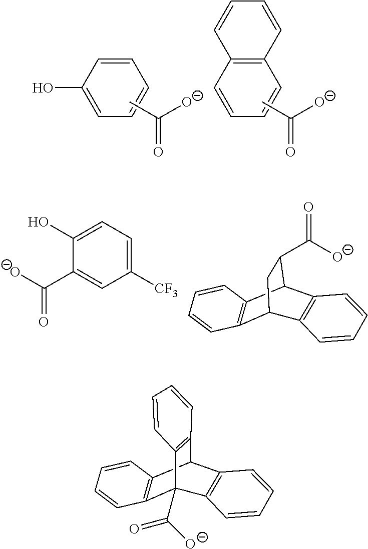

### Cation Moiety

In General Formula (d1-1), M^{m+} represents an m-valent organic cation.

The suitable examples of the organic cation as M^{m+} include the same cations as the cations represented by each of General Formulae (ca-1) to (ca-3), the cation represented by General Formula (ca-1) is preferable, and cations represented by each of General Formulae (ca-1-1) to (ca-1-113) are more preferable.

The component (d1-1) may be used alone or in a combination of two or more kinds thereof.

{Component (d1-2)}

### Anion Moiety

In General Formula (d11-2), Rd² represents a cyclic group which may have a substituent, a chain-like alkyl group which may have a substituent, or a chain-like alkenyl group which may have a substituent, and examples thereof include the same groups as those for R′²⁰¹.

Here, no fluorine atom is bonded to the carbon atom adjacent to the S atom in Rd² (the carbon atom is not substituted with fluorine). As a result, the anion of the component (d1-2) becomes an appropriately weak acid anion, thereby improving the quenching ability of the component (D).

Rd² is preferably a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably has 3 to 10 carbon atoms.

The aliphatic cyclic group is more preferably a group (which may have a substituent) obtained by removing one or more hydrogen atoms from adamantane, norbornane, isobornane, tricyclodecane, tetracyclododecane, or the like; and a group obtained by removing one or more hydrogen atoms from camphor.

The hydrocarbon group as Rd² may have a substituent, and examples of the substituent include the same groups as those for the substituent which may be contained in the hydrocarbon group (such as an aromatic hydrocarbon group, an aliphatic cyclic group, or a chain-like alkyl group) as Rd¹ in General Formula (d1-1).

Among the above, the anion moiety of the component (d1-2) is preferably a camphorsulfonic acid anion.

Specific examples of the preferred anion moiety for the component (d1-2) are shown below.

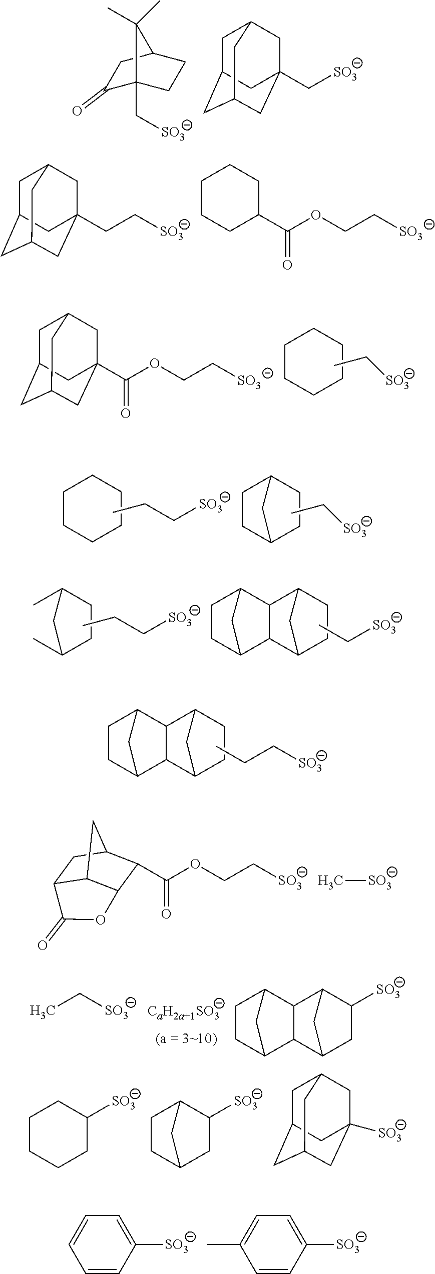

### Cation Moiety

In General Formula (d1-2), M^{m+} represents an m-valent organic cation and has the same definition as M^{m+} in General Formula (d1-1).

The component (d1-2) may be used alone or in a combination of two or more kinds thereof.

{Component (d1-3)}

### Anion Moiety

In General Formula (d11-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In General Formula (d11-3), Rd⁴ represents a cyclic group which may have a substituent, a chain-like alkyl group which may have a substituent, or a chain-like alkenyl group which may have a substituent, and examples thereof include the same groups as those for R²⁰¹.

Among them, an alkyl group which may have a substituent, an alkoxy group which may have a substituent, an alkenyl group which may have a substituent, or a cyclic group which may have a substituent is preferable.

It is preferable that the alkyl group as Rd⁴ be a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ be an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰. Among these, an alicyclic group in which one or more hydrogen atoms have been removed from a cycloalkane such as cyclopentane, cyclohexane, adamantane, norbornane, isobornane, tricyclodecane or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are enhanced. Further, in a case where Rd⁴ represents an aromatic group, the resist composition has excellent light absorption efficiency in lithography using EUV or the like as a light source for exposure, and thus the sensitivity and lithography characteristics are enhanced.

In General Formula (d1-3), Ydⁱ represents a single bond or a divalent linking group.

The divalent linking group as Yd⁰ is not particularly limited, and examples thereof include a divalent hydrocarbon group (an aliphatic hydrocarbon group or an aromatic hydrocarbon group) which may have a substituent and a divalent linking group having a hetero atom. Examples of the divalent linking groups are the same as those for the divalent hydrocarbon group which may have a substituent and the divalent linking group having a hetero atom described in the section of the divalent linking group as Ya²¹ in General Formula (a2-1).

It is preferable that Yd^{t} represent a carbonyl group, an ester bond, an amide bond, an alkylene group, or a combination thereof. The alkylene group is more preferably a linear or branched alkylene group, and still more preferably a methylene group or an ethylene group.

Specific examples of the preferred anion moiety for the component (d1-3) are shown below.

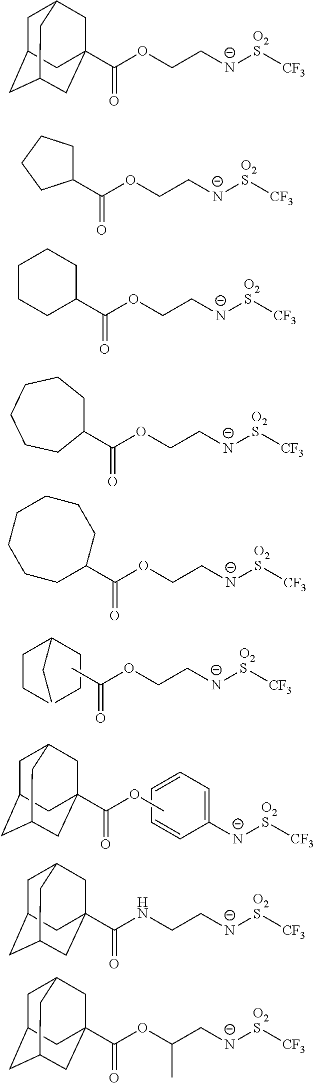

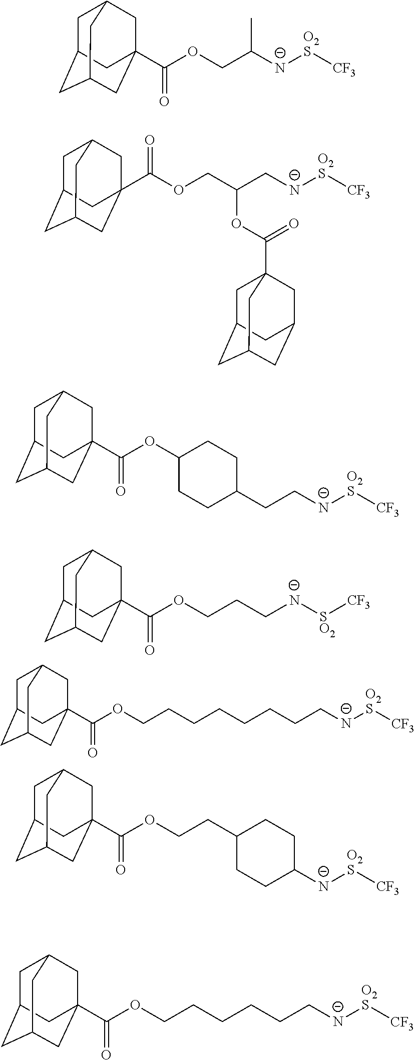

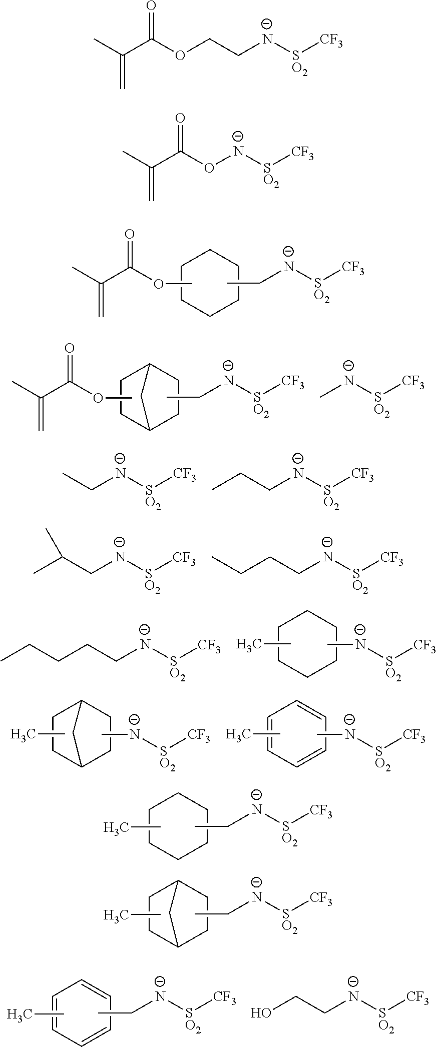

### Cation Moiety

In General Formula (d1-3), M^{m+} represents an m-valent organic cation and has the same definition as M^{m+} in General Formula (d1-1).

The component (d1-3) may be used alone or in a combination of two or more kinds thereof.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 10 parts by mass and more preferably in a range of 0.5 to 5 parts by mass with respect to 100 parts by mass of the component (A1).

In a case where the content of the component (Dl) is equal to or larger than the preferred lower limit value, excellent lithography characteristics and an excellent resist pattern shape are easily obtained. On the other hand, in a case where the content thereof is equal to or smaller than the upper limit value, satisfactory sensitivity can be maintained and the throughput is also excellent.

### Method of Producing Component (D1):

The methods of producing the components (d1-1) and (d1-2) described above are not particularly limited, and the components (d1-1) and (d1-2) can be produced by conventionally known methods.

Further, the method of producing the component (d1-3) is not particularly limited, and the component (d1-3) can be produced, for example, in the same manner as disclosed in United States Patent Application, Publication No. 2012-0149916.

### In Regard to Component (D2)

The component (D) may contain a nitrogen-containing organic compound component (hereinafter, referred to as a “component (D2)”) which does not correspond to the above-described component (D1).

The component (D2) is not particularly limited as long as it acts as an acid diffusion control agent and does not correspond to the component (D1), and any known compound may be used. Among the above, aliphatic amines are preferable, and among the aliphatic amines, a secondary aliphatic amine or a tertiary aliphatic amine is more preferable.

An aliphatic amine is an amine having one or more aliphatic groups, and the aliphatic groups preferably have 1 to 12 carbon atoms.

Examples of the aliphatic amine include amines in which at least one hydrogen atom of ammonia NH₃ has been substituted with an alkyl group or hydroxyalkyl group having 12 or less carbon atoms (alkyl amines or alkyl alcohol amines), and cyclic amines.

Specific examples of the alkyl amine and the alkyl alcohol amine include monoalkyl amines such as n-hexyl amine, n-heptyl amine, n-octyl amine, n-nonyl amine, and n-decyl amine; dialkyl amines such as diethyl amine, di-n-propyl amine, di-n-heptyl amine, di-n-octyl amine, and dicyclohexyl amine; trialkyl amines such as trimethyl amine, triethyl amine, tri-n-propyl amine, tri-n-butyl amine, tri-n-hexyl amine, tri-n-pentyl amine, tri-n-heptyl amine, tri-n-octyl amine, tri-n-nonyl amine, tri-n-decyl amine, and tri-n-dodecyl amine; and alkyl alcohol amines such as diethanol amine, triethanol amine, diisopropanol amine, triisopropanol amine, di-n-octanol amine, and tri-n-octanol amine. Among these, trialkyl amines of 5 to 10 carbon atoms are preferable, and tri-n-pentyl amine and tri-n-octyl amine are particularly preferable.

Examples of the cyclic amine include heterocyclic compounds containing a nitrogen atom as a hetero atom. The heterocyclic compound may be a monocyclic compound (aliphatic monocyclic amine), or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxy)ethoxy)ethyl}amine, tris{2-(1-ethoxypropoxy)ethyl}amine, tris[2-{2-(2-hydroxyethoxy)ethoxy}ethyl]amine and triethanol amine triacetate, and triethanol amine triacetate is preferable.

In addition, as the component (D2), an aromatic amine may be used.

Examples of aromatic amines include 4-dimethylaminopyridine, pyrrole, indole, pyrazole, imidazole, and derivatives thereof, tribenzylamine, 2,6-diisopropylaniline, N-tert-butoxycarbonylpyrrolidine, and 2,6-di-tert-butylpyridine.

Among the above, the component (D2) is preferably an alkyl amine and more preferably a trialkyl amine having 5 to 10 carbon atoms.

The component (D2) may be used alone or in a combination of two or more kinds thereof.

In a case where the resist composition contains the component (D2), the content of the component (D2) in the resist composition is preferably in a range of 0.01 to 5 parts by mass, more preferably in a range of 0.1 to 5 parts by mass, and still more preferably in a range of 0.5 to 5 parts by mass with respect to 100 parts by mass of the component (A1).

In a case where the content of the component (D2) is equal to or larger than the preferred lower limit value, excellent lithography characteristics and an excellent resist pattern shape are easily obtained. On the other hand, in a case where the content thereof is equal to or smaller than the upper limit value, satisfactory sensitivity can be maintained and the throughput is also excellent.

The content of the component (D0) in the total component (D) contained in the resist composition according to the present embodiment is preferably 50% by mass or more, more preferably 70% by mass or more, and still more preferably 90% by mass or more, and the component (D) may consist of only the component (D0).

### <Other Components>

The resist composition according to the present embodiment may further contain other components in addition to the component (A) and the component (d) described above. Examples of the other components include a component (B), a component (E), a component (F), and a component (S), which are described below.

### Acid Generator Component (B)»

It is preferable that the resist composition according to the present embodiment further contain an acid generator component (B) that generates an acid upon exposure.

The component (B) is not particularly limited, and those which have been proposed so far as an acid generator for a chemical amplification-type resist composition in the related art can be used.

Examples of these acid generators are numerous and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators; iminosulfonate-based acid generators; and disulfonate-based acid generators.

Examples of the onium salt-based acid generator include a compound represented by General Formula (b-1) (hereinafter, also referred to as a “component (b-1)”), a compound represented by General Formula (b-2) (hereinafter, also referred to as a “component (b-2)”), and a compound represented by General Formula (b-3) (hereinafter, also referred to as a “component (b-3)”), which are described below.

Examples of the onium salt-based acid generator include a compound represented by General Formula (b-1) (hereinafter, also referred to as a “component (b-1)”), a compound represented by General Formula (b-2) (hereinafter, also referred to as a “component (b-2)”), and a compound represented by General Formula (b-3) (hereinafter, also referred to as a “component (b-3)”), which are described below.

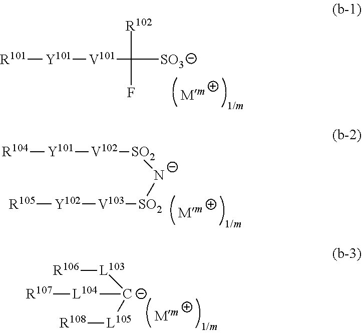

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. L¹⁰¹ and L¹⁰² each independently represent a single bond or an oxygen atom. L⁰ to L¹⁰⁵ each independently represent a single bond, —CO—, or —SO₂—. in represents an integer of 1 or more, and M′^{m+} represents an m-valent onium cation.]

### {Anion Moiety}

Anion in Component (b-1)

In General Formula (b-1), R¹⁰¹ represents a cyclic group which may have a substituent, a chain-like alkyl group which may have a substituent, or a chain-like alkenyl group which may have a substituent.

Cyclic group which may have substituent: The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group that has no aromaticity. The aliphatic hydrocarbon group may be saturated or unsaturated. In general, it is preferable that the aliphatic hydrocarbon group be saturated.

The aromatic hydrocarbon group as R¹⁰¹ is a hydrocarbon group having an aromatic ring. The aromatic hydrocarbon group preferably has 3 to 30 carbon atoms, more preferably has 5 to 30, still more preferably has 5 to 20, particularly preferably has 6 to 15, and most preferably has 6 to 10. However, the number of carbon atoms in a substituent is not included in the number of carbon atoms.

Specific examples of the aromatic ring of the aromatic hydrocarbon group as R¹⁰¹ include benzene, fluorene, naphthalene, anthracene, phenanthrene, biphenyl, and an aromatic heterocyclic ring in which some carbon atoms constituting any of these aromatic rings have been substituted with hetero atoms. Examples of the hetero atom in the aromatic heterocyclic rings include an oxygen atom, a sulfur atom, and a nitrogen atom.

Specific examples of the aromatic hydrocarbon group as R¹⁰¹ include a group in which one hydrogen atom has been removed from the aromatic ring (an aryl group such as a phenyl group or a naphthyl group) and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, a l-naphthylethyl group, or a 2-naphthylethyl group). The alkylene group (an alkyl chain in the arylalkyl group) preferably has 1 to 4 carbon atoms, more preferably has 1 or 2 carbon atoms, and particularly preferably has 1 carbon atom.

Examples of the cyclic aliphatic hydrocarbon group as R⁰¹ include an aliphatic hydrocarbon group having a ring in the structure thereof.

Examples of the aliphatic hydrocarbon group containing a ring in the structure thereof include an alicyclic hydrocarbon group (a group obtained by removing one hydrogen atom from an aliphatic hydrocarbon ring), a group in which the alicyclic hydrocarbon group is bonded to the terminal of a linear or branched aliphatic hydrocarbon group, and a group in which the alicyclic hydrocarbon group is interposed in a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group obtained by removing one or more hydrogen atoms from a monocycloalkane. The monocycloalkane preferably has 3 to 6 carbon atoms, and specific examples thereof include cyclopentane and cyclohexane. The polycyclic alicyclic hydrocarbon group is preferably a group obtained by removing one or more hydrogen atoms from a polycycloalkane, and the polycycloalkane preferably has 7 to 30 carbon atoms. Among the above, the polycycloalkane is more preferably a polycycloalkane having a bridged ring-based polycyclic skeleton, such as adamantane, norbornane, isobornane, tricyclodecane, or tetracyclododecane; or a polycycloalkane having a condensed ring-based polycyclic skeleton, such as a cyclic group having a steroid skeleton.

Among these examples, as the cyclic aliphatic hydrocarbon group as R¹⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is still more preferable, and an adamantyl group is particularly preferable.

The linear aliphatic hydrocarbon group which may be bonded to the alicyclic hydrocarbon group preferably has 1 to 10 carbon atoms, more preferably has 1 to 6 carbon atoms, still more preferably has 1 to 4 carbon atoms, and most preferably has 1 to 3 carbon atoms. As the linear aliphatic hydrocarbon group, a linear alkylene group is preferable, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group preferably has 2 to 10 carbon atoms, more preferably has 3 to 6 carbon atoms, still more preferably has 3 or 4 carbon atoms, and most preferably has 3 carbon atoms. As the branched aliphatic hydrocarbon group, a branched alkylene group is preferable, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Further, the cyclic hydrocarbon group as R¹⁰¹ may have a hetero atom such as a heterocyclic ring. Specific examples thereof include the lactone-containing cyclic groups represented by each of General Formulae (a2-r-1) to (a2-r-7), the —SO₂—containing cyclic groups represented by each of General Formulae (b5-r-1) to (b5-r-4), and other heterocyclic groups represented by each of Chemical Formulae (r-hr-1) to (r-hr-16). In the formulae, * represents a bonding site for bonding to Y¹⁰¹ in General Formula (b-1).

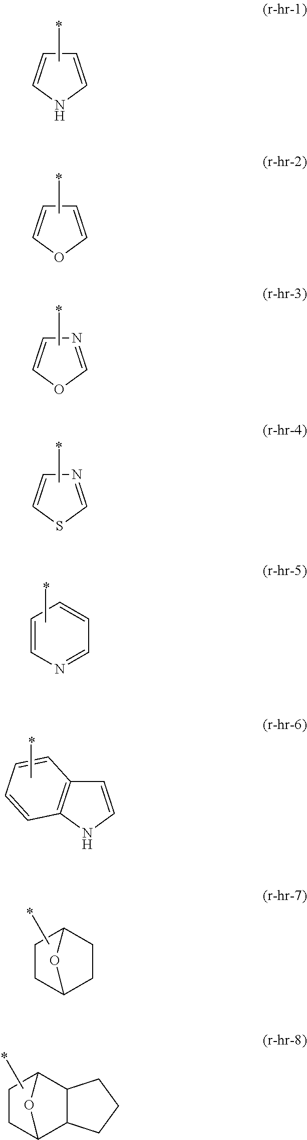

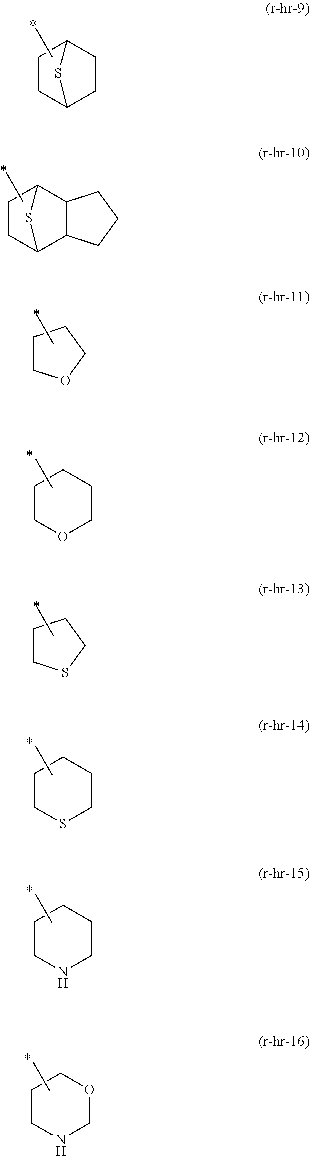

Examples of the substituent for the cyclic group as R¹⁰¹ include an alkyl group, an alkoxy group, a halogen atom, a halogenated alkyl group, a hydroxyl group, a carbonyl group, and a nitro group.

The alkyl group as the substituent is preferably an alkyl group having 1 to 5 carbon atoms, and a methyl group, an ethyl group, a propyl group, an n-butyl group, or a tert-butyl group is most preferable.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom for the substituent include a fluorine atom, a chlorine atom, a bromine atom, and an iodine atom, and a fluorine atom is preferable. Examples of the halogenated alkyl group as the substituent include a group obtained by substituting some or all hydrogen atoms in an alkyl group having 1 to 5 carbon atoms such as a methyl group, an ethyl group, a propyl group, an n-butyl group, or a tert-butyl group, with the above-described halogen atom.

The carbonyl group as the substituent is a group that substitutes a methylene group (—CH₂—) constituting the cyclic hydrocarbon group.

The cyclic hydrocarbon group as R¹⁰¹ may be a condensed cyclic group having a condensed ring in which an aliphatic hydrocarbon ring and an aromatic ring are condensed. Examples of the condensed ring include a condensed ring in which one or more aromatic rings are condensed with a polycycloalkane having a bridged ring-based polycyclic skeleton. Specific examples of the bridged ring-based polycycloalkane include bicycloalkanes such as bicyclo[2.2.1]heptane (norbornane) and bicyclo[2.2.2]octane. The condensed ring type is preferably a group containing a condensed ring, in which two or three aromatic rings are condensed with a bicycloalkane, and more preferably a group containing a condensed ring, in which two or three aromatic rings are condensed with bicyclo[2.2.2]octane. Specific examples of the condensed cyclic group as R¹⁰¹ include those represented by General Formulae (r-br-1) to (r-br-2) described above. Here, * in the formulae represents a bonding site for bonding to Y101 in General Formula (b-1).

Examples of the substituent which may be contained in the condensed cyclic group as R¹⁰¹ include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include those exemplified as the substituent of the cyclic group as R1.

Examples of the aromatic hydrocarbon group as the substituent of the condensed cyclic group include a group obtained by removing one hydrogen atom from the above-described aromatic ring (an aryl group; for example, a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and heterocyclic groups represented by each of General Formulae (r-hr-1) to (r-hr-6).

Examples of the alicyclic hydrocarbon group as the substituent of the condensed cyclic group include a group obtained by removing one hydrogen atom from a monocycloalkane such as cyclopentane or cyclohexane; a group obtained by removing one hydrogen atom from a polycycloalkane such as adamantane, norbornane, isobornane, tricyclodecane, or tetracyclododecane; the lactone-containing cyclic groups represented by each of General Formulae (a2-r-1) to (a2-r-7); the —SO₂—containing cyclic groups represented by each of General Formulae (b5-r-1) to (b5-r-4); and the heterocyclic groups represented by each of General Formulae (r-hr-7) to (r-hr-16).

Chain-like alkyl group which may have substituent:

The chain-like alkyl group as R″ ⁰ may be linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-like alkenyl group which may have substituent: The chain-like alkenyl group as R¹⁰¹ may be linear or branched, and the number of carbon atoms thereof is preferably in a range of 2 to 10, more preferably in a range of 2 to 5, still more preferably in a range of 2 to 4, and particularly preferably 3. Examples of the linear alkenyl group include a vinyl group, a propenyl group (an allyl group), and a butynyl group. Examples of the branched alkenyl group include a 1-methylvinyl group, a 2-methylvinyl group, a I-methylpropenyl group, and a 2-methylpropenyl group.

Among the above, the chain-like alkenyl group is preferably a linear alkenyl group, more preferably a vinyl group or a propenyl group, and particularly preferably a vinyl group.

Examples of the substituent for the chain-like alkyl group or alkenyl group as R¹⁰¹ include an alkoxy group, a halogen atom, a halogenated alkyl group, a hydroxyl group, a carbonyl group, a nitro group, an amino group, and a cyclic group as R¹⁰¹.

Among the examples, R¹⁰¹ is preferably a cyclic group which may have a substituent and more preferably a cyclic hydrocarbon group which may have a substituent. More specifically, the cyclic hydrocarbon group is preferably a group obtained by removing one or more hydrogen atoms from a phenyl group, a naphthyl group, a polycycloalkane; the lactone-containing cyclic groups represented by each of General Formulae (a2-r-1) to (a2-r-7); or the —SO₂—containing cyclic groups represented by each of General Formulae (b5-r-1) to (b5-r-4), more preferably a group obtained by removing one or more hydrogen atoms from a polycycloalkane, or the —SO₂—containing cyclic groups represented by each of General Formulae (b5-r-1) to (b5-r-4), and still more preferably an adamantyl group or the —SO₂—containing cyclic group represented by General Formula (b5-r-1).

In General Formula (b-1), Y101 represents a single bond or a divalent linking group having an oxygen atom.

In a case where Y¹⁰¹ represents a divalent linking group containing an oxygen atom, Y¹⁰¹ may contain an atom other than the oxygen atom. Examples of the atom other than the oxygen atom include a carbon atom, a hydrogen atom, a sulfur atom, and a nitrogen atom.

Examples of divalent linking groups containing an oxygen atom include non-hydrocarbon-based oxygen atom-containing linking groups such as an oxygen atom (an ether bond; —O—), an ester bond (—C(═O)—O—), an oxycarbonyl group (—O—C(═O)—), an amide bond (—C(═O)—NH—), a carbonyl group (—C(═O)—), or a carbonate bond (—O—C(═O)—O—); and combinations of the above-described non-hydrocarbon-based oxygen atom-containing linking groups with an alkylene group. Further, a sulfonyl group (—SO₂—) may be further linked to the combination. Examples of divalent linking groups containing an oxygen atom include linking groups represented by each of General Formulae (y-a1-1) to (y-a1-7) described above. Here, it is noted that the thing bonded to R¹⁰¹ in General Formula (b-1) is V′⁰¹ in General Formulae (y-a1-1) to (y-a1-7) described above.

Y¹⁰¹ is preferably a divalent linking group having an ester bond or a divalent linking group having an ether bond and more preferably a linking group represented by any of General Formulae (y-a1-1) to (y-a1-5).

In General Formula (b-1), V¹⁰ represents a single bond, an alkylene group, or a fluorinated alkylene group. It is preferable that the alkylene group and the fluorinated alkylene group as V¹⁰¹ have 1 to 4 carbon atoms. Examples of the fluorinated alkylene group as V¹⁰¹ include a group in which some or all hydrogen atoms in the alkylene group as V¹⁰¹ have been substituted with fluorine atoms. Among the above, V¹⁰¹ is preferably a single bond or a fluorinated alkylene group having 1 to 4 carbon atoms, and it is more preferably a linear fluorinated alkylene group having 1 to 4 carbon atoms.

In General Formula (b-1), R¹⁰² represents a fluorine atom or a fluorinated alkyl group having 1 to 5 carbon atoms. R¹⁰² is preferably a fluorine atom or a perfluoroalkyl group having 1 to 5 carbon atoms and more preferably a fluorine atom.

In a case where Y¹⁰¹ represents a single bond, specific examples of the anion moiety represented by General Formula (b-1) include fluorinated alkyl sulfonate anions such as a trifluoromethanesulfonate anion and a perfluorobutanesulfonate anion.

Anion in Component (b-2)

In General Formula (b-2), R¹⁰⁴ and R¹⁰⁵ each independently represent a cyclic group which may have a substituent, a chain-like alkyl group which may have a substituent, or a chain-like alkenyl group which may have a substituent, and examples thereof include the same groups as those for R^{10′} in General Formula (b-1). Here, R¹¹⁴ and R¹⁰⁵ may be bonded to each other to form a ring.

R¹⁰⁴ and R¹⁰⁵ are preferably a chain-like alkyl group which may have a substituent and more preferably a linear or branched alkyl group or a linear or branched fluorinated alkyl group.

The chain-like alkyl group preferably has 1 to 10 carbon atoms, more preferably has 1 to 7 carbon atoms, and still more preferably has 1 to 3 carbon atoms. It is preferable that the number of carbon atoms in the chain-like alkyl group as R¹⁰⁴ and R¹⁰⁵ decrease within the range of the number of carbon atoms from the viewpoint that the solubility in a solvent for a resist is also satisfactory. Further, in the chain-like alkyl group as R¹⁰⁴ and R¹⁰⁵, it is preferable that the number of hydrogen atoms substituted with fluorine atoms be as large as possible from the viewpoint that the acid strength increases and the transparency to high energy light or electron beams having a wavelength of 250 nm or less is improved. The proportion of fluorine atoms in the chain-like alkyl group, that is, the fluorination rate, is preferably in a range of 70% to 100% and more preferably in a range of 90% to 100%, and it is most preferable that the chain-like alkyl group be a perfluoroalkyl group in which all hydrogen atoms are substituted with fluorine atoms.

In General Formula (b-2), V¹⁰² and V¹⁰³ each independently represent a single bond, an alkylene group, or a fluorinated alkylene group, and examples thereof include the same groups as those for V¹⁰¹ in General Formula (b-1).

In General Formula (b-2), L¹⁰¹ and L¹⁰² each independently represent a single bond or an oxygen atom.

Anion in Component (b-3)

In General Formula (b-3), R¹⁰⁶ to R¹⁰⁸ each independently represent a cyclic group which may have a substituent, a chain-like alkyl group which may have a substituent, or a chain-like alkenyl group which may have a substituent, and examples thereof include the same groups as those for R¹⁰¹ in General Formula (b-1).

In General Formula (b-3), L¹⁰³ to L¹⁰⁵ each independently represent a single bond, —CO—, or —SO₂—.

Among the above, the anion moiety of the component (B) is preferably an anion of the component (b-1).

### {Cation Moiety}

In General Formulae (b-1), (b-2), and (b-3), M^{m+} represents an m-valent onium cation. Among them, a sulfonium cation and an iodonium cation are preferable. m represents an integer of 1 or more.

Preferred examples of the cation moiety ((M′^{m+})_{1/m}) include organic cations represented by each of General Formulae (ca-1) to (ca-3).

In the resist composition according to the present embodiment, the component (B) may be used alone or in a combination of two or more kinds thereof.

In a case where the resist composition contains the component (B), the content of the component (B) in the resist composition is preferably less than 40 parts by mass, more preferably in a range of 1 to 30 parts by mass, and still more preferably in a range of 1 to 25 parts by mass with respect to 100 parts by mass of the component (A).

In a case where the content of the component (B) is set to be in the preferred range described above, pattern formation can be sufficiently carried out. Further, in a case where each component of the resist composition is dissolved in an organic solvent, the above range is preferable since a homogeneous solution is easily obtained and the storage stability of the resist composition is improved.

«At Least One Compound (E) Selected from the Group Consisting of Organic Carboxylic Acid, Phosphorus Oxo Acid, and Derivatives Thereof»

For the purpose of preventing any deterioration in sensitivity and improving the resist pattern shape and the post-exposure temporal stability, the resist composition according to the present embodiment may contain, as an optional component, at least one compound (E) (hereinafter referred to as a “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid, and among them, salicylic acid is preferable.

Examples of the phosphorus oxo acid include phosphoric acid, phosphonic acid, and phosphinic acid. Among these, phosphonic acid is particularly preferable.

Examples of the phosphorus oxo acid derivative include an ester obtained by substituting a hydrogen atom in the above-described oxo acid with a hydrocarbon group. Examples of the hydrocarbon group include an alkyl group having 1 to 5 carbon atoms and an aryl group having 6 to 15 carbon atoms.

Examples of the phosphoric acid derivative include phosphoric acid esters such as di-n-butyl phosphate and diphenyl phosphate.

Examples of the phosphonic acid derivative include phosphonic acid esters such as dimethyl phosphonate, di-n-butyl phosphonate, phenylphosphonic acid, diphenyl phosphonate, and dibenzyl phosphonate.

Examples of the phosphinic acid derivative include phosphinic acid esters and phenylphosphinic acid.

In the resist composition according to the present embodiment, the component (E) may be used alone or in a combination of two or more kinds thereof.

In a case where the resist composition contains the component (E), the content of the component (E) is preferably in a range of 0.01 to 5 parts by mass and more preferably in a range of 0.05 to 3 parts by mass with respect to 100 parts by mass of the component (A). Within the above range, the lithography characteristics are further improved.

### «Fluorine Additive Component (F)»

The resist composition according to the present embodiment may further contain a fluorine additive component (hereinafter, referred to as a “component (F)”) as a hydrophobic resin. The component (F) is used to impart water repellency to the resist film and used as a resin different from the component (A), whereby the lithography characteristics can be improved.

As the component (F), it is possible to use, for example, a fluorine-containing polymeric compound described in Japanese Unexamined Patent Application, First Publication No. 2010-002870, Japanese Unexamined Patent Application, First Publication No. 2010-032994, Japanese Unexamined Patent Application, First Publication No. 2010-277043, Japanese Unexamined Patent Application, First Publication No. 2011-13569, or Japanese Unexamined Patent Application, First Publication No. 2011-128226.

Specific examples of the component (F) include polymers having a constitutional unit (f1) represented by General Formula (f1-1) shown below. This polymer is preferably a polymer (a homopolymer) consisting only of a constitutional unit (f1) represented by General Formula (f1-1); a copolymer of the constitutional unit (f1) and the constitutional unit (a1); a copolymer of the constitutional unit (f1), a constitutional unit derived from acrylic acid or methacrylic acid, and the constitutional unit (a1), and more preferably a copolymer of the constitutional unit (f1) and the constitutional unit (a1). The constitutional unit (a1) to be copolymerized with the constitutional unit (f1) is preferably a constitutional unit derived from 1-ethyl-1-cyclooctyl(meth)acrylate or a constitutional unit derived from 1-methyl-1-adamantyl (meth)acrylate, and more preferably a constitutional unit derived from 1-ethyl-I-cyclooctyl(meth)acrylate.

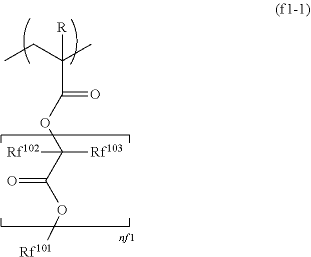

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf⁴ represents an integer in a range of 0 to 5, and Rf¹⁰¹ represents an organic group having a fluorine atom.]

In General Formula (f1-1), R bonded to the carbon atom at the α-position has the same definition as described above. R is preferably a hydrogen atom or a methyl group.

In General Formula (f1-y), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atom as R. Among the examples, a methyl group or an ethyl group is preferable. Specific examples of the halogenated alkyl group having l to 5 carbon atoms as Rf¹⁰² and Rf¹⁰³ include groups in which some or all hydrogen atoms of an alkyl group having to 5 carbon atoms have been substituted with halogen atoms. The halogen atom is preferably a fluorine atom. Among these, Rf¹⁰² and Rf¹⁰³ are preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In General Formula (f1-1), nf¹ represents an integer in a range of 0 to 5, preferably an integer in a range of 0 to 3, and more preferably 1 or 2.

In General Formula (f1-1), Rf¹⁰¹ represents an organic group having a fluorine atom and preferably a hydrocarbon group having a fluorine atom.

The hydrocarbon group containing a fluorine atom may be linear, branched, or cyclic, and it preferably has 1 to 20 carbon atoms, more preferably has 1 to 15 carbon atoms, and particularly preferably has 1 to 10 carbon atoms.

In addition, in the hydrocarbon group containing a fluorine atom, 25% or more of the hydrogen atoms in the hydrocarbon group are preferably fluorinated, more preferably 50% or more are fluorinated, and particularly preferably 60% or more are fluorinated to increase the hydrophobicity of the resist film during immersion exposure.

Among the examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight average molecular weight (Mw) (in terms of the polystyrene equivalent value determined by gel permeation chromatography) of the component (F) is preferably in a range of 1,000 to 50,000, more preferably in a range of 5,000 to 40,000, and most preferably in a range of 10,000 to 30,000. In a case where the weight average molecular weight is equal to or smaller than the upper limit value of this range, the resist composition exhibits sufficient solubility in a resist solvent to be used as a resist. On the other hand, in a case where the weight average molecular weight is equal to or larger than the lower limit value of this range, the water repellency of the resist film is excellent.

Further, the polydispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or in a combination of two or more kinds thereof. In a case where the resist composition contains the component (F), the content of the component (F) in the resist composition is preferably in a range of 0.5 to 10 parts by mass and more preferably in a range of 1 to 10 parts by mass with respect to 100 parts by mass of the component (A).

### «Organic Solvent Component (S)»

The resist composition according to the present embodiment may be produced by dissolving the resist materials in an organic solvent component (hereinafter, referred to as a “component (S)”).

The component (S) may be any organic solvent which can dissolve each of the components to be used to obtain a homogeneous solution, and an optional organic solvent appropriately selected from those which are conventionally known in the related art as solvents for a chemical amplification-type resist composition can be used.

Examples of the component (S) include lactones such as γ-butyrolactone; ketones such as acetone, methyl ethyl ketone, cyclohexanone, methyl-n-pentyl ketone, methyl isopentyl ketone, and 2-heptanone; polyhydric alcohols, such as ethylene glycol, diethylene glycol, propylene glycol and dipropylene glycol; compounds having an ester bond, such as ethylene glycol monoacetate, diethylene glycol monoacetate, propylene glycol monoacetate, and dipropylene glycol monoacetate; polyhydric alcohol derivatives including compounds having an ether bond, such as a monoalkyl ether (such as monomethyl ether, monoethyl ether, monopropyl ether or monobutyl ether) or monophenyl ether of any of these polyhydric alcohols or compounds having an ester bond [among these, propylene glycol monomethyl ether acetate (PGMEA) and propylene glycol monomethyl ether (PGME) are preferable]; cyclic ethers such as dioxane; esters such as methyl lactate, ethyl lactate (EL), methyl acetate, ethyl acetate, butyl acetate, methyl pyruvate, ethyl pyruvate, methyl methoxypropionate, and ethyl ethoxypropionate; aromatic organic solvents such as anisole, ethylbenzyl ether, cresylmethyl ether, diphenyl ether, dibenzyl ether, phenetole, butylphenyl ether, ethyl benzene, diethyl benzene, pentyl benzene, isopropyl benzene, toluene, xylene, cymene and mesitylene; and dimethylsulfoxide (DMSO).

In the resist composition according to the present embodiment, the component (S) may be used alone or as a mixed solvent of two or more kinds thereof. Among these, PGMEA, PGME, γ-butyrolactone, EL, and cyclohexanone are preferable.

Further, a mixed solvent obtained by mixing PGMEA with a polar solvent is also preferable as the component (S). The blending ratio (mass ratio) of the mixed solvent can be appropriately determined, taking into consideration the compatibility of the PGMEA with the polar solvent, but is preferably in a range of 1:9 to 9:1 and more preferably in a range of 2:8 to 8:2.

More specifically, in a case where EL or cyclohexanone is blended as the polar solvent, the PGMEA:EL or cyclohexanone mass ratio is preferably in a range of 1:9 to 9:1 and more preferably in a range of 2:8 to 8:2. Alternatively, in a case where PGME is blended as the polar solvent, the PGMEA:PGME mass ratio is preferably in a range of 1:9 to 9:1, more preferably in a range of 2:8 to 8:2, and still more preferably in a range of 3:7 to 7:3. Furthermore, a mixed solvent of PGMEA, PGME, and cyclohexanone is also preferable.

Further, the component (S) is also preferably a mixed solvent of at least one selected from PGMEA and EL and γ-butyrolactone. In this case, as the mixing ratio, the mass ratio of the former to the latter is preferably in a range of 70:30 to 95:5.

The amount of the component (S) to be used is not particularly limited and is appropriately set, depending on a thickness of a film to be coated, to a concentration at which the component (S) can be applied onto a substrate or the like. Generally, the component (S) is used such that the solid content concentration of the resist composition is in a range of 0.1% to 20% by mass and preferably in a range of 0.2% to 15% by mass.

As desired, other miscible additives can also be added to the resist composition according to the present embodiment. For example, for improving the performance of the resist film, an additive resin, a dissolution inhibitor, a plasticizer, a stabilizer, a colorant, a halation prevention agent, and a dye can be appropriately contained therein.

After dissolving the resist material in the component (S), the resist composition according to the present embodiment may be subjected to removal of impurities and the like by using a porous polyimide membrane, a porous polyamideimide membrane, or the like. For example, the resist composition may be filtered using a filter consisting of a porous polyimide membrane, a filter consisting of a porous polyamideimide membrane, or a filter consisting of a porous polyimide membrane and a porous polyamideimide membrane. Examples of the porous polyimide membrane and the porous polyamideimide membrane include those described in Japanese Unexamined Patent Application, First Publication No. 2016-155121.

The resist composition according to the present embodiment described above contains the compound (D0) (the component (D0)) represented by General Formula (d0).

The component (D0) controls the diffusion of the acid generated from the component (B) and the like in unexposed portions, whereas it generates an acid and acts as an acid generator in exposed portions.

The anion moiety of the component (D0) has a specific bulky structure (a condensed cyclic group containing a condensed ring containing one or more aromatic rings). This makes it possible to suitably control the diffusion of the acid generated from the component (D0) in the exposed portions. In addition, the improvement of the hydrophobicity of the component (D0) enhances the uniformity of the component (D0) in the resist film. As a result, for example, the acid generated from the component (B) is evenly trapped by the component (D0) at the boundary between exposed portions and unexposed portions of the resist film.

In addition, the bromine atom or iodine atom contained in the anion moiety of the component (D0) has a high absorption efficiency of extreme ultraviolet rays (EUV) or an electron beam (EB). As a result, the sensitivity to EUV or EB in exposed portions can be further improved as compared with a case where the acid diffusion control agent in the related art, which has no bromine atom or iodine atom, is used.

As a result, it is presumed that the resist composition containing the component (D0) according to the present embodiment makes it possible to achieve high sensitivity and makes it possible to form a resist pattern having good CDU.

### (Resist Pattern Forming Method)

A resist pattern forming method according to the second aspect according to the present invention is a method including a step of forming a resist film on a support using the resist composition according to the first aspect of the present invention described above, a step of exposing the resist film, and a step of developing the exposed resist film to form a resist pattern.

Examples of one embodiment of such a resist pattern forming method include a resist pattern forming method carried out as described below.

First, the resist composition of the above-described embodiment is applied onto a support with a spinner or the like, and a baking (post-apply bake (PAB)) treatment is carried out, for example, at a temperature condition in a range of 80° C. to 150° C. for 40 to 120 seconds, preferably for 60 to 90 seconds to form a resist film.

Following the selective exposure carried out on the resist film by, for example, exposure through a mask (mask pattern) having a predetermined pattern formed on the mask by using an exposure apparatus such as an electron beam lithography apparatus or an ArF exposure apparatus, or direct irradiation of the resist film for drawing with an electron beam without using a mask pattern, baking treatment (post-exposure bake (PEB)) is carried out, for example, under a temperature condition in a range of 80° C. to 150° C. for 40 to 120 seconds and preferably 60 to 90 seconds.

Next, the resist film is subjected to a developing treatment. The developing treatment is carried out using an alkali developing solution in the case of an alkali developing process, and a developing solution containing an organic solvent (organic developing solution) in the case of a solvent developing process.

After the developing treatment, it is preferable to conduct a rinse treatment. As the rinse treatment, water rinsing using pure water is preferable in the case of an alkali developing process, and rinsing using a rinse liquid containing an organic solvent is preferable in the case of a solvent developing process.

In the case of a solvent developing process, after the developing treatment or the rinse treatment, the developing solution or the rinse liquid remaining on the pattern can be removed by a treatment using a supercritical fluid.

After the developing treatment or the rinse treatment, drying is conducted. As desired, baking treatment (post-baking) can be carried out following the developing treatment.

In this manner, a resist pattern can be formed.

The support is not specifically limited and a known support in the related art can be used. For example, substrates for electronic components, and such substrates having a predetermined wiring pattern formed thereon can be used. Specific examples of the material of the substrate include metals such as a silicon wafer, copper, chromium, iron and aluminum; and glass. Suitable materials for the wiring pattern include copper, aluminum, nickel, and gold.

Further, as the support, any support having the above-described substrate on which an inorganic and/or organic film is provided may be used. Examples of the inorganic film include an inorganic antireflection film (an inorganic BARC). Examples of the organic film include an organic antireflection film (organic BARC) and an organic film such as a lower-layer organic film used in a multilayer resist method.

Here, the multilayer resist method is a method in which at least one layer of an organic film (lower-layer organic film) and at least one layer of a resist film (upper-layer resist film) are provided on a substrate, and a resist pattern formed on the upper-layer resist film is used as a mask to conduct patterning of the lower-layer organic film. This method is considered to enable a pattern to be formed with a high aspect ratio. More specifically, in the multilayer resist method, a desired thickness can be ensured by the lower-layer organic film, and as a result, the thickness of the resist film can be reduced, and an extremely fine pattern with a high aspect ratio can be formed.

The multilayer resist method is basically classified into a method in which a double-layer structure consisting of an upper-layer resist film and a lower-layer organic film is formed (double-layer resist method), and a method in which a multilayer structure having at least three layers consisting of an upper-layer resist film, a lower-layer organic film and at least one intermediate layer (thin metal film or the like) provided between the upper-layer resist film and the lower-layer organic film (triple-layer resist method) is formed.

The wavelength to be used for exposure is not particularly limited and the exposure can be conducted using radiation such as an ArF excimer laser, a KrF excimer laser, an F₂ excimer laser, extreme ultraviolet rays (EUV), vacuum ultraviolet rays (VUV), an electron beam (EB), X-rays, and soft X-rays. The resist composition is highly useful for a KrF excimer laser, an ArF excimer laser, EB, or EUV, more useful for an ArF excimer laser, EB, or EUV, and particularly useful for EB or EUV. That is, the resist pattern forming method according to the present embodiment is a useful method particularly in a case where the step of exposing the resist film includes an operation of exposing the resist film to extreme ultraviolet rays (EUV) ray or an electron beam (EB).

The exposure method of the resist film can be general exposure (dry exposure) carried out in air or an inert gas such as nitrogen, or liquid immersion lithography; however, liquid immersion lithography is more preferable.

Liquid immersion lithography is an exposure method in which the region between the resist film and the lens at the lowermost position of the exposure apparatus is pre-filled with a solvent (liquid immersion medium) that has a larger refractive index than air, and the exposure (immersion exposure) is carried out in this state. As the liquid immersion medium, a solvent that exhibits a refractive index larger than the refractive index of air but smaller than the refractive index of the resist film to be exposed is preferable. The refractive index of the solvent is not particularly limited as long as it satisfies the above-described requirements.

Examples of the solvent which exhibits a refractive index that is larger than the refractive index of air but smaller than the refractive index of the resist film include water, fluorine-based inert liquids, silicon-based solvents, and hydrocarbon-based solvents.

Specific examples of the fluorine-based inert liquid include a liquid containing a fluorine-based compound such as C₃HCl₂F₅, C₄F₉OCH₃, C₄F₉OC₂H₅, or C₅H₃F₇ as a main component, a liquid with a boiling point of 70° C. to 180° C. is preferable, and a liquid with a boiling point of 80° C. to 160° C. is more preferable. A fluorine-based inert liquid having a boiling point in the above-described range is advantageous in that removing the medium used in the liquid immersion after the exposure can be preferably carried out by a simple method.

The fluorine-based inert liquid is particularly preferably a perfluoroalkyl compound in which all of the hydrogen atoms of the alkyl group are substituted with fluorine atoms. Specific examples of these perfluoroalkyl compounds include perfluoroalkyl ether compounds and perfluoroalkyl amine compounds.

Specifically, an example of a suitable perfluoroalkyl ether compound is perfluoro(2-butyl-tetrahydrofuran) (boiling point of 102° C.), and an example of a suitable perfluoroalkyl amine compound is perfluorotributyl amine (boiling point of 174° C.).

As the liquid immersion medium, water is preferable in terms of cost, safety, environment, and versatility.

Examples of the alkali developing solution used for a developing treatment in an alkali developing process include a 0.1% to 10% by mass aqueous solution of tetramethylammonium hydroxide (TMAH).

As the organic solvent contained in the organic developing solution, which is used for a developing treatment in a solvent developing process, any one of the conventionally known organic solvents capable of dissolving the component (A) (component (A) prior to exposure) can be appropriately selected from the conventionally known organic solvents. Specific examples of the organic solvent include polar solvents such as a ketone-based solvent, an ester-based solvent, an alcohol-based solvent, a nitrile-based solvent, an amide-based solvent, an ether-based solvent, and a hydrocarbon-based solvent.

A ketone-based solvent is an organic solvent containing C—C(═O)—C in the structure thereof. An ester-based solvent is an organic solvent containing C—C(═O)—O—C in the structure thereof. An alcohol-based solvent is an organic solvent containing an alcoholic hydroxyl group in the structure thereof. The term “alcoholic hydroxyl group” indicates a hydroxyl group bonded to a carbon atom of an aliphatic hydrocarbon group. A nitrile-based solvent is an organic solvent containing a nitrile group in the structure thereof. An amide-based solvent is an organic solvent containing an amide group in the structure thereof. An ether-based solvent is an organic solvent containing C—O—C in the structure thereof.

Some organic solvents have a plurality of the functional groups which characterize the above-described solvents in the structure thereof. In such a case, the organic solvent can be classified as any type of solvent having a characteristic functional group. For example, diethylene glycol monomethyl ether can be classified as an alcohol-based solvent or an ether-based solvent.

A hydrocarbon-based solvent consists of a hydrocarbon which may be halogenated and does not have any substituent other than a halogen atom. The halogen atom is preferably a fluorine atom.

Among the above, the organic solvent contained in the organic developing solution is preferably a polar solvent and preferably a ketone-based solvent, an ester-based solvent, or a nitrile-based solvent.

Examples of the ketone-based solvent include 1-octanone, 2-octanone, 1-nonanone, 2-nonanone, acetone, 4-heptanone, 1-hexanone, 2-hexanone, diisobutyl ketone, cyclohexanone, methylcyclohexanone, phenylacetone, methyl ethyl ketone, methyl isobutyl ketone, acetylacetone, acetonylacetone, ionone, diacetonyl alcohol, acetylcarbinol, acetophenone, methyl naphthyl ketone, isophorone, propylene carbonate, γ-butyrolactone, and methylamyl ketone (2-heptanone). Among these examples, the ketone-based solvent is preferably methylamyl ketone (2-heptanone).

Examples of the ester-based solvent include methyl acetate, butyl acetate, ethyl acetate, isopropyl acetate, amyl acetate, isoamyl acetate, ethyl methoxyacetate, ethyl ethoxyacetate, ethylene glycol monoethyl ether acetate, ethylene glycol monopropyl ether acetate, ethylene glycol monobutyl ether acetate, ethylene glycol monophenyl ether acetate, diethylene glycol monomethyl ether acetate, diethylene glycol monopropyl ether acetate, diethylene glycol monophenyl ether acetate, diethylene glycol monobutyl ether acetate, diethylene glycol monoethyl ether acetate, 2-methoxybutyl acetate, 3-methoxybutyl acetate, 4-methoxybutyl acetate, 3-methyl-3-methoxybutyl acetate, 3-ethyl-3-methoxybutyl acetate, propylene glycol monomethyl ether acetate, propylene glycol monoethyl ether acetate, propylene glycol monopropyl ether acetate, 2-ethoxybutyl acetate, 4-ethoxybutyl acetate, 4-propoxybutyl acetate, 2-methoxypentyl acetate, 3-methoxypentyl acetate, 4-methoxypentyl acetate, 2-methyl-3-methoxypentyl acetate, 3-methyl-3-methoxypentyl acetate, 3-methyl-4-methoxypentyl acetate, 4-methyl-4-methoxypentyl acetate, propylene glycol diacetate, methyl formate, ethyl formate, butyl formate, propyl formate, ethyl lactate, butyl lactate, propyl lactate, ethyl carbonate, propyl carbonate, butyl carbonate, methyl pyruvate, ethyl pyruvate, propyl pyruvate, butyl pyruvate, methyl acetoacetate, ethyl acetoacetate, methyl propionate, ethyl propionate, propyl propionate, isopropyl propionate, methyl 2-hydroxypropionate, ethyl 2-hydroxypropionate, methyl-3-methoxypropionate, ethyl-3-methoxypropionate, ethyl-3-ethoxypropionate, and propyl-3-methoxypropionate. Among these, the ester-based solvent is preferably butyl acetate.

Examples of the nitrile-based solvent include acetonitrile, propionitrile, valeronitrile, and butyronitrile.

As desired, the organic developing solution may have a conventionally known additive blended. Examples of the additive include surfactants. The surfactant is not particularly limited, and for example, an ionic or non-ionic fluorine-based and/or a silicon-based surfactant can be used. As the surfactant, a non-ionic surfactant is preferable, and a non-ionic fluorine-based surfactant or a non-ionic silicon-based surfactant is more preferable.

In a case where a surfactant is blended, the blending amount thereof is typically in a range of 0.001% to 5% by mass, preferably in a range of 0.005% to 2% by mass, and more preferably in a range of 0.01% to 0.5% by mass with respect to the total amount of the organic developing solution.

The developing treatment can be carried out by a conventionally known developing method. Examples thereof include a method in which the support is immersed in the developing solution for a predetermined time (a dip method), a method in which the developing solution is cast upon the surface of the support by surface tension and maintained for a predetermined time (a puddle method), a method in which the developing solution is sprayed onto the surface of the support (spray method), and a method in which a developing solution is continuously ejected from a developing solution ejecting nozzle and applied onto a support which is scanned at a constant rate while being rotated at a constant rate (dynamic dispense method).

As the organic solvent contained in the rinse liquid used in the rinse treatment after the developing treatment in a case of a solvent developing process, for example, an organic solvent that does not easily dissolve the resist pattern can be appropriately selected and used, among the organic solvents mentioned as organic solvents that are used for the organic developing solution. In general, at least one kind of solvent selected from a hydrocarbon-based solvent, a ketone-based solvent, an ester-based solvent, an alcohol-based solvent, an amide-based solvent, and an ether-based solvent is used. Among these, at least one kind of solvent selected from a hydrocarbon-based solvent, a ketone-based solvent, an ester-based solvent, an alcohol-based solvent, and an amide-based solvent is preferable, at least one kind of solvent selected from an alcohol-based solvent and an ester-based solvent is more preferable, and an alcohol-based solvent is particularly preferable.

The alcohol-based solvent used for the rinse liquid is preferably a monohydric alcohol of 6 to 8 carbon atoms, and the monohydric alcohol may be linear, branched, or cyclic. Specific examples thereof include 1-hexanol, 1-heptanol, 1-octanol, 2-hexanol, 2-heptanol, 2-octanol, 3-hexanol, 3-heptanol, 3-octanol, 4-octanol, and benzyl alcohol. Among these, 1-hexanol, 2-heptanol, and 2-hexanol are preferable, and 1-hexanol and 2-hexanol are more preferable.

As the organic solvent, one kind of solvent may be used alone, or two or more kinds of solvents may be used in combination. Further, an organic solvent other than the above-described examples or water may be mixed therewith. However, in consideration of the development characteristics, the amount of water to be blended in the rinse liquid is preferably 30% by mass or less, more preferably 10% by mass or less, still more preferably 5% by mass or less, and particularly preferably 3% by mass or less with respect to the total amount of the rinse liquid.

A conventionally known additive can be blended with the rinse liquid as necessary. Examples of the additive include surfactants. Examples of the surfactant include the same ones as those described above, and the surfactant is preferably a non-ionic surfactant and more preferably a non-ionic fluorine-based surfactant or a non-ionic silicon-based surfactant.

In a case where a surfactant is blended, the blending amount thereof is typically in a range of 0.001% to 5% by mass, preferably in a range of 0.005% to 2% by mass, and more preferably in a range of 0.01% to 0.5% by mass with respect to the total amount of the rinse liquid.

The rinse treatment using a rinse liquid (washing treatment) can be carried out by a conventionally known rinse method. Examples of the rinse treatment method include a method in which the rinse liquid is continuously ejected and applied onto the support while rotating it at a constant rate (rotational coating method), a method in which the support is immersed in the rinse liquid for a predetermined time (dip method), and a method in which the rinse liquid is sprayed onto the surface of the support (spray method).

According to the resist pattern forming method according to the present embodiment described above, since the resist composition described above is used, it is possible to achieve high sensitivity and form a resist pattern having good CDU.

Various materials that are used in the resist composition according to the above-described embodiment and the method for forming a pattern according to the above-described embodiment (for example, a resist solvent, a developing solution, a rinse liquid, a composition for forming an antireflection film, and a composition for forming a top coat) preferably do not contain impurities such as a metal, a metal salt containing a halogen, an acid, an alkali, and a component containing a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, and particularly preferably 10 ppt or less, and it is most preferable that the impurities be substantially absent (below the detection limit of the measuring device).

### (Compound)

The compound according to the third aspect of the present invention is a compound represented by General Formula (d0).

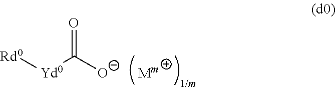

[In the formula, Rd⁰ represents a condensed cyclic group in which an aromatic ring and an alicyclic ring are condensed. The alicyclic ring in the condensed cyclic group has a substituent, and at least one of the substituents includes a hydrocarbon group having a bromine atom or a hydrocarbon group having an iodine atom. Yd⁰ represents a divalent linking group or a single bond. However, Yd⁰ is bonded to the alicyclic ring in the condensed cyclic group. M^{m+} represents an m-valent organic cation. m represents an integer of 1 or more.]

The compound represented by General Formula (d0) is the same as the component (D0) in the resist composition according to the first aspect of the present invention described above.

[Method of Producing Compound Represented by General Formula (d0)]

The component (D0) can be produced by using a known method.

As a specific method of producing the component (D0), a method of producing a compound represented by General Formula (d′0), which is an example of the component (D0), is described below.

First, a compound X1 represented by General Formula (X-1) is reacted with a compound (A1c-1) represented by General Formula (A1c-1), which has a desired hydrocarbon group having a bromine atom or has a desired hydrocarbon group (Rbi) having an iodine atom, to obtain a compound D0 pre represented by General Formula (D0 pre) (a first step).

Next, the compound (D0 pre) and a compound (S-1) represented by General Formula (S-1) are subjected to a salt exchange reaction in the presence of a base, which makes it possible to obtain a compound represented by General Formula (d′0), which is an example of the component (D0) (a second step).

It is noted that, in the reaction formula described below, “RbiO—C═O—Rd⁰⁰” is an example of “Rd⁰” in General Formula (d0) although it is denoted as “RbiO—C═O—Rd⁰⁰” for convenience.

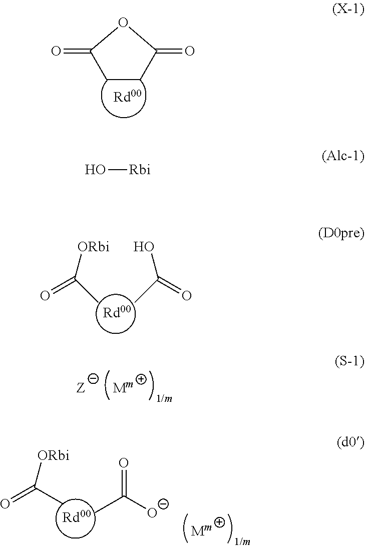

[In the formula, Rd⁰⁰ represents a condensed cyclic group in which an aromatic ring and an alicyclic ring are condensed. Rbi represents a hydrocarbon group having a bromine atom or a hydrocarbon group having an iodine atom. Z⁻ represents a halogen ion. (M^{m+})_{1/m} represents an m-valent organic cation. m represents an integer of 1 or more.]

### First Step:

The first step is a step of dissolving, for example, the compound (X-1) and the compound (A1c-1) in an organic solvent (THF or the like) and reacting them in the presence of a base to obtain the compound (D0 pre).

Specific examples of the base include sodium hydride, K₂CO₃, Cs₂CO₃, lithium diisopropylamide (LDA), triethyl amine, and 4-dimethylaminopyridine.

The reaction temperature is, for example, in a range of 0° C. to 50° C., and the reaction time is, for example, 10 minutes or more and 24 hours or less.

In the formula, Rd⁰ represents a condensed cyclic group in which an aromatic ring and an alicyclic ring are condensed, and it is the same as the condensed cyclic group as Rd⁰ in General Formula (d0), in which an aromatic ring and an alicyclic ring are condensed.

### Second Step:

The second step is a step of reacting, for example, the compound (D0 pre) with the compound (S-1) for salt exchange in the presence of a solvent such as water, dichloromethane, acetonitrile, or chloroform, and a base to obtain a compound represented by General Formula (d′0), which is an example of the component (D0).

Specific examples of the base include tetramethylammonium hydroxide (TMAH), sodium hydride, K₂CO₃, Cs₂CO₃, lithium diisopropylamide (LDA), triethyl amine, and 4-dimethylaminopyridine.

Specific examples of Z⁻ in the above formula include a bromine ion and a chloride ion.

The reaction temperature is, for example, in a range of 0° C. to 100° C., and the reaction time is, for example, 10 minutes or more and 24 hours or less.

In the formula, (M^{m+})_{1/m} is the same as (M^{m+})_{1/m} in General Formula (d0).

After the salt exchange reaction is completed, the compound in the reaction solution may be isolated and purified. A known method in the related art can be used for isolation and purification, and for example, concentration, solvent extraction, distillation, crystallization, recrystallization, or chromatography can be appropriately combined and used.

The structure of the compound obtained as described above can be identified by general organic analysis methods such as ¹H-nuclear magnetic resonance (NMR) spectroscopy, ³C-NMR spectroscopy, ¹⁹F-NMR spectroscopy, infrared (IR) absorption spectroscopy, mass spectrometry (MS), elemental analysis, and X-ray crystal diffraction.

In the method of producing the component (D0), a step of reacting the compound (D0 pre) with a hydroxy acid to obtain a compound represented by General Formula (D0 pre), which is different from the compound (D0 pre), may be provided between the first step and the second step.

Specific examples of the hydroxy acid include a compound represented by Chemical Formula (K-1), a compound represented by Chemical Formula (K-2), and a compound represented by Chemical Formula (K-3).

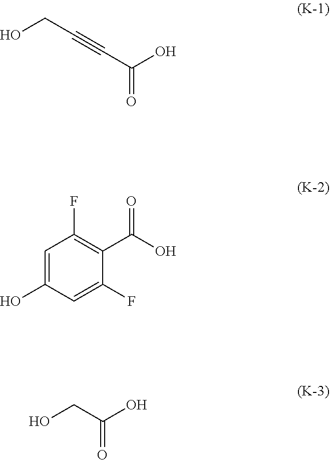 [SMILES: O=C(O)C#CCO ; O=C(O)c1c(F)cc(O)cc1F ; O=C(O)CO]

Further, the method of producing the component (D0) may include a step of reacting the compound (D0 pre) obtained in the first step with a diol such as ethylene glycol to obtain an intermediate and reacting the obtained intermediate with a dicarboxylic acid such as oxalic acid to obtain a compound represented by General Formula (D0 pre) which is different from the compound (D0 pre).

As the raw material that is used in each step, a commercially available raw material may be used, or a synthetic material may be used.

For example, in the case of synthesizing the compound (X-1), the compound (X-1) can be obtained by carrying out a Diels-Alder reaction between an aromatic compound (for example, anthracene) and an alkene (for example, maleic acid anhydride).

The compound according to the third aspect of the present invention described above is a compound useful as an acid diffusion control agent in the resist composition according to the first aspect of the present invention described above.

### (Acid Diffusion Control Agent)

An acid diffusion control agent according to a fourth aspect of the present invention contains the compound according to the third aspect described above.

Such an acid diffusion control agent is useful as an acid diffusion control agent for a chemical amplification-type resist composition.

Since the compound according to the third aspect described above has a carboxylate anion in the anion moiety, it generates upon exposure a relatively weak acid as compared with a fluorinated alkyl sulfonate anion or the like contained in the anion moiety of the acid generator that is generally used in the chemical amplification-type resist composition.

In a case where such an acid diffusion control agent is used in a chemical amplification-type resist composition, sensitivity and CDU are further improved in the resist pattern formation. In a case where such an acid diffusion control agent is used, sensitivity and CDU are further improved, particularly in the resist pattern formation using an EB or EUV light source.

### EXAMPLES

Hereinafter, the present invention will be described in more detail based on Examples, but the present invention is not limited to these Examples.

### <Production of Compound (X-1)>

### Production Example 1-1

Anthracene (20.0 g, 112.2 mmol), maleic acid anhydride (16.6 g, 168.3 mmol), aluminum chloride (1.50 g, 11.2 mmol), and toluene (200 g) were charged into a 300 mL three-necked flask and reacted at 80° C. for 4 hours with stirring. After cooling, ultrapure water (155 g) was added thereto, and after carrying out stirring for 30 minutes, the precipitated solid was filtered. The filtrate was dissolved in a mixed solvent of THF (93 g) and dichloromethane (680 g), washed 3 times with ultrapure water (155 g), and then the organic layer was concentrated using a rotary evaporator. The concentrate was recrystallized with ethyl acetate to obtain a compound (X-1-1).

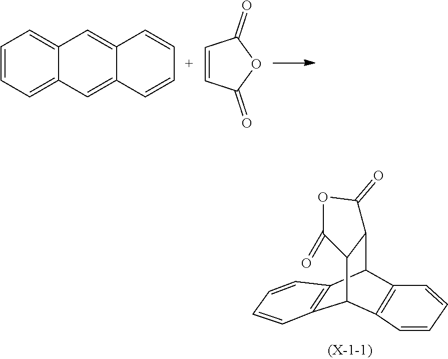

<Production of Compound (D0 pre)>

### Production Example 2-1

Sodium hydride (60% in oil) (4.3 g, 106.0 mmol) and dewatered THF (73.2 g) were charged into a 300 mL three-necked flask and cooled to 10° C. or lower. 2,4,6-triiodophenol (25.0 g, 53.0 mmol) was added to the suspension, the resultant mixture was stirred for 30 minutes as it was, and then the compound (X-1-1) (14.6 g, 53.0 mmol) was added thereto, and the temperature was returned to room temperature (25° C.). After 6 hours, the reaction solution was added dropwise to 5% hydrochloric acid (91.1 g, 127.2 mmol) which had been cooled to 10° C. or lower. After carrying out stirring for 1 hour, liquid separation was carried out, and the organic layer was concentrated using a rotary evaporator. Dichloromethane (79 g) was added to the concentrate, stirring was followed at room temperature (25° C.) for 2 hours, and the precipitated solid was filtered. Acetonitrile (337 g) was added to the obtained solid to dissolve the solid at 60° C., and then the temperature was returned to room temperature (25° C.). Ultrapure water (337 g) was added thereto, and then cooling was carried out to 10° C. or lower. After 2 hours, the precipitated solid was filtered to obtain a compound (D0 pre-01).

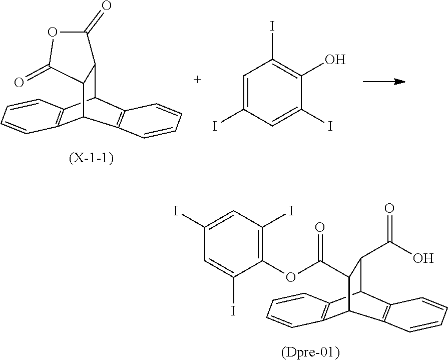

### Production Example 2-2

The compound (D0 pre-0l) (10.9 g, 14.6 mmol), the compound (K-1) (1.6 g, 16.1 mmol), and dichloromethane (85 g) were charged into a 300 m L three-necked flask and stirred and dissolved at room temperature (25° C.). Next, diisopropylcarbodiimide (2.1 g, 16.1 mmol) and dimethylaminopyridine (0.028 g, 0.2 mmol) were added thereto and reacted at room temperature for 5 hours. The reaction solution was filtered, and the filtrate was concentrated using a rotary evaporator. The concentrate was dissolved in acetonitrile (15 g) and subsequently added dropwise into MTBE (90 g), and the precipitated solid was filtered. The filtrate was dissolved in acetonitrile (15 g) and added dropwise into MTBE (90 g), and the precipitated solid was filtered. After repeating this operation twice, the filtrate was dried under reduced pressure to obtain a compound (Dpre-02).

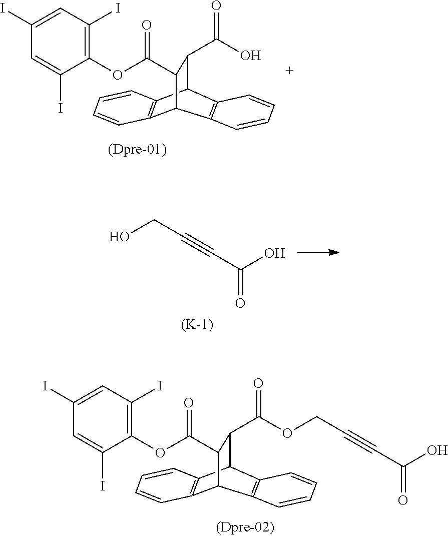 [SMILES: O=C(O)[C@H]1C2c3ccccc3C(c3ccccc32)[C@@H]1C(=O)Oc1c(I)cc(I)cc1I ; O=C(O)C#CCO ; "(K-1)" ; O=C(O)C#CCOC(=O)[C@H]1C2c3ccccc3C(c3ccccc32)[C@@H]1C(=O)Oc1c(I)cc(I)cc1I]

### Production Example 2-3

A compound (Dpre-03) was obtained in the same manner as in the production of the compound (Dpre-02), except that the compound (K-1) (1.6 g, 16.1 mmol) was changed to the compound (K-2) (2.8 g, 16.1 mmol).

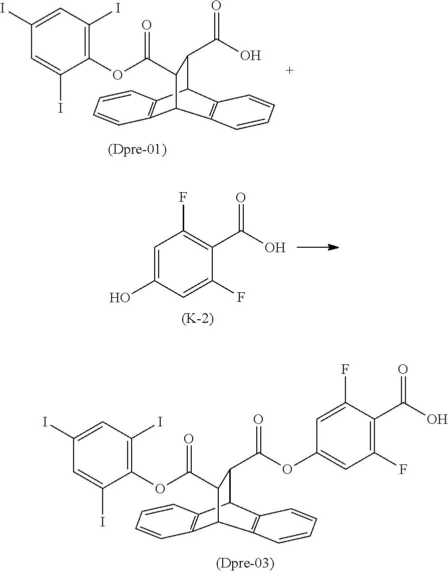

### Production Example 2-4

A compound (Dpre-04) was obtained in the same manner as in the production of the compound (Dpre-02), except that the compound (K-1) (1.6 g, 16.1 mmol) was changed to the compound (K-3) (2.4 g, 16.1 mmol).

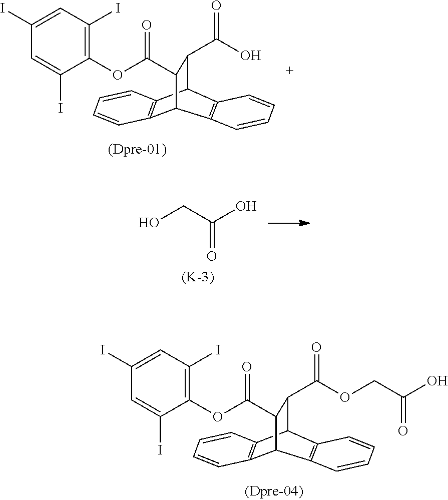

### Production Example 2-5

An intermediate 1 was obtained in the same manner as in the production of the compound (Dpre-02), except that the compound (K-1) (1.6 g, 16.1 mmol) was changed to ethylene glycol (1.0 g, 16.1 mmol).

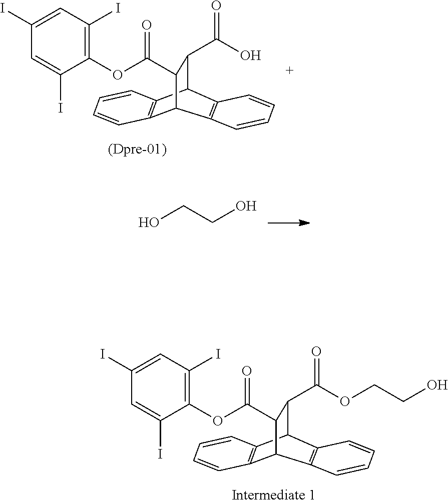

The intermediate 1 (8.5 g, 10.7 mmol), oxalic acid (1.1 g, 11.7 mmol), and dichloromethane (50 g) were charged into a 300 mL three-necked flask and stirred and dissolved at room temperature (25° C.). Next, diisopropylcarbodiimide (1.5 g, 11.7 mmol) and dimethylaminopyridine (0.017 g, 0.2 mmol) were added thereto and reacted at room temperature (25° C.) for 5 hours. The reaction solution was filtered, and the filtrate was concentrated using a rotary evaporator. The concentrate was dissolved in acetonitrile (8 g) and subsequently added dropwise into MTBE (40 g), and the precipitated solid was filtered. The filtrate was dissolved in acetonitrile (8 g) and added dropwise into MTBE (40 g), and the precipitated solid was filtered. After repeating this operation twice, the filtrate was dried under reduced pressure to obtain a compound (Dpre-05).

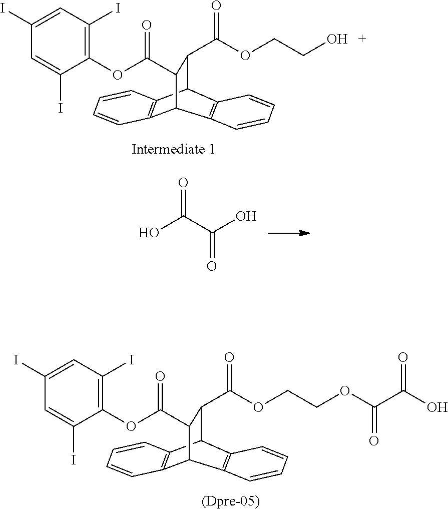

### Production Example 2-6

A compound (Dpre-06) was obtained in the same manner as in the production of (Dpre-01), except that 2,4,6-triiodophenol (25.0 g, 53.0 mmol) was changed to 2,4-diiodophenol (18.3 g, 52.9 mmol).

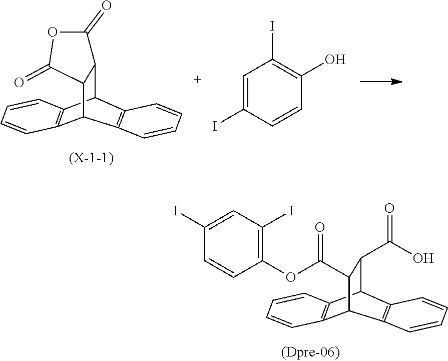

### Production Example 2-7

A compound (Dpre-07) was obtained in the same manner as in the production of (Dpre-0l), except that 2,4,6-triiodophenol (25.0 g, 53.0 mmol) was changed to 4-iodophenol (11.7 g, 53.2 mmol).

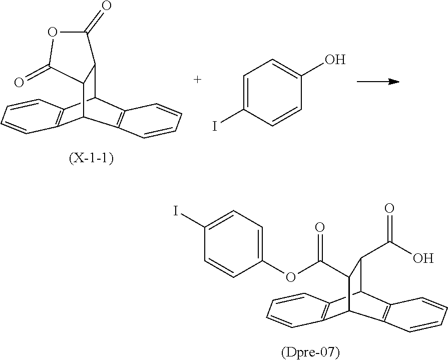

### Production Example 2-8

A compound (Dpre-08) was obtained in the same manner as in the production of (Dpre-01), except that 2,4,6-triiodophenol (25.0 g, 53.0 mmol) was changed to 2-fluoro-4-iodophenol (12.6 g, 52.9 mmol).

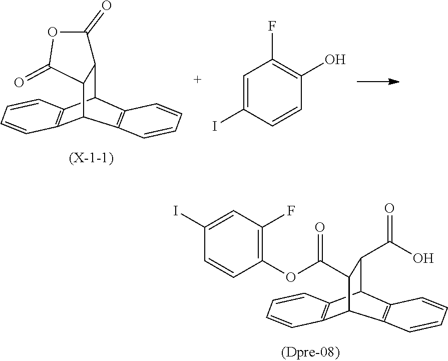

### <Production of Compound (D0)>

### Production Example 3-1

The compound (Dpre-01) (6.0 g, 8.0 mmol) and a compound (S-1-1) (2.86 g, 8.4 mmol) were dissolved in dichloromethane (50 g), and an aqueous solution (14.5 g) of 5% tetramethylammonium hydroxide (TMAH) was added thereto and reacted at room temperature (25° C.) for 30 minutes. After completion of the reaction, the aqueous layer was removed, and the organic layer was washed 5 times with ultrapure water (15.0 g). The organic layer was concentrated and dried using a rotary evaporator to obtain a compound (D0-01).

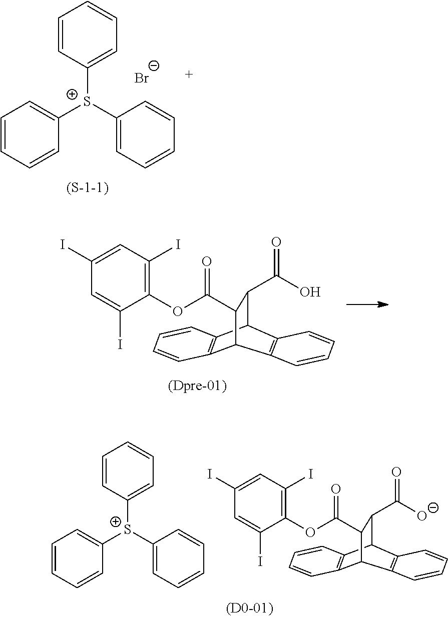 [SMILES: c1ccc([S+](c2ccccc2)c2ccccc2)cc1 ; [Br-] ; O=C(O)[C@H]1C2c3ccccc3C(c3ccccc32)[C@@H]1C(=O)Oc1c(I)cc(I)cc1I ; c1ccc([S+](c2ccccc2)c2ccccc2)cc1 ; O=C([O-])[C@H]1C2c3ccccc3C(c3ccccc32)[C@@H]1C(=O)Oc1c(I)cc(I)cc1I]

### Production Examples 3-2 to 3-11

A compound (D0-02) to a compound (D0-li) described below were obtained in the same manner as in the “production example of the compound (D0-01)” described above, except that the combination of the compound (D0 pre-01) with the salt exchange compound (S-1-1) in the “production example of the compound (D0-0l)” described above was changed to each of the compounds (D0 pre-0l) to (D0 pre-08) described below and each of the salt exchange compounds (S-1-1) to (S-1-4) described below.

The structures of the compound (D0-0l) to the compound (D0-1l) are shown below.

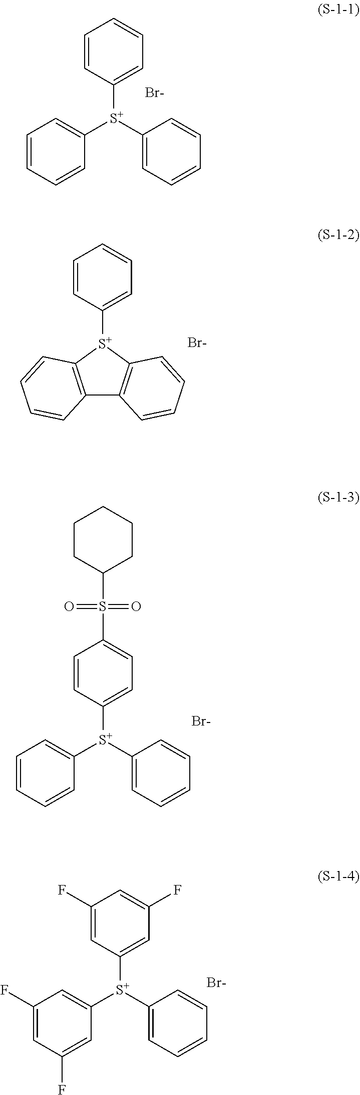

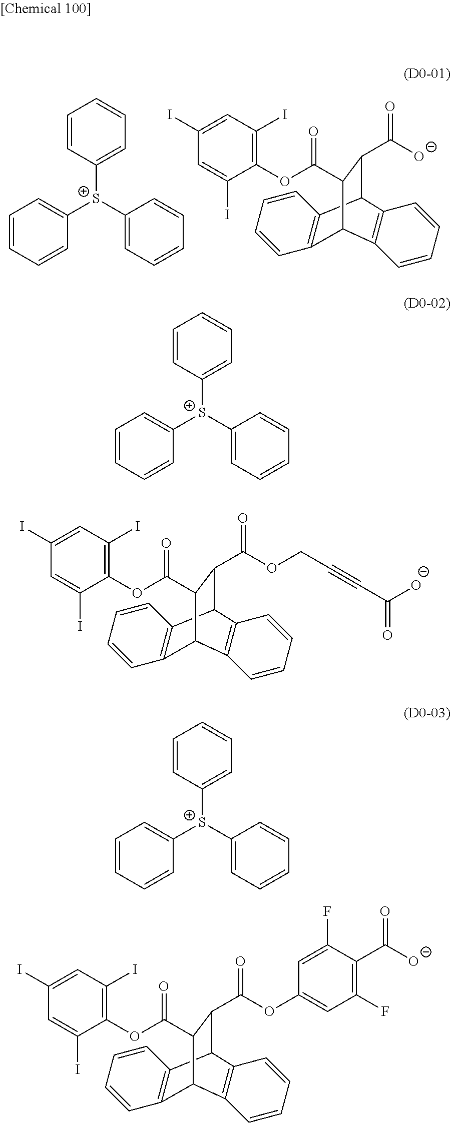 [SMILES: c1ccc([S+](c2ccccc2)c2ccccc2)cc1 ; O=C([O-])C1C2c3ccccc3C(c3ccccc32)C1C(=O)Oc1c(I)cc(I)cc1I ; c1ccc([S+](c2ccccc2)c2ccccc2)cc1 ; O=C([O-])C#CCOC(=O)C1C2c3ccccc3C(c3ccccc32)C1C(=O)Oc1c(I)cc(I)cc1I ; c1ccc([S+](c2ccccc2)c2ccccc2)cc1 ; O=C([O-])c1c(F)cc(OC(=O)C2C3c4ccccc4C(c4ccccc43)C2C(=O)Oc2c(I)cc(I)cc2I)cc1F]

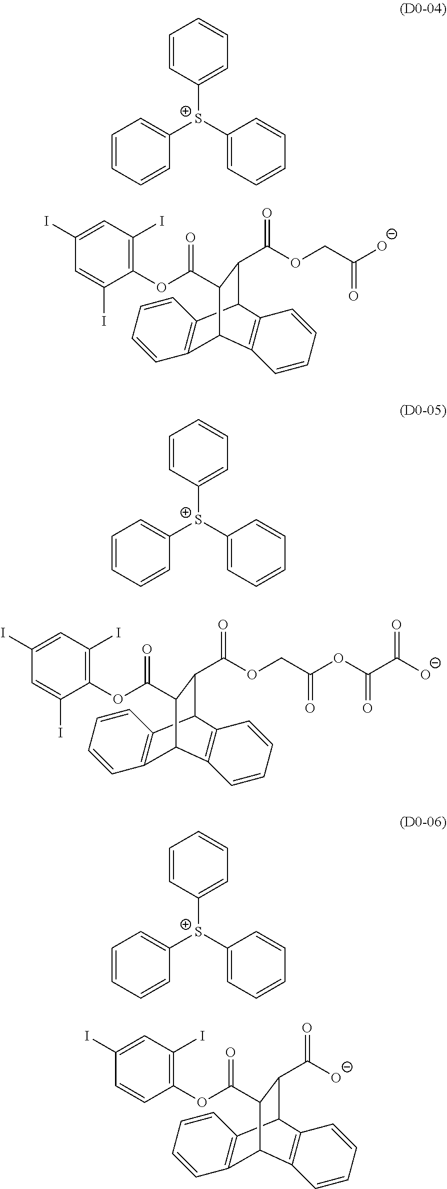 [SMILES: c1ccc([S+](c2ccccc2)c2ccccc2)cc1 ; O=C([O-])COC(=O)C1C2c3ccccc3C(c3ccccc32)C1C(=O)Oc1c(I)cc(I)cc1I ; c1ccc([S+](c2ccccc2)c2ccccc2)cc1 ; O=C(COC(=O)C1C2c3ccccc3C(c3ccccc32)C1C(=O)Oc1c(I)cc(I)cc1I)OC(=O)C(=O)[O-] ; c1ccc([S+](c2ccccc2)c2ccccc2)cc1 ; O=C([O-])C1C2c3ccccc3C(c3ccccc32)C1C(=O)Oc1ccc(I)cc1I]

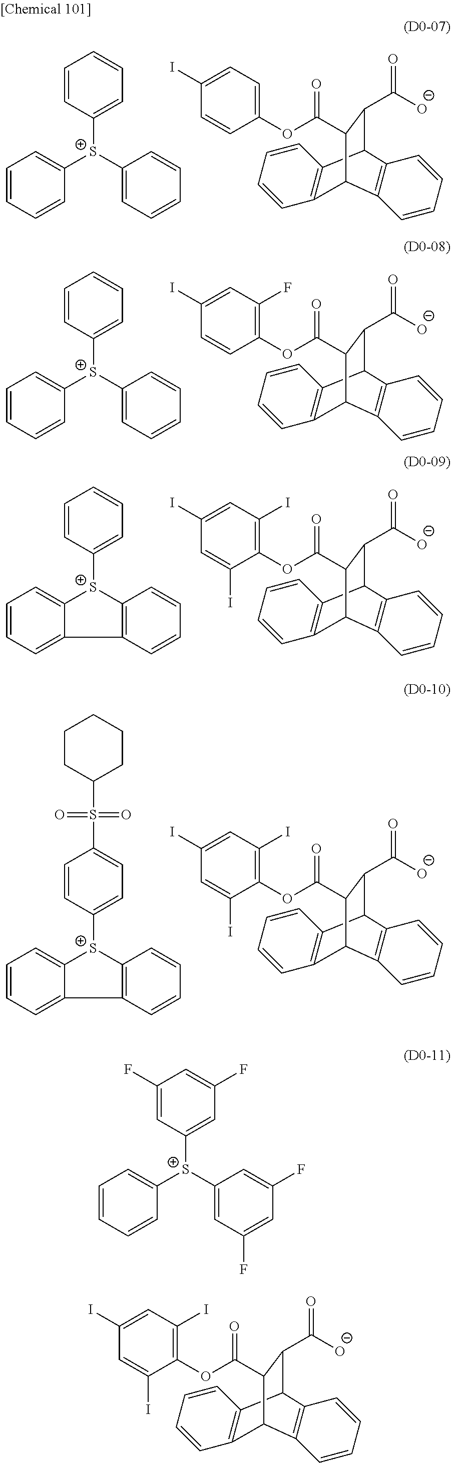

It is noted that the structures of the above-described compounds (D0-01) to (D0-11) were identified from the analysis results of the ¹H-NMR measurement shown below.

Compound (D0-01): A combination of the compound (D0 pre-01) and the salt exchange compound (S-1-1)

¹H-NMR (DMSO, 400 MHz): δ (ppm)=7.99 (d, I-ArH, 2H), 7.90-7.74 (m, ArH, 15H), 7.48-7.43 (m, ArH, 3H), 7.32-7.30 (m, ArH, 4H), 7.17-7.10 (m, ArH, 4H), 4.78 (d, CH, 1H), 4.73 (d, CH, 1H), 3.74-3.72 (m, —OCO—CH—CH—COO, 1H), 2.72-2.70 (m, —OCO—CH—CH—COO, 1H)

Compound (D0-02): A combination of the compound (D0 pre-02) and the salt exchange compound (S-1-1)

¹H-NMR (DMSO, 400 MHz): δ (ppm)=7.99 (d, I-ArH, 2H), 7.90-7.74 (m, ArH, 15H), 7.48-7.43 (m, ArH, 3H), 7.32-7.30 (m, ArH, 1H), 7.17-7.10 (m, ArH, 4H), 5.02 (d, CH, 1H), 4.84 (d, CH, I H), 3.58-3.57 (m, —OCO—CH—CH—COO, 1H), 3.42-3.40 (m, —OCO—CH—CH—COO, 1H), 2.15 (s, —COO—CH₂—, 2H)

Compound (D0-03): A combination of the compound (D0 pre-03) and the salt exchange compound (S-1-1)

¹H-NMR (DMSO, 400 MHz): δ (ppm)=7.99 (d, 1-ArH, 2H), 7.90-7.74 (m, ArH, 15H), 7.48-7.43 (m, ArH, 3H), 7.32-7.30 (m, ArH, 1H), 7.17-7.10 (m, ArH, 4H), 6.73 (s, ArH, 2H), 5.02 (d, CH, 1H), 4.84 (d, CH, 1H), 3.58-3.57 (m, —OCO—CH—CH—COO, 1H), 3.42-3.40 (m, —OCO—CH—CH—COO, 1H)

Compound (D0-04): A combination of the compound (D0 pre-04) and the salt exchange compound (S-1-1)

¹H-NMR (DMSO, 400 MHz): δ (ppm)=7.99 (d, I-ArH, 2H), 7.90-7.74 (m, ArH, 15H), 7.48-7.43 (m, ArH, 3H), 7.32-7.30 (m, ArH, 1H), 7.17-7.10 (m, ArH, 4H), 5.02 (d, CH, 1H), 4.84 (d, CH, 1H), 4.49 (s, —COO—CH₂—, 2H), 3.58-3.57 (m, —OCO—CH—CH—COO, 1H), 3.42-3.40 (m, —OCO—CH—CH—COO, 1H)

Compound (D0-05): A combination of the compound (D0 pre-05) and the salt exchange compound (S-1-1)

¹H-NMR (DMSO, 400 MHz): δ (ppm)=7.99 (d, I-ArH, 2H), 7.90-7.74 (m, ArH, 15H), 7.48-7.43 (m, ArH, 3H), 7.32-7.30 (m, ArH, 1H), 7.17-7.10 (m, ArH, 4H), 5.02 (d, CH, 1H), 4.84 (d, CH, 1H), 3.79-3.75 (m, —COO—CH₂CH₂—COO—, 4H), 3.58-3.57 (m, —OCO—CH—CH—COO, 1H), 3.42-3.40 (m, —OCO—CH—CH—COO, 1H)

Compound (D0-06): A combination of the compound (D0 pre-06) and the salt exchange compound (S-1-1)

¹H-NMR (DMSO, 400 MHz): δ (ppm)=7.98 (d, I-ArH, 1H), 7.90-7.74 (m, ArH, I-ArH, 16H), 7.48-7.43 (m, ArH, 3H), 7.32-7.30 (m, ArH, 1H), 7.17-7.10 (m, ArH, 4H), 6.89 (dd, I-ArH, 1H), 4.78 (d, CH, I H), 4.73 (d, CH, I H), 3.74-3.72 (m, —OCO—CH—CH—COO, 1H), 2.72-2.70 (m, —OCO—CH—CH—COO, 1H)

Compound (D0-07): A combination of the compound (D0 pre-07) and the salt exchange compound (5-1-1)

¹H-NMR (DMSO, 400 MHz): δ (ppm)=7.90-7.74 (m, ArH, I-ArH, 17H), 7.48-7.43 (m, ArH, 3H), 7.32-7.30 (m, ArH, I H), 7.17-7.10 (m, ArH, 4H), 6.87 (dd, I-ArH, 2H), 4.78 (d, CH, I H), 4.73 (d, CH, 1H), 3.74-3.72 (m, —OCO—CH—CH—COO, 1H), 2.72-2.70 (m, —OCO—CH—CH—COO, 1H)

Compound (D0-08): A combination of the compound (D0 pre-08) and the salt exchange compound (S-1-1)

¹H-NMR (DMSO, 400 MHz): δ (ppm)=7.90-7.74 (m, ArH, 15H), 7.48-7.39 (m, ArH, I-ArH, 5H), 7.32-7.30 (m, ArH, 1H), 7.17-7.10 (m, ArH, 4H), 7.00-6.98 (m, 1-ArH, 1H), 4.78 (d, CH, 1H), 4.73 (d, CH, 1H), 3.74-3.72 (m, —OCO—CH—CH—COO, 1H), 2.72-2.70 (m, —OCO—CH—CH—COO, 1H)

Compound (D0-09): A combination of the compound (D0 pre-01) and the salt exchange compound (S-1-2)

¹H-NMR (DMSO, 400 MHz): δ (ppm)=8.50 (d, ArH, 2H), 8.37 (d, ArH, 2H), 7.99 (d, l-ArH, 2H), 7.93 (t, ArH, 2H), 7.75-7.55 (in, Ar, 7H), 7.48-7.43 (m, ArH, 3H), 7.32-7.30 (m, ArH, 1H), 7.17-7.10 (m, ArH, 4H), 4.78 (d, CH, 1H), 4.73 (d, CH, 1H), 3.74-3.72 (m, —OCO—CH—CH—COO, 1H), 2.72-2.70 (m, —OCO—CH—CH—COO, 1H)

Compound (D0-10): A combination of the compound (D0 pre-01) and the salt exchange compound (S-1-3)

¹H-NMR (DMSO, 400 MHz): δ (ppm)=8.22-7.70 (m, ArH, I-ArH, 16H), 7.48-7.43 (m, ArH, 3H), 7.32-7.30 (m, ArH, 1H), 7.17-7.10 (m, ArH, 4H), 4.78 (d, CH, 1H), 4.73 (d, CH, 1H), 3.74-3.72 (m, —OCO—CH—CH—COO, 1H), 2.72-2.70 (m, —OCO—CH—CH—COO, 1H), 2.77 (m, cyclohexyl, 1H), 2.11-1.12 (m, chclohexyl, 1OH)

Compound (D0-111): A combination of the compound (D0 pre-01) and the salt exchange compound (S-1-4)

¹H-NMR (DMSO, 400 MHz): δ (ppm)=7.99-7.77 (m, ArH, I-ArH 13H), 7.48-7.43 (m, ArH, 3H), 7.32-7.30 (m, ArH, 1H), 7.17-7.10 (m, ArH, 4H), 4.78 (d, CH, 1H), 4.73 (d, CH, 1H), 3.74-3.72 (m, —OCO—CH—CH—COO, 1H), 2.72-2.70 (m, —OCO—CH—CH—COO, 1H)

### <Preparation of Resist Composition>

### Examples 1 to 13 and Comparative Examples 1 to 5

Each of the components shown in Tables 1 and 2 was mixed and dissolved to prepare a resist composition of each Example.

TABLE 1

| | Component | Component | Component | Component |
|---|---|---|---|---|
| | (A) | (B) | (D) | (S) |
| Example | (A)-1 | (B)-1 | (D0)-1 | (S)-1 |
| 1 | [100] | [20.0] | [9.9] | [8000] |
| Example | (A)-1 | (B)-1 | (D0)-2 | (S)-1 |
| 2 | [100] | [20.0] | [10.7] | [8000] |
| Example | (A)-1 | (B)-1 | (D0)-3 | (S)-1 |
| 3 | [100] | [20.0] | [11.4] | [8000] |
| Example | (A)-1 | (B)-1 | (D0)-4 | (S)-1 |
| 4 | [100] | [20.0] | [10.4] | [8000] |
| Example | (A)-1 | (B)-1 | (D0)-5 | (S)-1 |
| 5 | [100] | [20.0] | [11.0] | [8000] |
| Example | (A)-1 | (B)-1 | (D0)-6 | (S)-1 |
| 6 | [100] | [20.0] | [8.6] | [8000] |
| Example | (A)-1 | (B)-1 | (D0)-7 | (S)-1 |
| 7 | [100] | [20.0] | [7.4] | [8000] |
| Example | (A)-1 | (B)-1 | (D0)-8 | (S)-1 |
| 8 | [100] | [20.0] | [7.6] | [8000] |
| Example | (A)-1 | (B)-1 | (D0)-9 | (S)-1 |
| 9 | [100] | [20.0] | [9.8] | [8000] |
| Example | (A)-1 | (B)-1 | (D0)-10 | (S)-1 |
| 10 | [100] | [20.0] | [11.3] | [8000] |
| Example | (A)-1 | (B)-1 | (D0)-11 | (S)-1 |
| 11 | [100] | [20.0] | [10.6] | [8000] |
| Example | (A)-1 | (B)-2 | (D0)-1 | (S)-1 |
| 12 | [100] | [19.9] | [9.9] | [8000] |
| Example | (A)-2 | (B)-1 | (D0)-1 | (S)-1 |
| 13 | [100] | [20.0] | [9.9] | [8000] |

TABLE 2

| | Component | Component | Component | Component |
|---|---|---|---|---|
| | (A) | (B) | (D) | (S) |
| Comparative | (A)-1 | (B)-1 | (D1)-1 | (S)-1 |
| Example | [100] | [20.0] | [5.0] | [8000] |
| 1 | | | | |
| Comparative | (A)-1 | (B)-1 | (D1)-2 | (S)-1 |
| Example | [100] | [20.0] | [5.4] | [8000] |
| 2 | | | | |
| Comparative | (A)-1 | (B)-1 | (D1)-3 | (S)-1 |
| Example | [100] | [20.0] | [8.7] | [8000] |
| 3 | | | | |
| Comparative | (A)-1 | (B)-1 | (D1)-4 | (S)-1 |
| Example | [100] | [20.0] | [8.8] | [8000] |
| 4 | | | | |
| Comparative | (A)-1 | (B)-1 | (D1)-5 | (S)-1 |
| Example | [100] | [20.0] | [8.6] | [8000] |
| 5 | | | | |

The abbreviations in Tables 1 and 2 have the following meanings. The numerical values in the brackets are blending amounts (parts by mass).
(A)-1: The polymeric compound represented by Chemical Formula (A1)-1 described below. As found through a GPC measurement to determine the weight average molecular weight (Mw) in terms of the standard polystyrene equivalent value, this polymeric compound (A1)-1 had a weight average molecular weight of 7,100 and a molecular weight polydispersity (Mw/Mn) of 1.69. The copolymerization compositional ratio (the ratio (molar ratio) among constitutional units in the structural formula) determined by ¹³C-NMR was 1/m=50/50.
(A)-2: The polymeric compound represented by Chemical Formula (A1)-2 described below. As found through a GPC measurement to determine the weight average molecular weight (Mw) in terms of the standard polystyrene equivalent value, this polymeric compound (A1)-2 had a weight average molecular weight of 7,000 and a molecular weight polydispersity (Mw/Mn) of 1.72. The copolymerization compositional ratio (the ratio (molar ratio) among constitutional units in the structural formula) determined by ¹³C-NMR was/in =50/50.

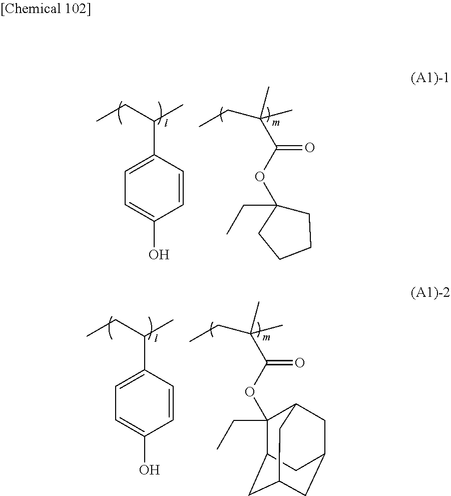

(B)-1: An acid generator consisting of the compound (B1-1) described below.
(B)-2: An acid generator consisting of the compound (B1-2) described below.

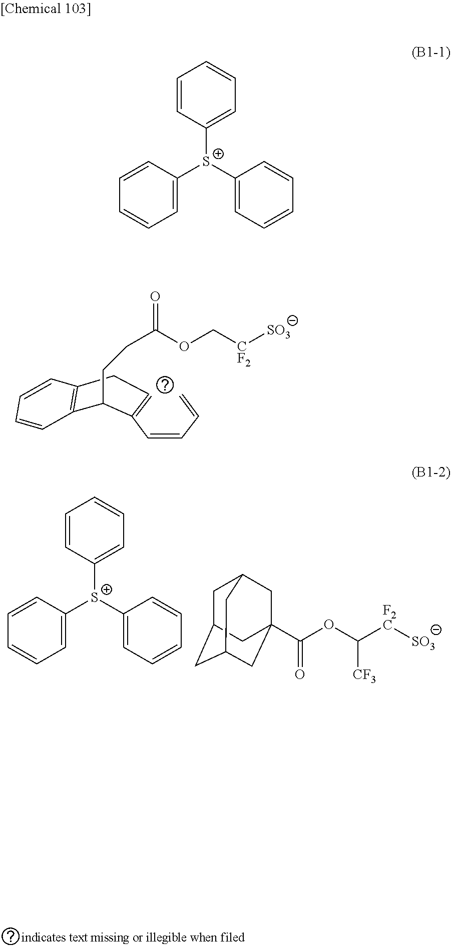

(D0)-1 to (D0)-11: Acid diffusion control agents consisting of each of the compounds (D-01) to (D0-11) described above.
(D1)-11 to (D1)-5: Acid diffusion control agent components consisting of each of the compounds (D1-1) to (D1-5) described below.
(S)-1: A mixed solvent of propylene glycol monomethyl ether acetate/propylene glycol monomethyl ether=60/40 (in terms of mass ratio)

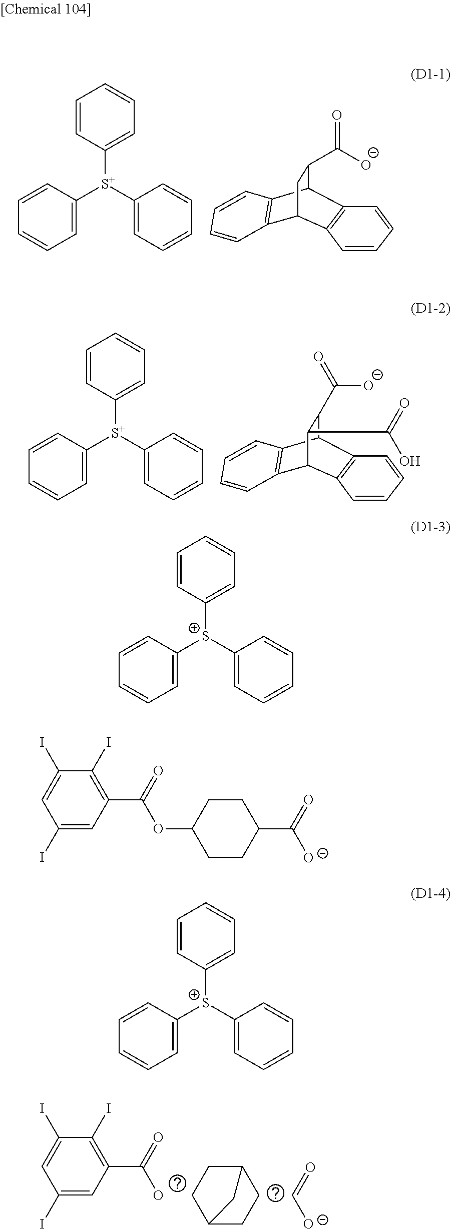

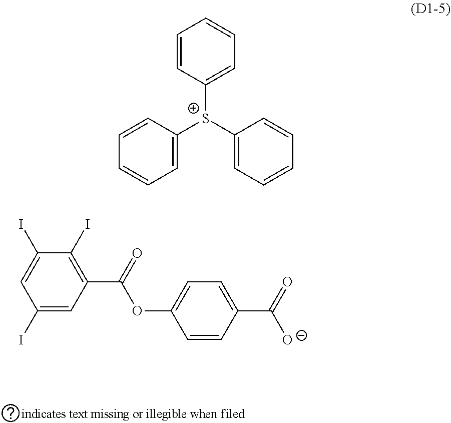 [SMILES: c1ccc([S+](c2ccccc2)c2ccccc2)cc1 ; O=C([O-])c1ccc(OC(=O)c2cc(I)cc(I)c2I)cc1]

### <Resist Pattern Formation>

The resist composition of each Example was applied onto an 8-inch silicon substrate which had been subjected to a hexamethyldisilazane (HMDS) treatment using a spinner, and the coated wafer was subjected to a pre-apply bake (PAB) treatment on a hot plate at a temperature of 110° C. for 60 seconds so that the coated wafer was dried to form a resist film having a film thickness of 50 nm.

Next, the resist film was subjected to drawing (exposure) to obtain a contact hole pattern (hereinafter, referred to as a “CH pattern”) in which holes having a diameter of 32 nm were arranged at equal intervals (pitch: 64 nm) by using an electron beam lithography apparatus JEOL-JBX-9300FS (manufactured by JEOL Ltd.) at an acceleration voltage of 100 kV. Thereafter, a post-exposure bake (PEB) treatment was carried out on the resist film at 110° C. for 60 seconds.

Subsequently, alkali development was carried out at 23° C. for 60 seconds using a 2.38% by mass tetramethylammonium hydroxide (TMAH) aqueous solution “NMD-3” (product name, manufactured by TOKYO OHKA KOGYO CO., LTD.).

Thereafter, water rinsing was carried out with pure water for 15 seconds.

As a result of the above, a CH pattern in which holes having a diameter of 32 nm were arranged at equal intervals (pitch: 64 nm) was formed.

### [Evaluation of Optimum Exposure Amount (Eop)]

According to <Resist pattern formation>described above, an optimum exposure amount Eop (μC/cm²) at which a CH pattern having the target size was formed was determined. The results are shown in Table 3 as “Eop (pC/cm²)”.

### [Evaluation of Critical Dimension Uniformity (CDU) of Pattern Size]

The CH pattern formed according to “Resist pattern formation” described above was observed from the upper side of the CH pattern, and the hole diameter (nm) of each of the holes was measured with a length-measuring scanning electron microscope (SEM, acceleration voltage: 500 V, product name: CG5000, manufactured by Hitachi High-Tech Corporation). Then, the triple value (3σ) of the standard deviation (σ) calculated from the measurement result was determined. The obtained results are shown in Table 3 as “CDU (nm)”.

The lower the value of 36 determined as described above is, the higher the critical dimension (CD) uniformity of the plurality of holes formed in the resist film is.

TABLE 3

| | PAB | PEB | Eop | CDU |
|---|---|---|---|---|
| | (° C.) | (° C.) | [μC/cm²] | [nm] |
| Example 1 | 110 | 110 | 88 | 4.1 |
| Example 2 | 110 | 110 | 88 | 4.0 |
| Example 3 | 110 | 110 | 87 | 3.9 |
| Example 4 | 110 | 110 | 88 | 4.0 |
| Example 5 | 110 | 110 | 87 | 3.9 |
| Example 6 | 110 | 110 | 90 | 4.1 |
| Example 7 | 110 | 110 | 93 | 4.4 |
| Example 8 | 110 | 110 | 93 | 4.4 |
| Example 9 | 110 | 110 | 87 | 3.8 |
| Example 10 | 110 | 110 | 87 | 3.8 |
| Example 11 | 110 | 110 | 84 | 3.6 |
| Example 12 | 110 | 110 | 88 | 4.1 |
| Example 13 | 110 | 110 | 88 | 4.0 |
| Comparative | 110 | 110 | 115 | 5.0 |
| Example 1 | | | | |
| Comparative | 110 | 110 | 116 | 5.2 |
| Example 2 | | | | |
| Comparative | 110 | 110 | 88 | 5.5 |
| Example 3 | | | | |
| Comparative | 110 | 110 | 87 | 5.4 |
| Example 4 | | | | |
| Comparative | 110 | 110 | 87 | 5.6 |
| Example 5 | | | | |

As shown in Table 3, it could be confirmed that the resist compositions of Examples have high sensitivity in the resist pattern formation and have good CDU as compared with the resist compositions of Comparative Examples.

Although the resist compositions of Examples 1, 6, and 7 have the same component (D0) in the main skeleton, they are different from each other in the number of iodine atoms in the anion moiety of the component (D0). The compound (D0-01) contained in the resist composition of Example 1 has three iodine atoms, the compound (D0-06) contained in the resist composition of Example 6 has two iodine atoms, and the compound (D0-07) contained in the resist composition of Example 7 has one iodine atom.

Since the resist composition of Example 1 had good sensitivity and good CDU as compared with the resist compositions of Examples 6 and 7, it was confirmed that sensitivity and CDU are improved in a case where the number of iodine atoms contained in the anion moiety of the component (D0) is increased from 1 to 3.

In addition, it was found from the comparison between the resist composition of Example 7 and the resist composition of Example 8 that there is no difference in sensitivity or CDU depending on the presence or absence of a fluorine atom in the anion moiety of the component (D0).

The resist compositions of Examples 1 and 9 to 11 each contain the component (D0) in which the anion moieties are the same but the cation moieties are different from each other.

Since the resist compositions of Examples 9 to 11 had good sensitivity and good CDU as compared with the resist compositions of Example 1, it was found that sensitivity and CDU are improved in a case where the decomposability of the cation moiety of the component (D0) is improved.

The resist composition of Comparative Example 3 contains an acid diffusion control agent consisting of the compound (D1-3) having a monocyclic alicyclic hydrocarbon group. The resist composition of Comparative Example 4 contains an acid diffusion control agent consisting of the compound (D1-4) having a bridged ring-based polycyclic alicyclic hydrocarbon group. The resist composition of Comparative Example 5 contains an acid diffusion control agent consisting of the compound (D1-5) having a monocyclic aromatic hydrocarbon group. Since these resist compositions did not contain an acid diffusion control agent having a condensed cyclic group in which an aromatic ring and an alicyclic ring were condensed as in the resist compositions of Examples, CDU was poor as compared with the resist compositions of Examples.

While preferred examples according to the present invention have been described above, the present invention is not limited to these examples. Additions, omissions, substitutions, and other modifications can be made without departing from the gist of the present invention. Accordingly, the present invention is not to be considered as being limited by the foregoing description and is only limited by the scope of the appended claims.

What is claimed is:

1. A resist composition that generates an acid upon exposure and exhibits changed solubility in a developing solution under an action of the acid, the resist composition comprising:
a base material component (A) that exhibits changed solubility in a developing solution under the action of the acid; and
an acid diffusion control agent component (D) that controls diffusion of the acid generated upon exposure,
wherein the acid diffusion control agent component (D) contains a compound (D0) represented by General Formula (d0), 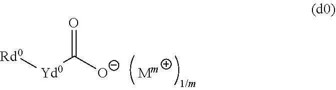
wherein Rd⁰ represents a condensed cyclic group in which an aromatic ring and an alicyclic ring are condensed, the alicyclic ring in the condensed cyclic group has a substituent, where at least one of the substituents includes a hydrocarbon group having a bromine atom or a hydrocarbon group having an iodine atom, Yd⁰ represents a divalent linking group or a single bond, provided that Yd⁰ is bonded to the alicyclic ring in the condensed cyclic group, M^{m+} represents an m-valent organic cation, and m represents an integer of 1 or morel.

2. The resist composition according to claim 1, wherein the aromatic ring in Rd⁰ is a benzene ring.

3. The resist composition according to claim 1,
wherein the acid diffusion control agent component (D) contains a compound represented by General Formula (d0-1), 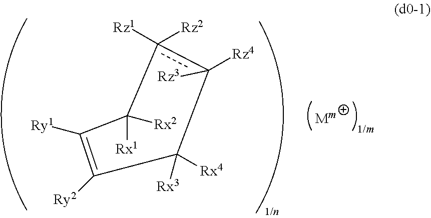
wherein Rx¹ to Rx⁴ each independently represents a hydrocarbon group which may have a substituent or a hydrogen atom, or two or more of Rx¹ to Rx⁴ may be bonded to each other to form a ring structure, and Ry¹ and Ry² each independently represents a hydrocarbon group which may have a substituent or a hydrogen atom, or may be bonded to each other to form a ring structure, [symbol]
the above double line of a dotted line and a straight line represents a double bond or a single bond, where valence permits, Rz¹ to Rz⁴ each independently represents a hydrocarbon group which may have a substituent or a hydrogen atom, or two or more of Rz¹ to Rz⁴ may be bonded to each other to form a ring structure, provided that at least two or more of Rx¹ to Rx⁴, at least Ry¹ and Ry², or at least two or more of Rz¹ to Rz⁴ are bonded to each other to form an aromatic ring, and at least one among Rx¹ to Rx⁴, Ry¹ and Ry², and Rz¹ to Rz⁴ has an anion group represented by General Formula (d0-r-an1), wherein an entire anion moiety is an n-valent anion, and at least one among Rx¹ to Rx⁴, Ry¹ and Ry², and Rz¹ to Rz⁴ includes a hydrocarbon group having a bromine atom or a hydrocarbon group having an iodine atom, n represents an integer of 1 or more, and M^{m+} represents an m-valent organic cation, wherein m represents an integer of 1 or more], 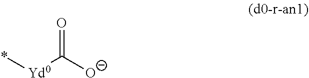
wherein Yd⁰ represents a divalent linking group or a single bond, and * represents a bonding site.

4. The resist composition according to claim 1, wherein a hydrocarbon group in the hydrocarbon group having a bromine atom and the hydrocarbon group having an iodine atom is an aromatic hydrocarbon group.

5. The resist composition according to claim 1, further comprising an acid generator component (B) that generates the acid upon exposure.

6. A resist pattern forming method, comprising:
forming a resist film on a support using the resist composition according to claim 1;
exposing the resist film; and
developing the exposed resist film to form a resist pattern.

7. A compound represented by General Formula (d0), 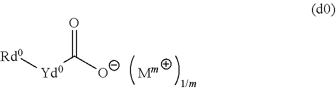
wherein Rd⁰ represents a condensed cyclic group in which an aromatic ring and an alicyclic ring are condensed, the alicyclic ring in the condensed cyclic group has a substituent, at least one of the substituents includes a hydrocarbon group having an iodine atom, Yd⁰ represents a divalent linking group or a single bond, provided that Yd⁰ is bonded to the alicyclic ring in the condensed cyclic group, M^{m+} represents an m-valent organic cation, and m represents an integer of 1 or more.

8. An acid diffusion control agent comprising the compound according to claim 7.
